(19) United States
(12) Patent Application Publication
(10) Pub. No.: US 2020/0262869 A1
(43) Pub. Date: Aug. 20, 2020

(54) PEPTIDE ANTIBIOTIC COMPLEXES AND METHODS OF USE THEREOF

(71) Applicant: GENENTECH, INC., SOUTH SAN FRANCISCO, CA (US)

(72) Inventors: PETER ANDREW SMITH, BRISBANE, CA (US); JEREMY MARK MURRAY, OAKLAND, CA (US); MICHAEL FRIEDRICH THOMAS KOEHLER, PALO ALTO, CA (US); CHRISTOPHER E. HEISE, BELMONT, CA (US)

(21) Appl. No.: 16/829,963

(22) Filed: Mar. 25, 2020

Related U.S. Application Data
(63) Continuation of application No. PCT/US2018/052791, filed on Sep. 26, 2018.
(60) Provisional application No. 62/566,125, filed on Sep. 29, 2017.

(51) Int. Cl. C07K 7/06; C12N 9/52; C12N 9/96; A61P 31/04; A61P 31/06; A61K 38/08; A61K 45/06

(52) U.S. Cl. CPC C07K 7/06; C12N 9/52; C12Y 304/21089; A61K 45/06; A61P 31/04; A61P 31/06; A61K 38/08; C12N 9/96

(57) ABSTRACT

Provided herein are compounds that form covalent bonds with peptidases, wherein the compounds in some embodiments have broad spectrum bioactivity. In various embodiments, the compounds act by inhibition of bacterial type 1 signal peptidase (SPase), an essential protein in bacteria. Pharmaceutical compositions and methods for treatment using the compounds described herein are also provided.

### CROSS-REFERENCE

This application claims the benefit of U.S. Application Ser. No. 62/566,125, filed Sep. 29, 2017, which is incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 24, 2018, is named P34460-WO_Sequence-Listing.TXT and is 2,461 bytes in size.

### BACKGROUND

Antibiotic resistance is a serious and growing phenomenon in contemporary medicine and has emerged as a major public health concern of the 21st century. Therefore, novel classes of broad-spectrum antibiotics, especially those that target novel mechanisms of action, are needed to treat multidrug-resistant pathogens.

### BRIEF SUMMARY OF THE INVENTION

In an aspect provided herein, is an inhibited peptidase. The inhibited peptidase is a signal peptidase (SPase) inhibitor having a bond to an amino acid residue of a bacterial type I SPase, a bacterial type I SPase homolog, or a bacterial type I SPase lysine homolog.

In an aspect provided herein, is an inhibited peptide. The inhibited peptide includes a serine-lysine catalytic dyad or a serine-serine lysine catalytic triad and a peptide inhibitor having a bond to an amino group of the lysine.

In another aspect is provided method of inhibiting a bacterial peptidase, comprising contacting a bacterial cell with a compound as described herein, including embodiments, or the structural Formulae (IV), (IV-C), (V), (V-A), (V-B), (V-C), or (V-D) or a pharmaceutically acceptable salt thereof.

In another aspect is provided a method of inhibiting signal protein secretion of a bacterial cell, comprising contacting the cell with a compound as described herein, including embodiments, or the structural Formulae (IV), (IV-C), (V), (V-A), (V-B), (V-C), or (V-D) or a pharmaceutically acceptable salt thereof.

In another aspect is provided a method of treating a bacterial infection, comprising administering to a subject in need thereof a therapeutically effective amount of a compound as described herein, including embodiments, or the structural Formulae (IV), (IV-C), (V), (V-A), (V-B), (V-C), or (V-D) or a pharmaceutically acceptable salt thereof.

In another aspect is provided a method of inhibiting a peptide, comprising contacting a peptide comprising a serine-lysine catalytic dyad or a serine-serine lysine catalytic triad with a compound as described herein, including embodiments, or the structural Formulae (IV), (IV-C), (V), (V-A), (V-B), (V-C), or (V-D) or a pharmaceutically acceptable salt thereof, wherein the compound forms a bond to an amino group of the lysine in the dyad or triad.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1. Chemical structures of arylomycin A-C₁₆ and G0775. Shaded areas indicate the N-terminal lipopeptide “tail,” location of phenolic oxygen modifications to the arylomycin core macrocycle, and the site for appendage of the nitrile electrophile that covalently engages the LepB catalytic lysine

FIGS. 2A-2B. G0775 resistance. Frequencies of resistance (FOR) of E. coli ATCC 25922, K. pneumoniae 43816, P. aeruginosa PAO1, and A. baumannii ATCC 17978 to G0775 at 4, 8 and 16-fold each strains respective MIC. Limit of detection (10⁻¹⁰) is demarked by dotted line. Data shown are an average of at least three independent experiments. Error bars represent the standard deviation (FIG. 2A). LepB target mutations mapped onto the G0775-LepB crystal structure. Point mutants were spontaneously generated in E. coli ATCC 25922 by overnight plating on G0775 at 4×MIC. G0775 is shown in stick representation (FIG. 2B).

FIGS. 3A-3C. G0775 binds LepB protease domain to form an irreversible covalent bond with catalytic lysine 146. Crystal structure at 2.8 Å resolution of the protease domain of LepB with G0775 is represented as sticks covalently bound to lysine 146 (FIG. 3A). LCMS detection of LDYIKR LepB peptide fragment (SEQ ID NO: 5) after tryptic digest following incubation of LepB with G0775 (FIG. 3B). LepB kinetic enzyme assays in the presence of the indicated concentration of G0775. K_{inact} (0.0007±0.0002 s⁻¹) and K_{I} (0.44±0.15 M⁻¹ s⁻¹) were measured from three independent experiments and the data points shown are averages of four replicates from a single experiment (FIG. 3C.)

FIGS. 4A-4C. In vivo efficacy of G0775. Thigh infections initiated in neutropenic mice with the indicated Gram-negative bacterial species were treated with G0775 or vehicle, and bacterial burden was quantified 20 hours after infection. G0775 was delivered subcutaneously twice during the infection period (BID) at 2 and 11 hours post infection at the indicated dose (FIG. 4A). Dose-dependent antibacterial activity of G0775 against a bacterial infection established by the MDR K. pneumoniae clinical isolate CDC 0106 in the lungs of neutropenic mice. G0775 was delivered subcutaneously twice during the 20 hour infection period (BID) at 2 and 11 hours post infection initiation (FIG. 4B). Kaplan-Meier survival analysis of mice peritoneally infected with K. pneumoniae ATCC 43816. G0775 was delivered subcutaneously twice on day zero at 2 and 11 hours post infection initiation and animal viability (n=8) was monitored out to 84 hours (FIG. 4C). Broken red lines in FIGS. 4A and 4B represent the limit of bacterial CFU determination.

FIG. 5. Time-kill of E. coli ATCC 25922 when measured at 0.25, 1, 4 and 16-fold the measured G0775 MIC (0.125 μg/ml). Colony forming units (CFU) per mL data shown is an average of at least three independent experiments with error bars representing the SD.

FIG. 6. Overlay of LepB-G0775 and LepB-Arylomycin from PDB 1T7D.

FIG. 7. Proposed mechanism of covalent amidine bond formation between G0775 nitrile and lysine 146.

FIGS. 8A-8B. Electron density and space groups of the G0775-LepB co-structure. The LepB-G0775 complex crystallized in spacegroup I4 with two molecules in the asymmetric unit (FIG. 8A). The 2fofc electron density for each inhibitor molecule in the asymmetric unit (FIG. 8B).

FIGS. 9A-9C. G0775 MIC values measured against a challenge panel of 49 multi-drug resistant clinical isolates obtained from the Centers for Disease Control and Prevention (CDC).

FIGS. 10A-10B. Genetic determinants of resistance identified in K. pneumoniae CDC 0106 using whole genome sequencing. *MICs determined by the CDC. ^{†}Differs from the MIC determined by Genentech and reported in Table 2.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

As used herein, “individual” (as in the subject of the treatment) means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “disease” or “disorder” or “malcondition” are used interchangeably, and are used to refer to diseases or conditions wherein a bacterial SPase plays a role in the biochemical mechanisms involved in the disease or malcondition such that a therapeutically beneficial effect can be achieved by acting on the enzyme. “Acting on” SPase can include binding to SPase and/or inhibiting the bioactivity of an SPase.

The expression “effective amount”, when used to describe therapy to an individual suffering from a disorder, refers to the amount of a compound described herein that is effective to inhibit or otherwise act on SPase in the individual's tissues wherein SPase involved in the disorder is active, wherein such inhibition or other action occurs to an extent sufficient to produce a beneficial therapeutic effect.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

When a substituent is specified to be an atom or atoms of specified identity, “or a bond,” a configuration is referred to when the substituent is “a bond” that the groups that are immediately adjacent to the specified substituent are directly connected to each other in a chemically feasible bonding configuration.

All chiral, diastereomeric, racemic forms of a structure are intended, unless a particular stereochemistry or isomeric form is specifically indicated. Compounds described herein can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

The inclusion of an isotopic form of one or more atoms in a molecule that is different from the naturally occurring isotopic distribution of the atom in nature is referred to as an “isotopically labeled form” of the molecule. All isotopic forms of atoms are included as options in the composition of any molecule, unless a specific isotopic form of an atom is indicated. For example, any hydrogen atom or set thereof in a molecule can be any of the isotopic forms of hydrogen, i.e., protium (¹H), deuterium (²H), or tritium (³H) in any combination. Similarly, any carbon atom or set thereof in a molecule can be any of the isotopic form of carbons, such as ¹¹C, ¹²C ¹³C, or ¹⁴C, or any nitrogen atom or set thereof in a molecule can be any of the isotopic forms of nitrogen, such as ¹³N, ¹⁴N, or ¹⁵N. A molecule can include any combination of isotopic forms in the component atoms making up the molecule, the isotopic form of every atom forming the molecule being independently selected. In a multi-molecular sample of a compound, not every individual molecule necessarily has the same isotopic composition. For example, a sample of a compound can include molecules containing various different isotopic compositions, such as in a tritium or ¹⁴C radiolabeled sample where only some fraction of the set of molecules making up the macroscopic sample contains a radioactive atom. It is also understood that many elements that are not artificially isotopically enriched themselves are mixtures of naturally occurring isotopic forms, such as ¹⁴N and ¹⁵N, ³²S and ³⁴S, and so forth. A molecule as recited herein is defined as including isotopic forms of all its constituent elements at each position in the molecule. As is well known in the art, isotopically labeled compounds can be prepared by the usual methods of chemical synthesis, except substituting an isotopically labeled precursor molecule. The isotopes, radiolabeled or stable, can be obtained by any method known in the art, such as generation by neutron absorption of a precursor nuclide in a nuclear reactor, by cyclotron reactions, or by isotopic separation such as by mass spectrometry. The isotopic forms are incorporated into precursors as required for use in any particular synthetic route. For example, ¹⁴C and ³H can be prepared using neutrons generated in a nuclear reactor. Following nuclear transformation, ¹⁴C and ³H are incorporated into precursor molecules, followed by further elaboration as needed.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The term “hydroxyl protecting group” or “O-protected” as used herein refers to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M., John Wiley & Sons, New York, N.Y., (3rd Edition, 1999). Hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

In general, “substituted” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, NO, NO₂, ONO₂, azido, CF₃, OCF₃, R′, O (oxo), S (thiono), C(O), S(O), methylenedioxy, ethylenedioxy, N(R′)₂, SR′, SOR′, SOzR′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” group.

Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)n wherein n is 1, 2, 3, or more, and each R′ is independently selected.

C(O) and S(O)₂ groups can be bound to one or two heteroatoms, such as nitrogen, rather than to a carbon atom. For example, when a C(O) group is bound to one carbon and one nitrogen atom, the resulting group is called an “amide” or “carboxamide.” When a C(O) group is bound to two nitrogen atoms, the functional group is termed a urea. When a S(O)₂ group is bound to one carbon and one nitrogen atom, the resulting unit is termed a “sulfonamide.” When a S(O)₂ group is bound to two nitrogen atoms, the resulting unit is termed a “sulfamate.”

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

As to any of the groups described herein, which contain one or more substituents, it is understood, of course, that such groups do not contain any substitution or substitution patterns which are sterically impractical and/or synthetically non-feasible. In addition, the compounds of this disclosed subject matter include all stereochemical isomers arising from the substitution of these compounds.

Selected substituents within the compounds described herein are present to a recursive degree. In this context, “recursive substituent” means that a substituent may recite another instance of itself or of another substituent that itself recites the first substituent. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the disclosed subject matter. One of ordinary skill in the art of medicinal and organic chemistry understands the versatility of such substituents. To the degree that recursive substituents are present in a claim of the disclosed subject matter, the total number should be determined as set forth above.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structures are substituted therewith.

The term “thioalkoxy” refers to an alkyl group previously defined attached to the parent molecular moiety through a sulfur atom.

The term “glycosyloxyoxy” refers to a glycoside attached to the parent molecular moiety through an oxygen atom.

The term “alkoxycarbonyl” represents as ester group; i.e. an alkoxy group, attached to the parent molecular moiety through a carbonyl group such as methoxycarbonyl, ethoxycarbonyl, and the like.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed above, for example, amino, hydroxy, cyano, carboxy, nitro, thio, alkoxy, and halogen groups.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “carbonyl” means C═O.

The terms “carboxy” and “hydroxycarbonyl” mean COOH.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain.

Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7.

Cycloalkyl groups further include polycyclic cycloalkyl groups such as, but not limited to, norbornyl, adamantyl, bomyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, —CH₂—CH═CH—CH₂—SH, and and —CH═CH—O—CH₂CH₂—O—CH₃.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. “Cycloalkyl” is also meant to refer to bicyclic and polycyclic hydrocarbon rings such as, for example, bicyclo[2.2.1]heptane, bicyclo[2.2.2]octane, etc.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group as defined above is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

Heterocyclyl groups or the term “heterocyclyl” includes aromatic and non-aromatic ring compounds containing 3 or more ring members, of which, one or more is a heteroatom such as, but not limited to, N, O, and S. Thus a heterocyclyl can be a heterocycloalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

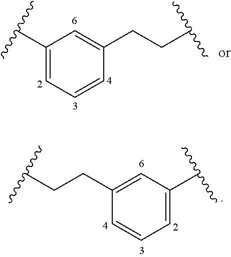 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

A “haloalkoxy” group includes mono-halo alkoxy groups, poly-halo alkoxy groups wherein all halo atoms can be the same or different, and per-halo alkoxy groups, wherein all hydrogen atoms are replaced by halogen atoms, such as fluoro. Examples of haloalkoxy include trifluoromethoxy, 1,1-dichloroethoxy, 1,2-dichloroethoxy, 1,3-dibromo-3,3-difluoropropoxy, perfluorobutoxy, and the like.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂—.

The terms “aryloxy” and “arylalkoxy” refer to, respectively, an aryl group bonded to an oxygen atom and an aralkyl group bonded to the oxygen atom at the alkyl moiety. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

An “amino” group is a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary or quaternary amino group. An “alkylamino” group includes a monoalkylamino, dialkylamino, and trialkylamino group.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to primary carboxamide groups (—C(O)NH₂) and formamide groups (—NHC(O)H). A “carboxamido” or “aminocarbonyl” group is a group of the formula C(O)NR₂, wherein R can be H, alkyl, aryl, etc.

The term “azido” refers to an N₃ group. An “azide” can be an organic azide or can be a salt of the azide (N₃⁻) anion. The term “nitro” refers to an NO₂ group bonded to an organic moiety. The term “nitroso” refers to an NO group bonded to an organic moiety. The term nitrate refers to an ONO₂ group bonded to an organic moiety or to a salt of the nitrate (NO₃⁻) anion.

The term “urethane” (“carbamoyl” or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

The term “ring derived from a sugar” refers to a compound that forms a ring by removing the hydrogen atoms from two hydroxyl groups of any sugar.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds described herein may take the form of salts. The term “salts” embraces addition salts of free acids or free bases which are compounds described herein. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present disclosure, such as for example utility in process of synthesis, purification or formulation of compounds of the present disclosure.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the present disclosure include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of compounds of Formulae (IV), (IV-C), (V), (V-A), (V-B), (V-C), or (V-D), including embodiments, for example in their purification by recrystallization. All of these salts may be prepared by conventional means from the corresponding compound according to Formulae (IV), (IV-C), (V), (V-A), (V-B), (V-C), or (V-D), including embodiments, by reacting, for example, the appropriate acid or base with the compound according to Formulae (IV), (IV-C), (V), (V-A), (V-B), (V-C), or (V-D), including embodiments. The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to a patient where the substance is converted in vivo by the action of biochemicals within the patients body, such as enzymes, to the active pharmaceutical ingredient. Examples of prodrugs include esters of carboxylic acid groups, which can be hydrolyzed by endogenous esterases as are found in the bloodstream of humans and other mammals. Further examples examples of prodrugs include boronate esters which can be hydrolyzed under physiological conditions to afford the corresponding boronic acid. Conventional procedures for the selection and preparation of suitable prodrug derivatives are described, for example, in “Design of Prodrugs”, ed. H. Bundgaard, Elsevier, 1985.

In addition, where features or aspects of the present disclosure are described in terms of Markush groups, those skilled in the art will recognize that the presently described compounds is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the present disclosure are described in terms of Markush groups, those skilled in the art will recognize that the present disclosure is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided.

Provisos may apply to any of the disclosed categories or embodiments wherein any one or more of the other above disclosed embodiments or species may be excluded from such categories or embodiments.

The present disclosure further embraces isolated compounds according to Formulae (IV), (IV-C), (V), (V-A), (V-B), (V-C), or (V-D), including embodiments. The expression “isolated compound” refers to a preparation of a compound of Formulae (IV), (IV-C), (V), (V-A), (V-B), (V-C), or (V-D), including embodiments, or a mixture of compounds according to Formulae (IV), (IV-C), (V), (V-A), (V-B), (V-C), or (V-D), including embodiments, wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of Formulae (IV), (IV-C), (V), (V-A), (V-B), (V-C), or (V-D), including embodiments, or a mixture of compounds according to Formulae (IV), (IV-C), (V), (V-A), (V-B), (V-C), or (V-D), including embodiments, which contains the named compound or mixture of compounds according to Formulae (IV), (IV-C), (V), (V-A), (V-B), (V-C), or (V-D), including embodiments, in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds described herein and intermediates may be isolated from their reaction mixtures and purified by standard techniques such as filtration, liquid-liquid extraction, solid phase extraction, distillation, recrystallization or chromatography, including flash column chromatography, or HPLC.

### Isomerism and Tautomerism in Compounds Described Herein

### Tautomerism

Within the present disclosure it is to be understood that a compound of Formulae (IV), (IV-C), (V), (V-A), (V-B), (V-C), or (V-D), including embodiments, or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the present disclosure encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

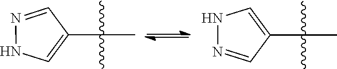 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

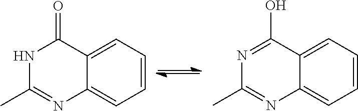

is an example of tautomerism. Accordingly, a structure depicted herein as one tautomer is intended to also include the other tautomer.

### Optical Isomerism

It will be understood that when compounds of the present disclosure contain one or more chiral centers, the compounds may exist in, and may be isolated as pure enantiomeric or diastereomeric forms or as racemic mixtures. The present disclosure therefore includes any possible enantiomers, diastereomers, racemates or mixtures thereof of the compounds described herein.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example below, the Cahn-Ingold-Prelog ranking is A>B>C>D. The lowest ranking atom, D is oriented away from the viewer.

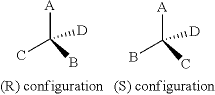

The present disclosure is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound described herein, or a chiral intermediate thereof, is separated into 99% wt. % pure optical isomers by HPLC using a suitable chiral column, such as a member of the series of DAICEL® CHIRALPAKR® family of columns (Daicel Chemical Industries, Ltd., Tokyo, Japan). The column is operated according to the manufacturer's instructions.

### Rotational Isomerism

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species (see below). It is further understood that certain structural elements, including steric bulk or substituents on the amide nitrogen, may enhance the stability of a rotamer to the extent that a compound may be isolated as, and exist indefinitely, as a single stable rotamer. The present disclosure therefore includes any possible stable rotamers of formula (I) which are biologically active in the treatment of cancer or other proliferative disease states.

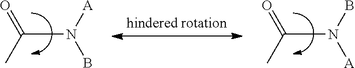

### Regioisomerism

In some embodiments, the compounds described herein have a particular spatial arrangement of substituents on the aromatic rings, which is related to the structure activity relationship demonstrated by the compound class. Often such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below.

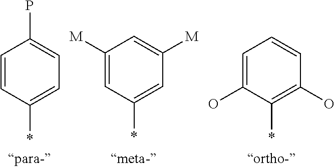

In various embodiments, the compound or set of compounds, such as are among the inventive compounds or are used in the inventive methods, can be any one of any of the combinations and/or sub-combinations of the above-listed embodiments.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties: (A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and

(B) alkyl, heteroalkyl, cycloalkyl, heterocycloalkyl, aryl, heteroaryl, substituted with at least one substituent selected from:
(i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(ii) alkyl, heteroalkyl, cycloalkyl, heterocycloalkyl, aryl, heteroaryl, substituted with at least one substituent selected from:
(a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Certain compounds of the present invention possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable moiety” as used herein refers to a moiety that can be covalently or noncovalently attached to a compound or biomolecule that can be detected for instance, using techniques known in the art. In embodiments, the detectable moiety is covalently attached. The detectable moiety may provide for imaging of the attached compound or biomolecule. The detectable moiety may indicate the contacting between two compounds. Exemplary detectable moieties are fluorophores, antibodies, reactive dies, radio-labeled moieties, magnetic contrast agents, and quantum dots. Exemplary fluorophores include fluorescein, rhodamine, GFP, coumarin, FITC, Alexa fluor, Cy3, Cy5, BODIPY, and cyanine dyes. Exemplary radionuclides include Fluorine-18, Gallium-68, and Copper-64. Exemplary magnetic contrast agents include gadolinium, iron oxide and iron platinum, and manganese.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present invention contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present invention.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Pharmaceutically acceptable excipient” and “pharmaceutically acceptable carrier” refer to a substance that aids the administration of a compound to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

An “inhibitor” or “inhibitor compound” refers to a compound (e.g., compounds described herein) that reduces the activity of a target enzyme, protein, peptide or polypeptide (e.g., a peptidase, signal peptidase (SPase), bacterial type I SPase, etc.), homolog or fragment thereof, when compared to a control, such as absence of the compound or a compound with known inactivity.

An “inhibited peptidase,” “inhibited signal peptidase (SPase),” “inhibited SPase,” “inhibited bacterial type I peptidase (SPase),” or “inhibited peptide” and the like refers to a complex comprising an enzyme (e.g., a peptidase) and an inhibitor compound. For example, the the enzyme (e.g., peptidase) and the inhibitor compound may be complexed through a bond (e.g., a covalent bond).

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. In embodiments, the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences, with or without N-terminus methionine residues; immunologically tagged proteins; and the like.

A polypeptide, or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway (e.g., MAP kinase pathway).

As defined herein, the term “activation”, “activate”, “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist. In embodiments, an agonist is a molecule that interacts with a target to cause or promote an increase in the activation of the target. In embodiments, activators are molecules that increase, activate, facilitate, enhance activation, sensitize, or up-regulate, e.g., a gene, protein, ligand, receptor, or cell.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist. An antagonist prevents, reduces, inhibits, or neutralizes the activity of an agonist, and an antagonist can also prevent, inhibit, or reduce constitutive activity of a target, e.g., a target receptor, even where there is no identified agonist. In embodiments, inhibitors are molecules that decrease, block, prevent, delay activation, inactivate, desensitize, or down-regulate, e.g., a gene, protein, ligand, receptor, or cell. An inhibitor may also be defined as a molecule that reduces, blocks, or inactivates a constitutive activity. An “antagonist” is a molecule that opposes the action(s) of an agonist.

The terms “peptidase,” “bacterial peptidase,” “signal peptidase,” “bacterial type I peptidase,” “bacterial type I signal peptidase,” and “protease” and the like refer to a protein (including homologs, isoforms, and functional fragments thereof) that performs proteolysis, protein catabolism, by hydrolysis of peptide bonds. The terms includes any recombinant or naturally-occurring forms or variants thereof that maintain biological activity and function (e.g. within at least 30%, 40%, 50%, 60%, 70%, 80%, 90%, 95%, or 100% activity compared to wildtype peptidases). The term includes any mutant form of peptidase variants (e.g., frameshift mutations) thereof that maintain peptidase activity (e.g. within at least 30%, 40%, 50%, 60%, 70%, 80%, 90%, 95%, or 100% activity compared to wildtype peptidase).

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including MDS, AML, ALL, ATLL and CML), or multiple myeloma.

As used herein, the terms “infection” or “bacterial infection” refer to a disease or condition characterized by invasion of an organism's body tissues by disease-causing agents (e.g., pathogenic bacteria), their multiplication, and the reaction of host tissues to the infectious agents and the toxins they produce. Infectious disease, also known as transmissible disease or communicable disease, is illness resulting from an infection. A bacterial infection may be caused by Gram-positive or Gram-negative bacteria. Non-limiting examples of bacteria that may cause an infection include Elizabethkingia meningoseptica, Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

Each species of bacteria has specific effect and causes symptoms in patients who are infected. For instance, symptoms of a bacterial infection may include localized redness, heat, swelling and pain at the site of the infection. Bacterial throat pain is often characterized by more pain on one side of the throat. Bacterial infections can become a systemic inflammatory response resulting in massive vasodilation, shock, and death.

The terms “treating” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. As indicated above, the prevention may be complete (no detectable symptoms) or partial, such that fewer symptoms are observed than would likely occur absent treatment. In embodiments, prevent refers to slowing the progression of the disease, disorder or condition or inhibiting progression thereof to a harmful or otherwise undesired state.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). The therapeutically effective amount can be ascertained by measuring relevant physiological effects, and it can be adjusted in connection with the dosing regimen and diagnostic analysis of the subject's condition, and the like. By way of example, measurement of the serum level of an inhibitor (or, e.g., a metabolite thereof) at a particular time post-administration may be indicative of whether a therapeutically effective amount has been administered.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Adjusting the dose to achieve maximal therapeutic window efficacy or toxicity in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent, chemotherapeutic, or treatment for a neurodegenerative disease). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

By “co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). The compositions of the present invention can be delivered transdermally, by a topical route, or formulated as applicator sticks, solutions, suspensions, emulsions, gels, creams, ointments, pastes, jellies, paints, powders, and aerosols.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating cancer (e.g. colon cancer), cardiovascular disease, metabolic disease, immune or inflammatory disease or disorder.

In some embodiments, co-administration includes administering one active agent within 0.5, 1, 2, 4, 6, 8, 10, 12, 16, 20, 24 hours, 2 days, 4 days, 1 week or 1 month of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for infections (e.g. bacterial infections), inflammation, and/or vasodilation.

“Anti-inflammatory agent” is used in accordance with its plain ordinary meaning and refers to a composition (e.g. compound, drug, antagonist, inhibitor, modulator) used in any way to reduce inflammation or swelling. In some embodiments, an anti-inflammatory agent is an agent identified herein having utility in methods of treating an inflammatory disease or disorder. In some embodiments, an anti-inflammatory agent is an agent approved by the FDA or similar regulatory agency of a country other than the USA, for reducing swelling and inflammation.

The compounds described herein can be administered to treat an immune or inflammatory disease or disorder, a cardiovascular or metabolic disease or disorder and/or infection. In this regard, the compounds disclosed herein may be administered either alone to treat such diseases or disorders or may be co-administered with another therapeutic agent to treat such diseases or disorders.

The compounds disclosed herein may be co-administered with a cytokine or agonist or antagonist of cytokine function, (including agents which act on cytokine signaling pathways such as modulators of the SOCS system) including alpha-, beta-, and gamma-interferons; insulin-like growth factor type I (IGF-1); interleukins (IL) including IL1 to 17, and interleukin antagonists or inhibitors such as analcinra; tumour necrosis factor alpha (TNF-.alpha.) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

The compounds disclosed herein may be co-administered with an anti-inflammatory agent, such as thalidomide or a derivative thereof, a retinoid, dithranol or calcipotriol, a non-steroidal anti-inflammatory agent (hereinafter NSAID) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The compounds disclosed herein may be co-administered with a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

“Antibiotic” is used in accordance with its plain ordinary meaning and refers to a composition (e.g. compound, drug, antagonist, inhibitor, modulator) having antimicrobial activity used in the treatment and prevention of bacterial infections. An antibiotic may either kill or inhibit the growth of bacteria. An antibiotic may also possess antiprotozoal activity.

Additionally, the compounds described herein can be co-administered with conventional antibiotic agents including, but not limited to, ceftriaxone, meropenem, ceftazidime, cefepime, cefotaxime, piperacillin and/or tazobactam, ampicillin and/or sulbactam, imipenem and/or cilastatin, levofloxacin, or clindamycin.

The inhibitors (e.g., peptidase inhibitors) disclosed herein can be administered by any acceptable route, such oral, intraadiposal, intraarterial, intraarticular, intracranial, intradermal, intralesional, intramusculay, intranasal, intraocularal, intrapericardial, intraperitoneal, intrapleural, intraprostatical, intrarectal, intrathecal, intratracheal, intratumoral, intraumbilical, intravaginal, intravenousl, intravesicullar, intravitreal, liposomal, local, mucosal, parenteral, rectal, subconjunctival, subcutaneous, sublingual, topical, transbuccal, transdermal, vaginal, in cremes, in lipid compositions, via a catheter, via a lavage, via continuous infusion, via infusion, via inhalation, via injection, via local delivery, via localized perfusion, bathing target cells directly, or any combination thereof.

The inhibitors (e.g., peptidase inhibitors) disclosed herein may be administered once daily until study reached endpoint. The inhibitors disclosed herein may be administered at least three times but in some studies four or more times depending on the length of the study and/or the design of the study.

The methods disclosed herein may be used in combination with additional antimicrobial therapy or therapy to treat the symptoms of infection (e.g., pain and inflammation). In some embodiments, the additional therapy comprises surgery, intubation, dialysis, fluid replacement, insertion of a central venous catheter, and/or administration of an antihypotensive and/or anti-inflammatory agent. In some embodiments, the infection is caused by a resistant or mutant bacteria such as methicillin-resistant (MRSA) bacteria, vancomycin-intermediate (VISA) bacteria, vancomycin-resistant Staphylococcus aureus (VRSA), multidrug-resistant (MDR) bacteria, pandrug-resistant (PDR) Gram-negative bacteria, or extensively drug-resistant (XDR) bacteria.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulator” refers to a composition that increases or decreases the level of a target molecule or the function of a target molecule or the physical state of the target of the molecule. In some embodiments, a modulator is a compound that reduces the severity of one or more symptoms of a disease (e.g. infectious disease) associated with an enzyme (e.g., a peptidase). A peptidase modulator is a compound that increases or decreases the activity or function or level of activity or level of function of a peptidase. A modulator may act alone, or it may use a cofactor, e.g., a protein, metal ion, or small molecule. Examples of modulators include small molecule compounds and other bioorganic molecules.

Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an activator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule. In embodiments, the terms “modulate,” “modulation” and the like refer to the ability of a molecule (e.g., an activator or an inhibitor) to increase or decrease the function or activity of an enzyme (e.g., peptidase), either directly or indirectly, relative to the absence of the molecule.

The term “associated” or “associated with” in the context of a substance or substance activity or function associated with a disease (e.g. a protein associated disease, a cancer associated with peptidase activity (e.g., infectious disease) means that the disease (e.g. infectious disease) is caused by (in whole or in part), or a symptom of the disease is caused by (in whole or in part) the substance or substance activity or function. For example, an infectious disease associated with peptidase activity or function may be an infectious disease that results (entirely or partially) from aberrant peptidase function (e.g. enzyme activity, protein-protein interaction, signaling pathway) wherein a particular symptom of the disease is caused (entirely or partially) by aberrant peptidase activity or function. As used herein, what is described as being associated with a disease, if a causative agent, could be a target for treatment of the disease. For example, an infectious disease associated with peptidase activity or function or a peptidase associated disease (e.g., infectious disease), may be treated with a compound described herein (e.g., peptidase modulator or peptidase inhibitor), in the instance where increased peptidase activity or function (e.g. signaling pathway activity) causes the disease (e.g., infectious disease). For example, an infectious disease associated with peptidase activity or function or an peptidase associated infectious disease, may be treated with a peptidase modulator or peptidase inhibitor, in the instance where increased peptidase activity or function (e.g. signaling pathway activity) causes the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

For example, binding of a peptidase with a compound as described herein may reduce the level of a product of the peptidase catalyzed reaction or the level of a downstream derivative of the product or binding may reduce the interactions between the peptidase or a reaction product and downstream effectors or signaling pathway components, resulting in changes in cell (e.g., bacterial cell) growth, proliferation, or survival.

As used herein, the terms “peptidase inhibitor,” “peptidase antagonist,” “SPase inhibitor,” “SPase antagonist” and all other related art-accepted terms, many of which are set forth below, refer to a compound capable of modulating, either directly or indirectly, the peptidase or enzyme in an in vitro assay, an in vivo model, and/or other means indicative of therapeutic efficacy. The terms also refer to a compound that exhibits at least some therapeutic benefit in a human subject.

The phrase “in a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term “proliferative activity” encompasses an activity that promotes, that is necessary for, or that is specifically associated with, for example, normal cell division, as well as cancer, tumors, dysplasia, cell transformation, metastasis, and angiogenesis.

“Substantially pure” indicates that a component makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition (percentage in a weight per weight basis).

The terms “specifically binds” and “selectively binds”, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two-fold greater, at least 10-times greater, at least 20-times greater, or at least 100-times greater than the affinity with any other antibody, or binding composition derived therefrom. In embodiments, the antibody will have an affinity that is greater than about 10⁹ liters/mol, as determined by, e.g., Scatchard analysis (Munsen, et al. (1980) Analyt. Biochem. 107:220-239).

The terms “DNA”, “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

As used herein, the terms “variants” and “homologs” are used interchangeably to refer to amino acid or nucleic acid sequences that are similar to reference amino acid or nucleic acid sequences, respectively. The term encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Thus, variants and homologs encompass naturally occurring amino acid and nucleic acid sequences encoded thereby and their isoforms, as well as splice variants of a protein or gene. The terms also encompass nucleic acid sequences that vary in one or more bases from a naturally-occurring nucleic acid sequence but still translate into an amino acid sequence that corresponds to the naturally-occurring protein due to degeneracy of the genetic code. Non-naturally-occurring variants and homologs include polypeptides and nucleic acids that comprise a change in amino acid or nucleotide sequence, respectively, where the change in sequence is artificially introduced (e.g., muteins); for example, the change is generated in the laboratory by human intervention (“hand of man”). Therefore, non-naturally occurring variants and homologs may also refer to those that differ from the naturally-occurring sequences by one or more conservative substitutions and/or tags and/or conjugates.

The term “muteins” as used herein refers broadly to mutated recombinant proteins. These proteins usually carry single or multiple amino acid substitutions and are frequently derived from cloned genes that have been subjected to site-directed or random mutagenesis, or from completely synthetic genes.

### II. Compositions

In an aspect provided herein, is an inhibited peptidase, comprising a signal peptidase (SPase) inhibitor having a bond to an amino acid residue of a bacterial type I SPase, a bacterial type I SPase homolog, or a bacterial type I SPase lysine homolog.

In embodiments, the inhibitor forms an irreversible bond with the amino acid residue. In embodiments, the bond is a covalent bond.

In embodiments, the peptidase comprises region B, region C, region C′, and region D. B comprises amino acid sequence PSXSMXPTLX (SEQ ID NO: 1). C comprises amino acid sequence DXIXVXKXX (SEQ ID NO: 2). C′ comprises amino acid sequence RGDXXVFXXP (SEQ ID NO: 3). D comprises amino acid sequence Y/F, I/V, KRXXGXXGD (SEQ ID NO: 4). X is any natural amino acid residue or any unnatural amino acid residue.

In embodiments, the inhibitor and the peptidase are bonded at region D of the peptidase.

In embodiments, the bond is formed between a portion of an electrophilic acceptor moiety on the inhibitor and a portion of the amino acid residue of the peptidase.

In embodiments, the electrophilic acceptor moiety on the inhibitor is a —C≡N group.

In embodiments, the inhibitor forms a bond to a lysine residue of the peptidase. In embodiments, the inhibitor forms a bond to a nitrogen atom of the side chain of the lysine residue.

In embodiments, the inhibitor is an arylomycin derivative or a bacterial signal peptide, or fragment or homolog thereof.

In embodiments, the inhibited peptidase has structural Formula (I):

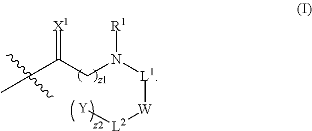

X¹ is ═O or ═NH. R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. W is a substituted or unsubstituted linear peptide or a substituted or unsubstituted cyclic peptide, wherein the peptide comprises at least three of: natural amino acid residues, unnatural amino acid residues, or a combination thereof. L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof. R^{1A}, R^{1B}, R^{1C}, and R^{1D} are independently hydrogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{1B} and R^{1C} substituents bonded to the same nitrogen atom may optionally be joined to form a substituted or unsubstituted heterocycloalkyl or substituted or unsubstituted heteroaryl. The symbol “ [symbol] ” indicates the point of attachment between the peptidase and the inhibitor. The symbol n1 is an integer from 0 to 4. The symbols m1 and v1 are independently 1 or 2. The symbol z1 is an integer from 1 to 4. The symbol z2 is 0 or 1.

In embodiments, the inhibited peptidase has structural Formula (I-A):

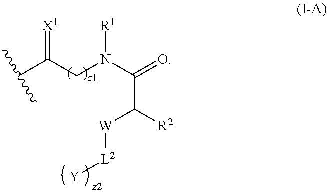

The symbol “ [symbol] ” z1, z2, X¹, R¹, W, Y, and L² are as described herein, including embodiments. R² is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, X¹ is ═NH.

In embodiments, W comprises at least one amino acid selected from 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

In embodiments, W comprises glycine and at least one amino acid selected from: 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

In embodiments, the inhibited peptidase is:

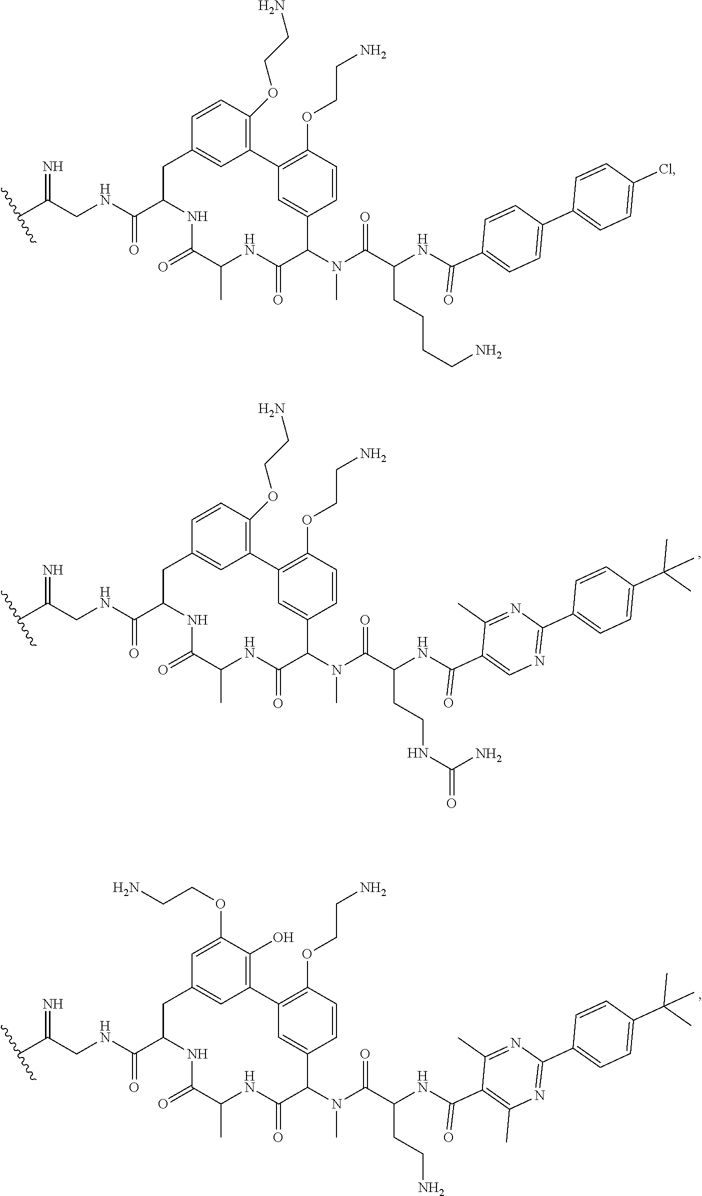 [SMILES: *C(=N)CNC(=O)C1Cc2ccc(OCCN)c(c2)-c2cc(ccc2OCCN)C(N(C)C(=O)C(CCCCN)NC(=O)c2ccc(-c3ccc(Cl)cc3)cc2)C(=O)NC(C)C(=O)N1 ; *C(=N)CNC(=O)C1Cc2ccc(OCCN)c(c2)-c2cc(ccc2OCCN)C(N(C)C(=O)C(CCNC(N)=O)NC(=O)c2cnc(-c3ccc(C(C)(C)C)cc3)nc2C)C(=O)NC(C)C(=O)N1 ; *C(=N)CNC(=O)C1Cc2cc(OCCN)c(O)c(c2)-c2cc(ccc2OCCN)C(N(C)C(=O)C(CCN)NC(=O)c2c(C)nc(-c3ccc(C(C)(C)C)cc3)nc2C)C(=O)NC(C)C(=O)N1]

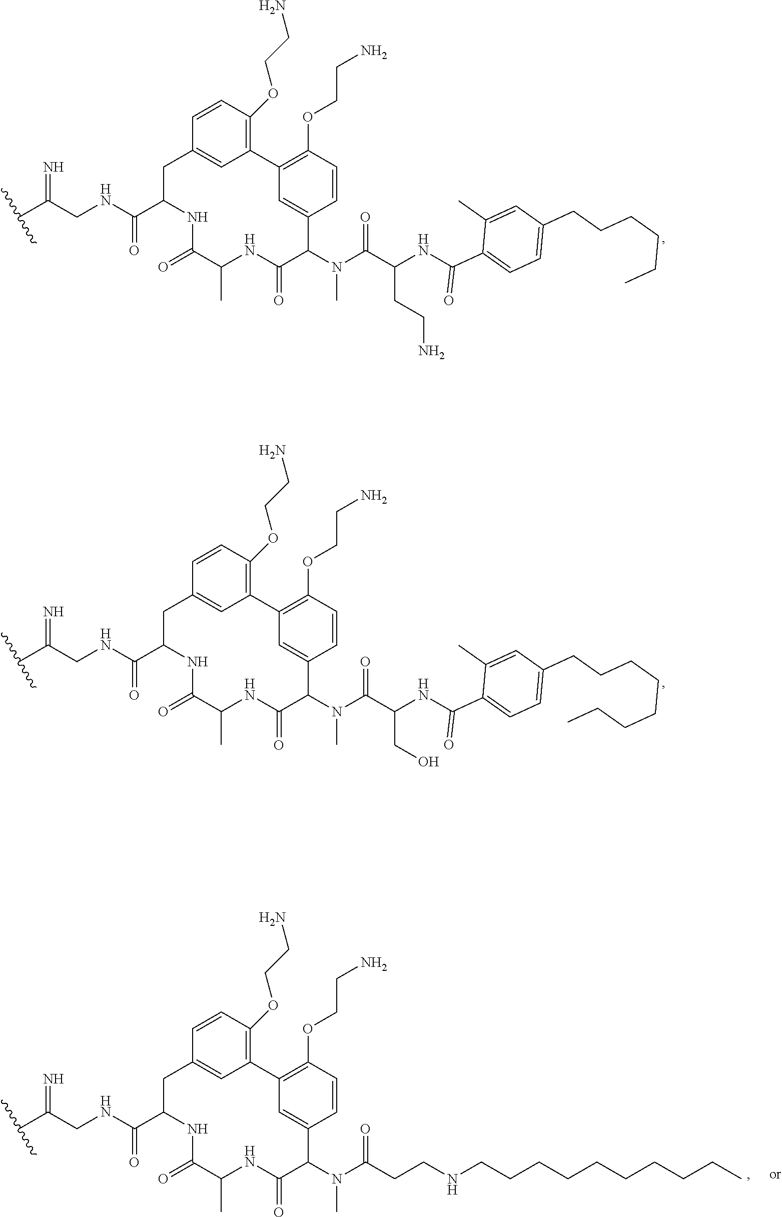 [SMILES: *C(=N)CNC(=O)C1Cc2ccc(OCCN)c(c2)-c2cc(ccc2OCCN)C(N(C)C(=O)C(CCN)NC(=O)c2ccc(CCCCCC)cc2C)C(=O)NC(C)C(=O)N1 ; *C(=N)CNC(=O)C1Cc2ccc(OCCN)c(c2)-c2cc(ccc2OCCN)C(N(C)C(=O)C(CO)NC(=O)c2ccc(CCCCCCCC)cc2C)C(=O)NC(C)C(=O)N1 ; *C(=N)CNC(=O)C1Cc2ccc(OCCN)c(c2)-c2cc(ccc2OCCN)C(N(C)C(=O)CCNCCCCCCCCCC)C(=O)NC(C)C(=O)N1]

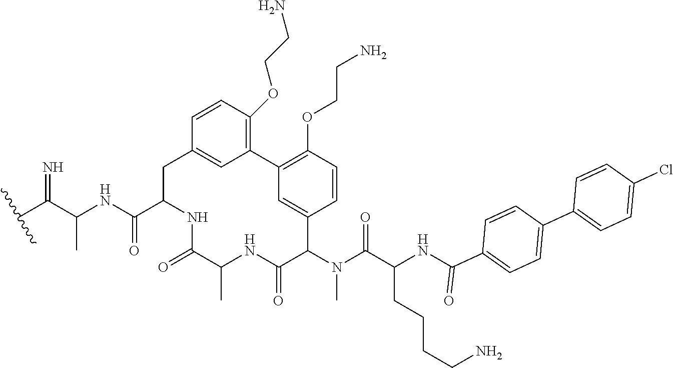 [SMILES: *C(=N)C(C)NC(=O)C1Cc2ccc(OCCN)c(c2)-c2cc(ccc2OCCN)C(N(C)C(=O)C(CCCCN)NC(=O)c2ccc(-c3ccc(Cl)cc3)cc2)C(=O)NC(C)C(=O)N1]

where the symbol “ [symbol] ” indicates the point of attachment between the peptidase and the inhibitor.

In embodiments, the inhibited peptidase has structural Formula (I-B):

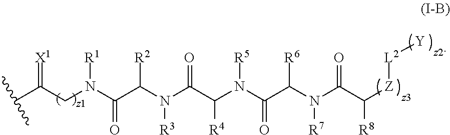

The symbol “ [symbol] ,” z1, z2, X¹, R¹, R², Y, and L² are as described herein, including embodiments. The symbol z3 is an integer from 0 to 20. R⁴ and R⁶ are independently hydrogen, halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² and R⁶; or R² and R⁸ may optionally be joined to form a substituted or unsubstituted heterocycloalkyl. R³, R⁵, and R⁷ are independently hydrogen, halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl-C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl-C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H) [optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl. Z is independently a substituted or unsubstituted natural amino acid or a substituted or unsubstituted unnatural amino acid. In embodiments, R² and a Z amino acid; R⁴ and a Z amino acid; R⁶ and a Z amino acid; or two Z amino acids may optionally be joined by a linking moiety, L³, to form a substituted or unsubstituted heterocycloalkyl. L³ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R^{8A} is independently hydrogen or —(C₁-C₆)alkyl. R^{8B} and R^{8C} are independently hydrogen or optionally substituted —(C₁-C₆)alkyl or R^{8B} and R^{8C} and the nitrogen atom to which they are attached optionally form a heterocycloalkyl ring. R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R¹⁴)₂, —C(O)N(R¹⁴)₂, or —SO₂N(R¹⁴)₂. In embodiments, R^{8D} and R^{8E} and the nitrogen atom to which they are attached optionally form a heterocycloalkyl ring. R¹⁴ is independently hydrogen or —(C₁-C₆)alkyl. In embodiments, two R¹⁴ groups and the nitrogen atom to which they are attached optionally form a heterocycloalkyl ring

In embodiments, R² and R⁶ are joined to form a substituted or unsubstituted heterocycloalkyl.

In embodiments, z3 is 0.

In embodiments, Z comprises at least one amino acid selected from 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

In embodiments, Z comprises glycine and at least one amino acid selected from: 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid. In embodiments, Z comprises glycine.

In embodiments, the inhibited peptidase has structural Formula (III):

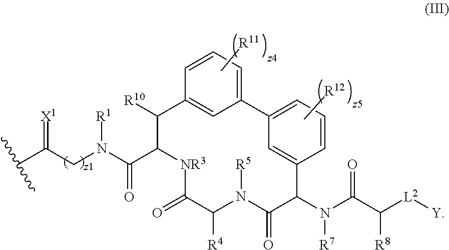

The symbol “ [symbol] ,” z1, X¹, R¹, R³, R⁴, R⁵, R⁷, R⁸, Y, and L² are as described herein, including embodiments. The symbols z4 and z5 are independently an integer from 0 to 4. R¹⁰ is hydrogen, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R″ is hydrogen,

halogen, —CCl₃, —CBr₃, —CF₃, —CI₃, —CN, —OR^{2A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHC₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹² is hydrogen,
halogen, —CCl₃, —CBr₃, —CF₃, —CI₃, —CN, —OR^{3A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the inhibited peptidase has structural Formula (III-A):

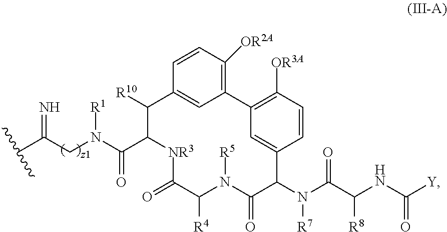

The symbol “ [symbol] ,” z1, R¹, R³, R⁴, R⁵, R⁷, R⁸, R¹⁰, R^{2A}, R^{3A}, and Y are as described herein, including embodiments. In embodiments, R^{2A} and R^{3A} are independently hydrogen, substituted or unsubstituted alkyl, or substituted or unsubstituted heteroalkyl.

In embodiments, Y is substituted or unsubstituted aryl or substituted or unsubstituted heteroaryl.

In embodiments, the inhibited peptidase has structural Formula (III-B):

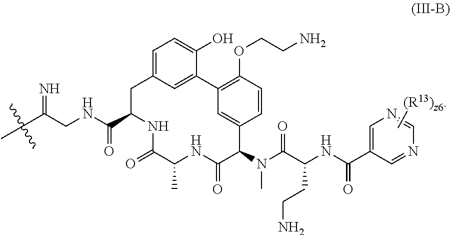

The symbol z6 is an integer from 0 to 3. R¹³ is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{2A} and R^{3A} are independently substituted or unsubstituted heteroalkyl.

In embodiments, the inhibited peptidase has structural Formula (III-C):

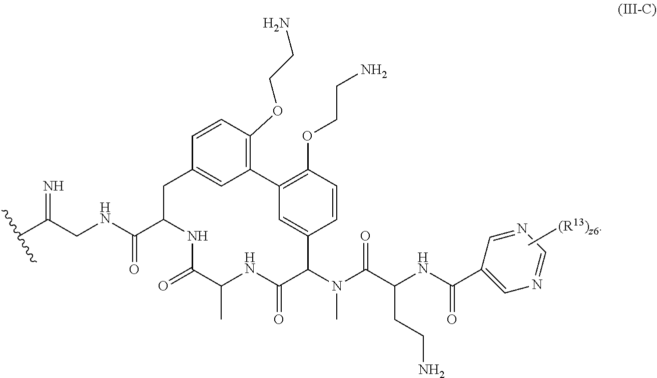

The symbol “ [symbol] ” z6, and R¹³ are as described herein, including embodiments.

In embodiments, R¹³ is independently substituted or unsubstituted alkyl, substituted or unsubstituted aryl, or substituted or unsubstituted heteroaryl.

In embodiments, the inhibited peptidase has structural Formula (III-D):

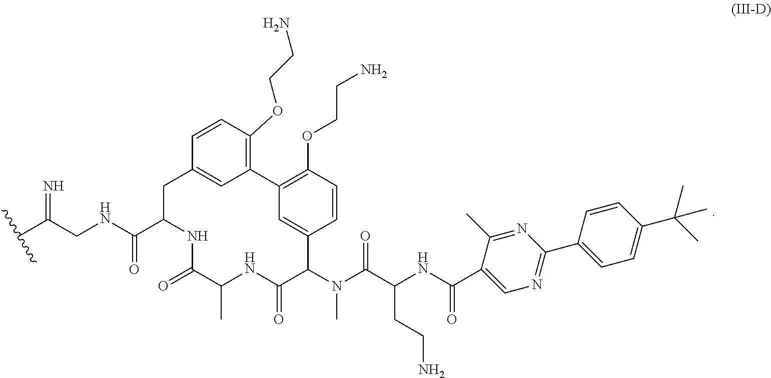 [SMILES: *C(=N)CNC(=O)C1Cc2ccc(OCCN)c(c2)-c2cc(ccc2OCCN)C(N(C)C(=O)C(CCN)NC(=O)c2cnc(-c3ccc(C(C)(C)C)cc3)nc2C)C(=O)NC(C)C(=O)N1]

The symbol “ [symbol] ” is as described herein.

In embodiments, the inhibited peptidase is in a bacterial cell.

In embodiments, the amino acid is Lys¹⁴⁶ of E. coli.

In embodiments, the signal peptidase is a Gram-positive a signal peptidase or a Gram-negative a signal peptidase. In embodiments, the signal peptidase is a Gram-negative a signal peptidase. In embodiments, the signal peptidase is LepB.

In an aspect provided herein, is an inhibited peptide, comprising a serine-lysine catalytic dyad or a serine-serine lysine catalytic triad; and a peptide inhibitor having a bond to an amino group of the lysine. In embodiments, the bond is irreversible. In embodiments, the bond is a covalent bond.

In embodiments, the inhibited peptide is a bacterial peptide or a mammalian peptide. In embodiments, the inhibited peptide is selected from bacterial UmuD, bacterial LexA, bacterial Lon protease, bacterial signal peptidase, bacterial penicillin binding protein V, bacterial penicillin binding protein 1a, bacterial penicillin binding protein 1b, bacterial penicillin binding protein 2; bacterial penicillin binding protein 3; mammalian lactoferrin; mammalian mitochondrial signal peptidase; N-terminal Serine or Threonine protease; bacterial penicillin G acylase precursor; mammalian glycosylasparaginase precursor, and a bacterial penicillin binding proteins homologous to E. coli PBP1a, 1b, 2, 3, 4, 5, or 6. In embodiments, the inhibited bacterial peptide is selected from Escherichia coli UmuD, Escherichia coli LexA, Escherichia coli Lon protease, Escherichia coli signal peptidase, Escherichia coli penicillin binding protein V; Escherichia coli penicillin binding protein 1a, Escherichia coli penicillin binding protein 1b, Escherichia coli penicillin binding protein 2, Escherichia coli penicillin binding protein 3, Homo sapiens lactoferrin, Homo sapiens mitochondrial signal peptidase, N-terminal Serine or Threonine protease, Escherichia coli penicillin G acylase precursor, Homo sapiens glycosylasparaginase precursor, and an Escherichia coli penicillin binding proteins homologous to E. coli PBP1a, 1b, 2, 3, 4, 5, or 6.

In embodiments, the inhibited peptide has structural Formula (I):

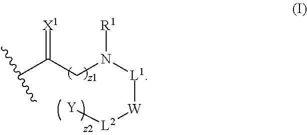

X¹ is ═O or ═NH. R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. W is a substituted or unsubstituted linear peptide or a substituted or unsubstituted cyclic peptide, wherein the peptide comprises at least three of: natural amino acid residues, unnatural amino acid residues, or a combination thereof. L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Y is —SOₙ₁R^{1A}, —SOᵥNR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof. R^{1A}, R^{1B}, R^{1C}, and R^{1D} are independently hydrogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{1B} and R^{1C} substituents bonded to the same nitrogen atom may optionally be joined to form a substituted or unsubstituted heterocycloalkyl or substituted or unsubstituted heteroaryl. The symbol “ [symbol] ” indicates the point of attachment between the peptide and the inhibitor. The symbol n1 is an integer from 0 to 4. The symbols m1 and v1 are independently 1 or 2. The symbol z1 is an integer from 1 to 4. The symbol z2 is 0 or 1.

In embodiments, the inhibited peptide has structural Formula (I-A):

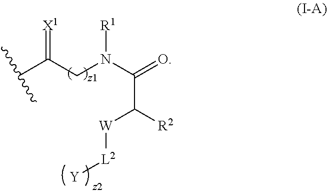

The symbol “ [symbol] ” z1, z2, X¹, R¹, W, Y, and L² are as described herein, including embodiments. R² is independently hydrogen,
halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, X¹ is ═NH.

In embodiments, W comprises at least one amino acid selected from 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

In embodiments, W comprises glycine and at least one amino acid selected from: 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

In embodiments, the inhibited peptide is:

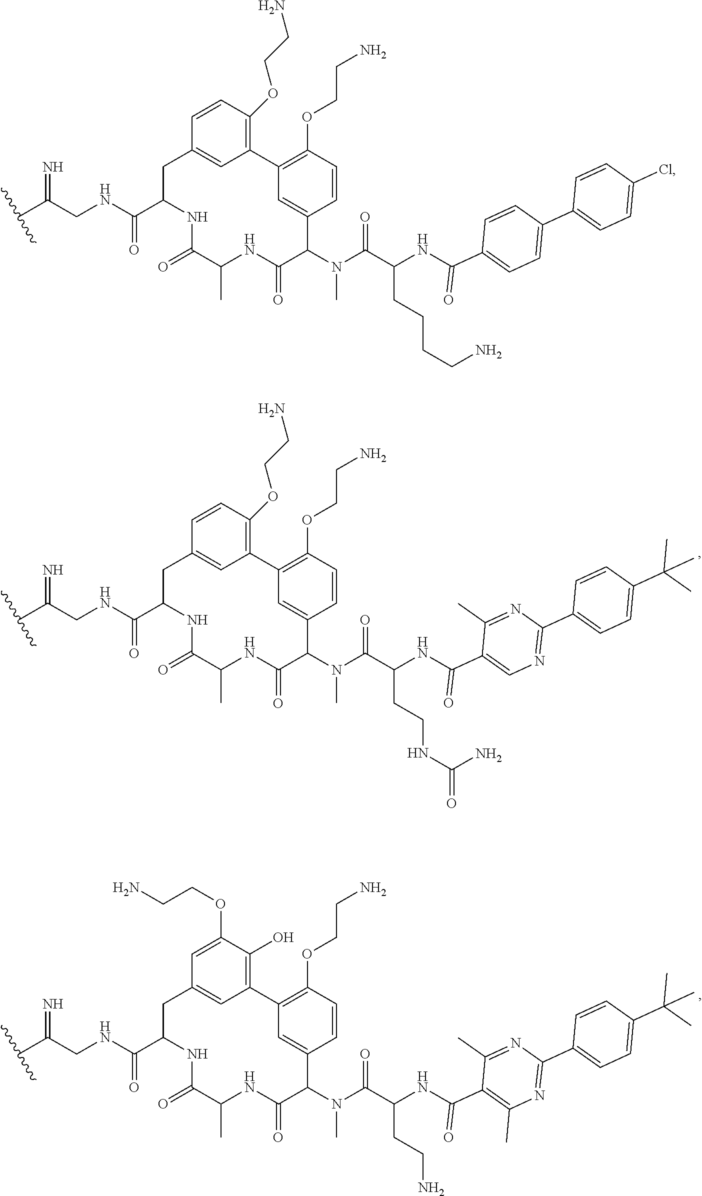 [SMILES: *C(=N)CNC(=O)C1Cc2ccc(OCCN)c(c2)-c2cc(ccc2OCCN)C(N(C)C(=O)C(CCCCN)NC(=O)c2ccc(-c3ccc(Cl)cc3)cc2)C(=O)NC(C)C(=O)N1 ; *C(=N)CNC(=O)C1Cc2ccc(OCCN)c(c2)-c2cc(ccc2OCCN)C(N(C)C(=O)C(CCNC(N)=O)NC(=O)c2cnc(-c3ccc(C(C)(C)C)cc3)nc2C)C(=O)NC(C)C(=O)N1 ; *C(=N)CNC(=O)C1Cc2cc(OCCN)c(O)c(c2)-c2cc(ccc2OCCN)C(N(C)C(=O)C(CCN)NC(=O)c2c(C)nc(-c3ccc(C(C)(C)C)cc3)nc2C)C(=O)NC(C)C(=O)N1]

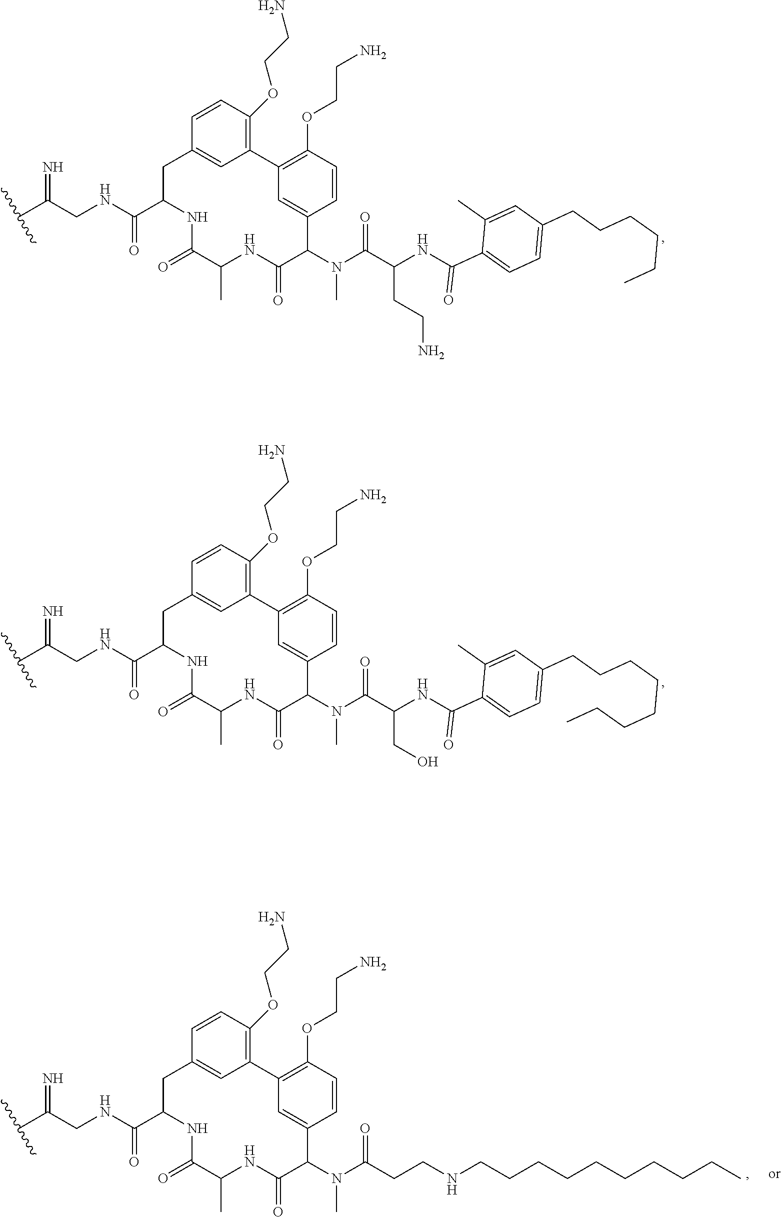 [SMILES: *C(=N)CNC(=O)C1Cc2ccc(OCCN)c(c2)-c2cc(ccc2OCCN)C(N(C)C(=O)C(CCN)NC(=O)c2ccc(CCCCCC)cc2C)C(=O)NC(C)C(=O)N1 ; *C(=N)CNC(=O)C1Cc2ccc(OCCN)c(c2)-c2cc(ccc2OCCN)C(N(C)C(=O)C(CO)NC(=O)c2ccc(CCCCCCCC)cc2C)C(=O)NC(C)C(=O)N1 ; *C(=N)CNC(=O)C1Cc2ccc(OCCN)c(c2)-c2cc(ccc2OCCN)C(N(C)C(=O)CCNCCCCCCCCCC)C(=O)NC(C)C(=O)N1]

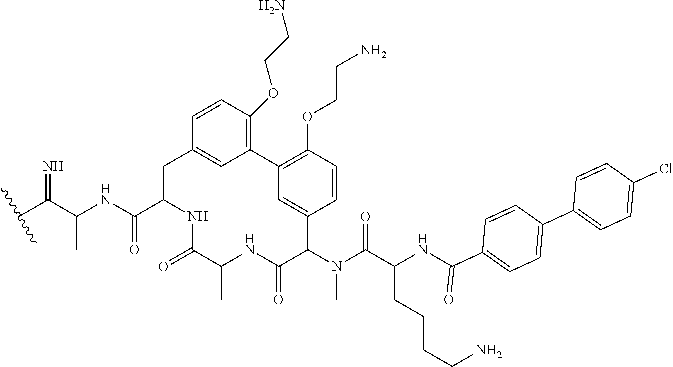 [SMILES: *C(=N)C(C)NC(=O)C1Cc2ccc(OCCN)c(c2)-c2cc(ccc2OCCN)C(N(C)C(=O)C(CCCCN)NC(=O)c2ccc(-c3ccc(Cl)cc3)cc2)C(=O)NC(C)C(=O)N1]

where the symbol “ [symbol] ” indicates the point of attachment between the peptide and the inhibitor.

In embodiments, the inhibited peptide has structural Formula (I-B):

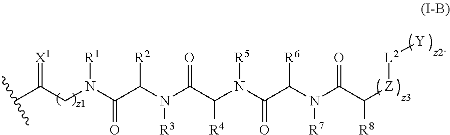

The symbol “ [symbol] ”, z1, z2, X¹, R¹, R², Y, and L² are as described herein, including embodiments. The symbol z3 is an integer from 0 to 20. R⁴ and R⁶ are independently hydrogen,

halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² and R⁶; or R² and R⁸ may optionally be joined to form a substituted or unsubstituted heterocycloalkyl. R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl-C(O)OR^{8A}, —(C₁-C₆)alkyl-NR^{8D}R^{8E}, —(C₁-C₆)alkyl-NR^{8A}OR^{A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl-C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H) [optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl. Z is independently a substituted or unsubstituted natural amino acid or a substituted or unsubstituted unnatural amino acid. In embodiments, R² and a Z amino acid; R⁴ and a Z amino acid; R⁶ and a Z amino acid; or two Z amino acids may optionally be joined by a linking moiety, L³, to form a substituted or unsubstituted heterocycloalkyl. L³ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R^{8A} is independently hydrogen or —(C₁-C₆)alkyl. R^{8B} and R^{8C} are independently hydrogen or optionally substituted —(C₁-C₆)alkyl or R^{8B} and R^{8C} and the nitrogen atom to which they are attached optionally form a heterocycloalkyl ring. R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R¹⁴)₂, —C(O)N(R¹⁴)₂, or —SO₂N(R¹⁴)₂. In embodiments, R^{8D} and R^{8E} and the nitrogen atom to which they are attached optionally form a heterocycloalkyl ring. R¹⁴ is independently hydrogen or —(C₁-C₆)alkyl. In embodiments, two R¹⁴ groups and the nitrogen atom to which they are attached optionally form a heterocycloalkyl ring

In embodiments, R² and R⁶ are joined to form a substituted or unsubstituted heterocycloalkyl.

In embodiments, z3 is 0.

In embodiments, Z comprises at least one amino acid selected from 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

In embodiments, Z comprises glycine and at least one amino acid selected from: 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid. In embodiments, Z comprises glycine.

In embodiments, the inhibited peptide has structural Formula (III):

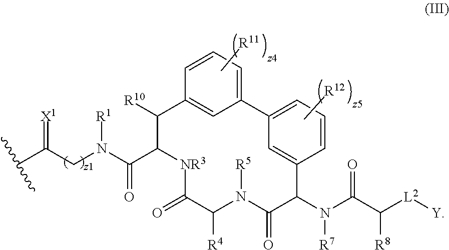

The symbol “ [symbol] ,” z1, X¹, R¹, R³, R⁴, R⁵, R⁷, R⁸, Y, and L² are as described herein, including embodiments. The symbols z4 and z5 are independently an integer from 0 to 4. R¹⁰ is hydrogen,

halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R″ is hydrogen,
halogen, —CCl₃, —CBr₃, —CF₃, —CI₃, —CN, —OR^{2A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹² is hydrogen,
halogen, —CCl₃, —CBr₃, —CF₃, —CI₃, —CN, —OR^{3A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the inhibited peptide has structural Formula (III-A):

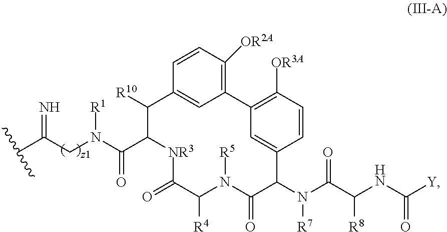

The symbol “ [symbol] ,” z1, R¹, R³, R⁴, R⁵, R⁷, R⁸, R¹⁰, R^{2A}, R^{3A}, and Y are as described herein, including embodiments. In embodiments, R^{2A} and R^{3A} are independently hydrogen, substituted or unsubstituted alkyl, or substituted or unsubstituted heteroalkyl.

In embodiments, Y is substituted or unsubstituted aryl or substituted or unsubstituted heteroaryl.

In embodiments, the inhibited peptide has structural Formula (III-B):

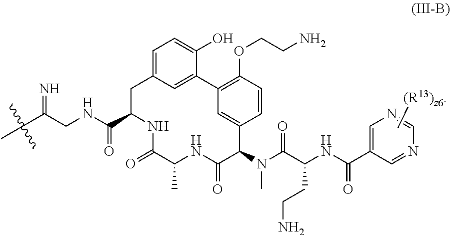

The symbol z6 is an integer from 0 to 3. R¹³ is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{2A} and R^{3A} are independently substituted or unsubstituted heteroalkyl.

In embodiments, the inhibited peptide has structural Formula (III-C):

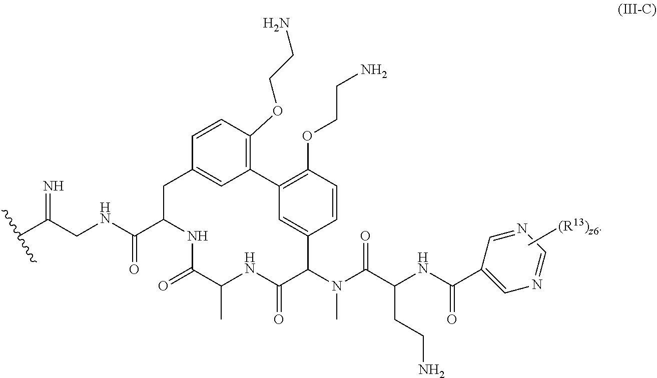

The symbol “ [symbol] ,” z6 and R¹³ are as described herein, including embodiments.

In embodiments, R¹³ is independently substituted or unsubstituted alkyl, substituted or unsubstituted aryl, or substituted or unsubstituted heteroaryl.

In embodiments, the inhibited peptide has structural Formula (III-D):

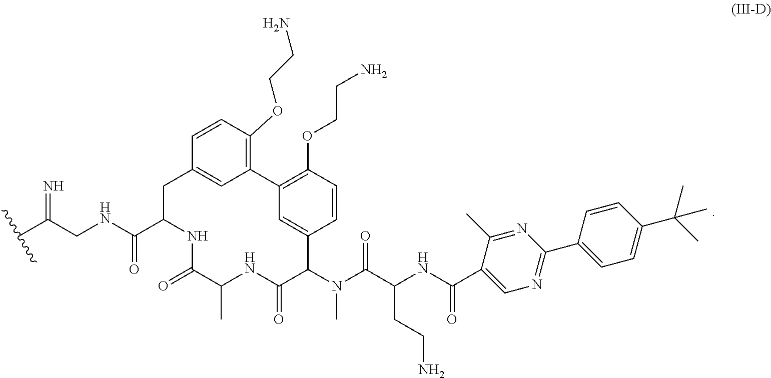 [SMILES: *C(=N)CNC(=O)C1Cc2ccc(OCCN)c(c2)-c2cc(ccc2OCCN)C(N(C)C(=O)C(CCN)NC(=O)c2cnc(-c3ccc(C(C)(C)C)cc3)nc2C)C(=O)NC(C)C(=O)N1]

The symbol is “ [symbol] ” is s d herein.

In embodiments, X¹ is ═0. In embodiments, X¹ is ═NH.

In embodiments, n1 is 0. In embodiments, n1 is 1. In embodiments, n1 is 2. In embodiments, n1 is 3. In embodiments, n1 is 4.

In embodiments, m1 is 1. In embodiments, m1 is 2. In embodiments, v1 is 1. In embodiments, v1 is 2.

In embodiments, z is 0. In embodiments, z is 1. In embodiments, z is 2. In embodiments, z is 3. In embodiments, z1 is 4.

In embodiments, z2 is 0. In embodiments, z2 is 1.

In embodiments, z3 is 0. In embodiments, z3 is 1. In embodiments, z3 is 2. In embodiments, z3 is 3. In embodiments, z3 is 4. In embodiments, z3 is 5. In embodiments, z3 is 6. In embodiments, z3 is 7.

In embodiments, z3 is 8. In embodiments, z3 is 9. In embodiments, z3 is 10. In embodiments, z3 is 11.

In embodiments, z3 is 12. In embodiments, z3 is 13. In embodiments, z3 is 14. In embodiments, z3 is 15.

In embodiments, z3 is 16. In embodiments, z3 is 17. In embodiments, z3 is 18. In embodiments, z3 is 19.

In embodiments, z3 is 20.

In embodiments, z4 is 0. In embodiments, z4 is 1. In embodiments, z4 is 2. In embodiments, z4 is 3. In embodiments, z4 is 4.

In embodiments, z5 is 0. In embodiments, z5 is 1. In embodiments, z5 is 2. In embodiments, z5 is 3. In embodiments, z5 is 4.

In embodiments, z6 is 0. In embodiments, z6 is 1. In embodiments, z6 is 2. In embodiments, z6 is 3.

### III. Methods

In an aspect provided herein, is a method of inhibiting a bacterial peptidase, comprising contacting a bacterial cell with a compound of structural Formula (IV):

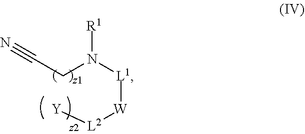

or a pharmaceutically acceptable salt thereof. R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. W is a substituted or unsubstituted linear peptide or a substituted or unsubstituted cyclic peptide, wherein the peptide comprises at least three of: natural amino acid residues, unnatural amino acid residues, or a combination thereof. L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, NR^{1B}SO₂R^{1A}, —NR^{1B}C(O)R^{D}, —NR^{1B}C(O)OR^{D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof. R^{1A}, R^{1B}, R^{1C}, and R^{1D} are independently hydrogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{1B} and R^{1C} substituents bonded to the same nitrogen atom may optionally be joined to form a substituted or unsubstituted heterocycloalkyl or substituted or unsubstituted heteroaryl. The symbol “ [symbol] ” indicates the point of attachment between the peptidase and the inhibitor. The symbol n1 is an integer from 0 to 4. The symbols m1 and v1 are independently 1 or 2. The symbol z1 is an integer from 1 to 4. The symbol z2 is 0 or 1.

In embodiments, W comprises at least one amino acid selected from 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

In embodiments, W comprises glycine and at least one amino acid selected from: 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

In embodiments, the compound is:

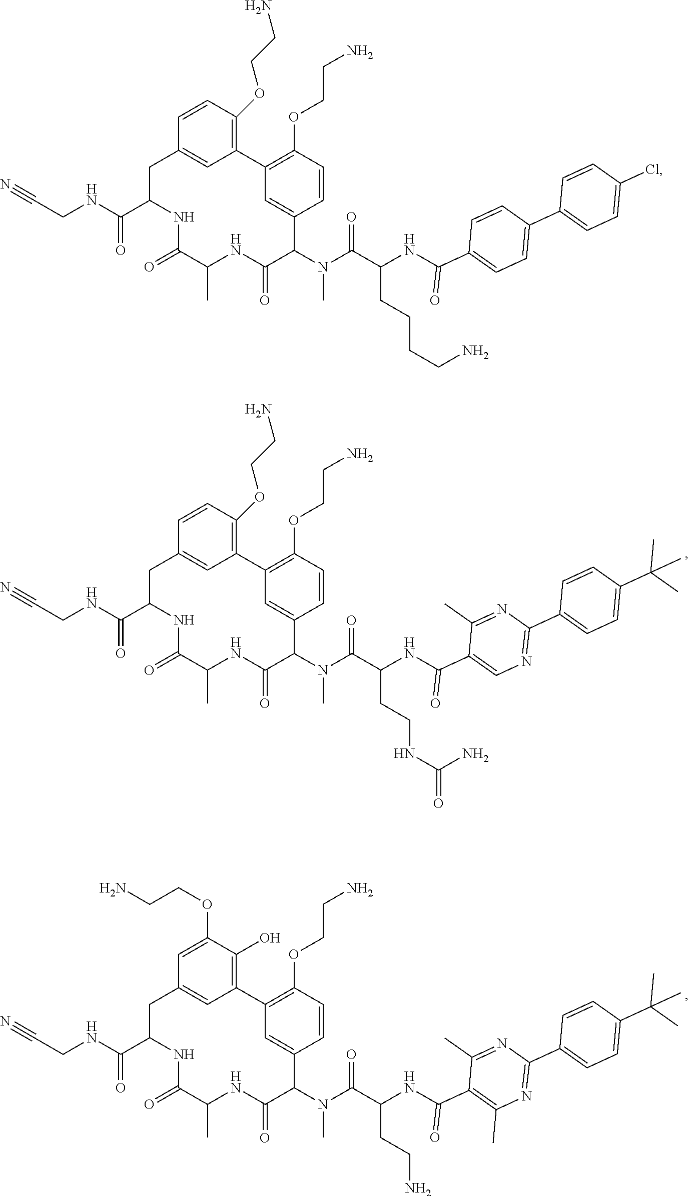 [SMILES: CC1NC(=O)C(N(C)C(=O)C(CCCCN)NC(=O)c2ccc(-c3ccc(Cl)cc3)cc2)c2ccc(OCCN)c(c2)-c2cc(ccc2OCCN)CC(C(=O)NCC#N)NC1=O ; Cc1nc(-c2ccc(C(C)(C)C)cc2)ncc1C(=O)NC(CCNC(N)=O)C(=O)N(C)C1C(=O)NC(C)C(=O)NC(C(=O)NCC#N)Cc2ccc(OCCN)c(c2)-c2cc1ccc2OCCN ; Cc1nc(-c2ccc(C(C)(C)C)cc2)nc(C)c1C(=O)NC(CCN)C(=O)N(C)C1C(=O)NC(C)C(=O)NC(C(=O)NCC#N)Cc2cc(OCCN)c(O)c(c2)-c2cc1ccc2OCCN]

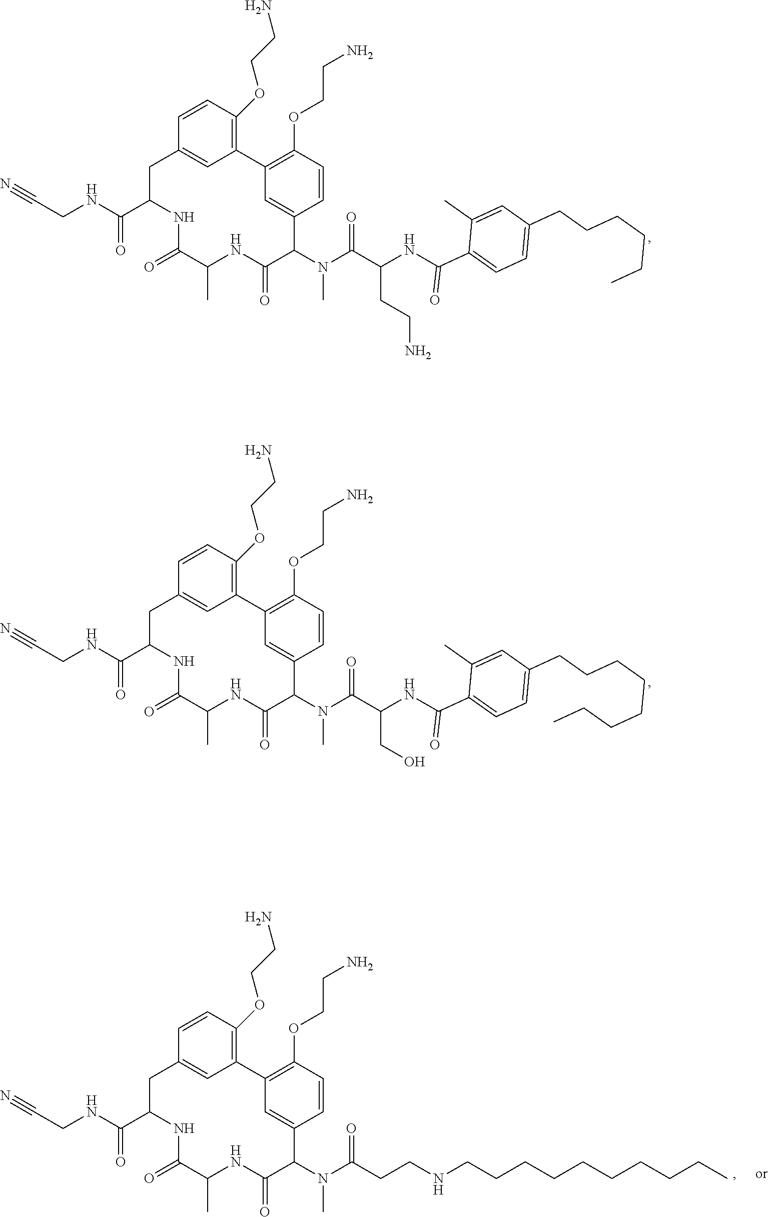 [SMILES: CCCCCCc1ccc(C(=O)NC(CCN)C(=O)N(C)C2C(=O)NC(C)C(=O)NC(C(=O)NCC#N)Cc3ccc(OCCN)c(c3)-c3cc2ccc3OCCN)c(C)c1 ; CCCCCCCCc1ccc(C(=O)NC(CO)C(=O)N(C)C2C(=O)NC(C)C(=O)NC(C(=O)NCC#N)Cc3ccc(OCCN)c(c3)-c3cc2ccc3OCCN)c(C)c1 ; CCCCCCCCCCNCCC(=O)N(C)C1C(=O)NC(C)C(=O)NC(C(=O)NCC#N)Cc2ccc(OCCN)c(c2)-c2cc1ccc2OCCN]

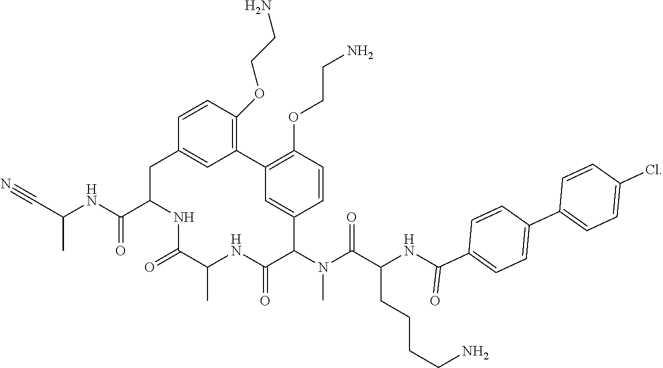 [SMILES: CC(C#N)NC(=O)C1Cc2ccc(OCCN)c(c2)-c2cc(ccc2OCCN)C(N(C)C(=O)C(CCCCN)NC(=O)c2ccc(-c3ccc(Cl)cc3)cc2)C(=O)NC(C)C(=O)N1]

In embodiments, the compound has structural Formula (IV-B):

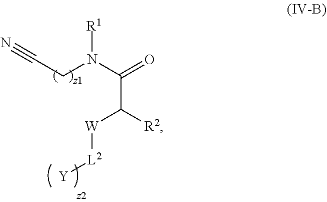

or a pharmaceutically acceptable salt thereof.

z1, z2, R¹, W, Y, and L² are as described herein, including embodiments. R² is independently hydrogen, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound has structural Formula (IV-C):

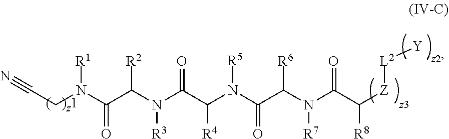

or a pharmaceutically acceptable salt thereof.

z1, z2, R¹, R², Y, and L² are as described herein, including embodiments.

The symbol z3 is an integer from 0 to 20. R⁴ and R⁶ are independently hydrogen,

halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² and R⁶; or R² and R⁸ may optionally be joined to form a substituted or unsubstituted heterocycloalkyl. R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl-C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl-C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H) [optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl. Z is independently a substituted or unsubstituted natural amino acid or a substituted or unsubstituted unnatural amino acid. In embodiments, R² and a Z amino acid; R⁴ and a Z amino acid; R⁶ and a Z amino acid; or two Z amino acids may optionally be joined by a linking moiety, L³, to form a substituted or unsubstituted heterocycloalkyl. L³ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R^{8A} is independently hydrogen or —(C₁-C₆)alkyl. R^{8B} and R^{8C} are independently hydrogen or optionally substituted —(C₁-C₆)alkyl or R^{8B} and R^{8C} and the nitrogen atom to which they are attached optionally form a heterocycloalkyl ring. R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R′⁴)₂, —C(O)N(R′⁴)₂, or —SO₂N(R¹⁴)₂. In embodiments, R^{8D} and R^{8E} and the nitrogen atom to which they are attached optionally form a heterocycloalkyl ring. R¹⁴ is independently hydrogen or —(C₁-C₆)alkyl. In embodiments, two R¹⁴ groups and the nitrogen atom to which they are attached optionally form a heterocycloalkyl ring.

In embodiments, R² and R⁶ are joined to form a substituted or unsubstituted heterocycloalkyl.

In embodiments, z3 is 0.

In embodiments, Z comprises at least one amino acid selected from 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

In embodiments, Z comprises glycine and at least one amino acid selected from: 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid. In embodiments, Z comprises glycine.

In embodiments, the compound has structural Formula (V):

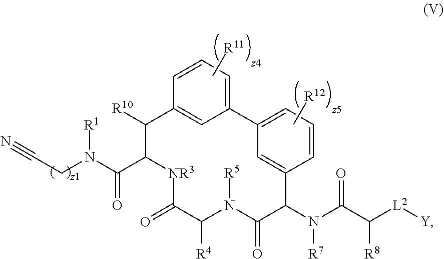

or a pharmaceutically acceptable salt thereof.

z1, R¹, R³, R⁴, R⁵, R⁷, R⁸, Y, and L² are as described herein, including embodiments. The symbols z4 and z5 are independently an integer from 0 to 4.

R¹⁰ is hydrogen,

halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹¹ is hydrogen,
halogen, —CCl₃, —CBr₃, —CF₃, —CI₃, —CN, —OR^{2A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹² is hydrogen,
halogen, —CCl₃, —CBr₃, —CF₃, —CI₃, —CN, —OR^{3A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound has structural Formula (V-A):

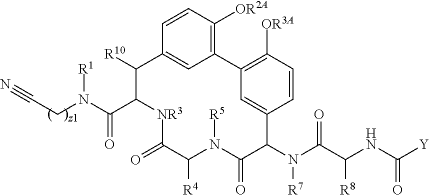

(V-A), or a pharmaceutically acceptable salt thereof,

z1, R¹, R³, R⁴, R⁵, R⁷, R⁸, R¹⁰, R^{2A}, R^{3A}, and Y are as described herein, including embodiments.

In embodiments, R^{2A} and R^{3A} are independently hydrogen, substituted or unsubstituted alkyl, or substituted or unsubstituted heteroalkyl.

In embodiments, Y is substituted or unsubstituted aryl or substituted or unsubstituted heteroaryl.

In embodiments, R^{2A} and R^{3A} are independently substituted or unsubstituted heteroalkyl.

In embodiments, the compound has structural Formula (V-B):

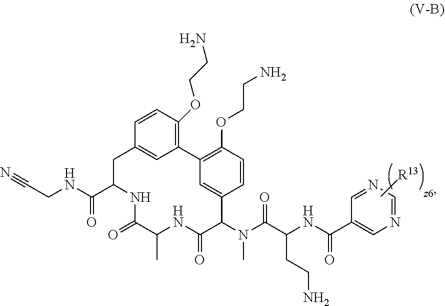

or a pharmaceutically acceptable salt thereof.

z6 and R¹³ are as described herein, including embodiments.

In embodiments, R¹³ is independently substituted or unsubstituted alkyl, substituted or unsubstituted aryl, or substituted or unsubstituted heteroaryl.

In embodiments, the compound has structural Formula (V-C):

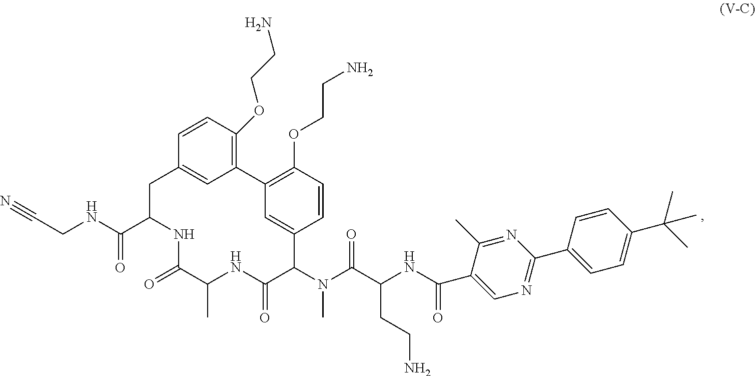 [SMILES: Cc1nc(-c2ccc(C(C)(C)C)cc2)ncc1C(=O)NC(CCN)C(=O)N(C)C1C(=O)NC(C)C(=O)NC(C(=O)NCC#N)Cc2ccc(OCCN)c(c2)-c2cc1ccc2OCCN]

or a pharmaceutically acceptable salt thereof.

In embodiments, the compound has structural Formula (V-D):

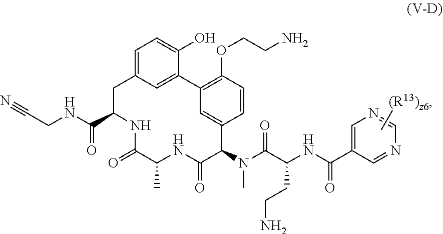

or a pharmaceutically acceptable salt thereof.

z6 and R¹³ are as described herein, including embodiments.

In embodiments, the bacterial peptidase is a signal peptidase (SPase). In embodiments, the SPase is a bacterial type I SPase.

In embodiments, the compound forms an irreversible bond with the bacterial peptidase. In embodiments, the bond is a covalent bond.

In embodiments, the peptidase comprises region B, region C, region C′, and region D. B comprises amino acid sequence PSXSMXPTLX (SEQ ID NO: 1). C comprises amino acid sequence DXIXVXKXX (SEQ ID NO: 2). C′ comprises amino acid sequence RGDXXVFXXP (SEQ ID NO: 3).

D comprises amino acid sequence Y/F, I/V, KRXXGXXGD (SEQ ID NO: 4). X is any natural amino acid residue or any unnatural amino acid residue.

In embodiments, the bacterial peptidase and the compound bond at region D of the peptidase.

In embodiments, the compound forms a bond with a lysine residue of the bacterial peptidase. In embodiments, the bond is formed at the side chain of the lysine residue.

In embodiments, the bacterial peptidase is LepB. In embodiments, the lysine residue is Lys¹⁴⁶

In embodiments, the bacterial peptidase is selected from E. coli, L. monocytogenes, M. leprae, M. tuberculosis, M. ulcerans, M. pneumoniae, K. pneumoniae, K. pneumoniae, E. aerogenes, C. werkmanii, S. marcescens, S. marcescens, A. baumannii, N. gonorrhoeae, and N. meningitides.

In an aspect provided herein, is a method of inhibiting signal protein secretion of a bacterial cell, comprising contacting the cell with a compound of structural Formula (IV):

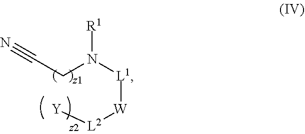

or a pharmaceutically acceptable salt thereof.

z1, z2, R¹, W, Y, L¹, and L² are as described herein, including embodiments.

In an aspect provided herein, is a method of treating a bacterial infection, comprising administering to a subject in need thereof a therapeutically effective amount of a compound of structural Formula (IV):

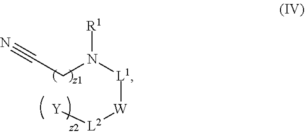

or a pharmaceutically acceptable salt thereof.

z1, z2, X¹, R¹, W, Y, L¹, and L² are as described herein, including embodiments.

In an aspect provided herein, is a method of treating a bacterial infection, comprising administering to a subject in need thereof a therapeutically effective amount of a compound of structural Formula (IV-C):

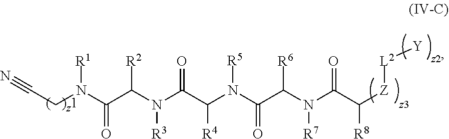

or a pharmaceutically acceptable salt thereof.

z1, z2, z3, R¹, R², R³, R⁴, R⁵, R⁶, R⁷, R⁸, Z, Y, and L² are as described herein, including embodiments.

In an aspect provided herein, is a method of treating a bacterial infection, comprising administering to a subject in need thereof a therapeutically effective amount of a compound of structural Formula (V):

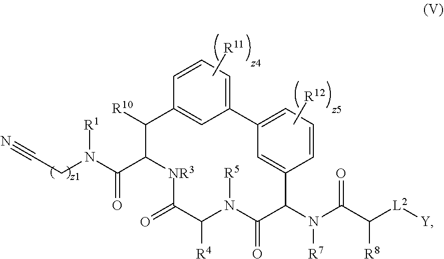

or a pharmaceutically acceptable salt thereof.

z1, z5, z6, R¹, R³, R⁴, R⁵, R⁶, R⁷, R⁸, R¹⁰, R¹¹, R¹², Y, and L² are as described herein, including embodiments.

In an aspect provided herein, is a method of treating a bacterial infection, comprising administering to a subject in need thereof a therapeutically effective amount of a compound of structural Formula (V-A):

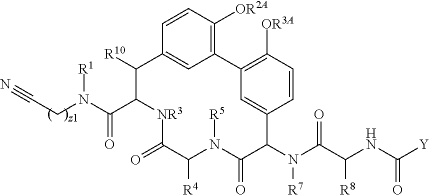

(V-A), or a pharmaceutically acceptable salt thereof,

z1, R¹, R², R³, R⁴, R⁵, R⁶, R⁷, R⁸, R¹⁰, R^{2A}, R^{3A}, and Y are as described herein, including embodiments.

In an aspect provided herein, is a method of treating a bacterial infection, comprising administering to a subject in need thereof a therapeutically effective amount of a compound of structural Formula (V-B):

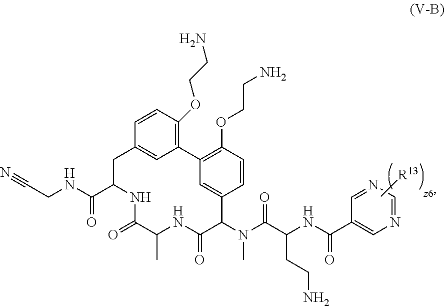

or a pharmaceutically acceptable salt thereof.

z6 and R¹³ are as described herein, including embodiments.

In an aspect provided herein, is a method of treating a bacterial infection, comprising administering to a subject in need thereof a therapeutically effective amount of a compound of structural Formula (V-C):

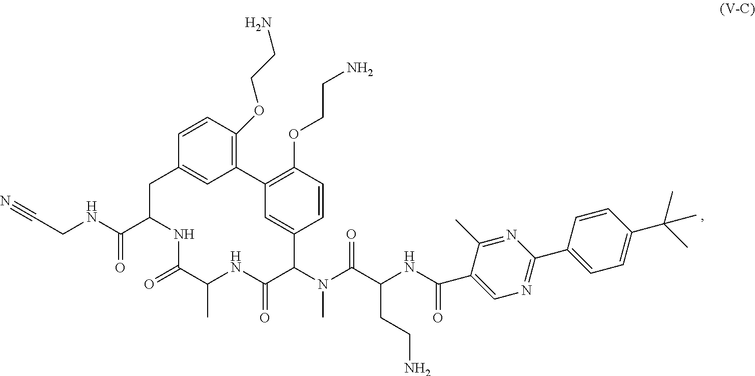 [SMILES: Cc1nc(-c2ccc(C(C)(C)C)cc2)ncc1C(=O)NC(CCN)C(=O)N(C)C1C(=O)NC(C)C(=O)NC(C(=O)NCC#N)Cc2ccc(OCCN)c(c2)-c2cc1ccc2OCCN]

or a pharmaceutically acceptable salt thereof.

In an aspect provided herein, is method of treating a bacterial infection, comprising administering to a subject in need thereof a therapeutically effective amount of a compound of structural Formula (V-D):

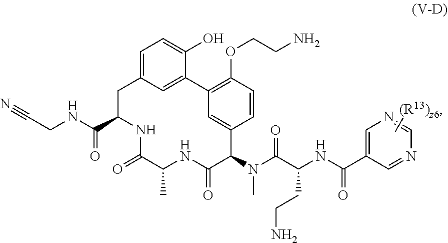

or a pharmaceutically acceptable salt thereof.

z6 and R¹³ are as described herein, including embodiments.

In embodiments, the bacterial infection is caused by E. coli, L. monocytogenes, M. leprae, M. tuberculosis, M. ulcerans, M. pneumoniae, K. pneumoniae, K. pneumoniae, E. aerogenes, C. werkmanii, S. marcescens, S. marcescens, A. baumannii, N. gonorrhoeae, or N. meningitides.

In embodiments, the bacterial infection is caused by methicillin-resistant (MRSA) bacteria, vancomycin-intermediate (VISA) bacteria, vancomycin-resistant Staphylococcus aureus (VRSA), multidrug-resistant (MDR) bacteria, pandrug-resistant (PDR) Gram-negative bacteria, or extensively drug-resistant (XDR) bacteria.

In embodiments, the PDR Gram-negative bacteria is Pseudomonas aeruginosa, Acinetobacter baumannii, or Klebsiella pneumoniae.

In embodiments, the PDR and XDR bacteria are independently Mycobacterium tuberculosis.

In embodiments, the bacterial infection is caused by a Gram-negative bacteria.

In embodiments, the bacterial infection is caused an indwelling device or a prosthetic device.

In embodiments, the bacterial infection is caused by a biofilm-associated bacteria.

In embodiments, the method of treating the bacterial infection in a subject in need thereof further comprises administering to the subject an antibiotic in combination with the compound of structural Formulae (IV), (IV-C), (V), (V-A), (V-B), (V-C), or (V-D), including embodiments, or a pharmaceutically acceptable salt thereof.

In embodiments, the antibiotic is an aminoglycoside, a fluoroquinolone, a carbapenem, a tetracycline, or an arylomycin. In embodiments, the antibiotic is ceftazidime, avibactam, levofloxacin, meropenem, colistin, or tigecycline.

In embodiments, the method of treating the bacterial infection in a subject in need thereof, comprising administering to the subject a pharmaceutical composition, comprising a compound of structural Formulae (IV), (IV-C), (V), (V-A), (V-B), (V-C), or (V-D), including embodiments, or a pharmaceutically acceptable salt thereof, and a pharmaceutically acceptable excipient.

In an aspect provided herein, is a method of inhibiting a peptide, comprising contacting a peptide comprising a serine-lysine catalytic dyad or a serine-serine lysine catalytic triad with a compound of structural Formula (IV):

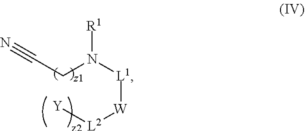

or a pharmaceutically acceptable salt thereof, wherein the compound of Formula (IV) forms a bond to an amino group of the lysine in the dyad or triad.

z1, z2, X¹, R¹, W, Y, L¹, and L² are as described herein, including embodiments.

In an aspect provided herein, is a method of inhibiting a peptide, comprising contacting a peptide comprising a serine-lysine catalytic dyad or a serine-serine lysine catalytic triad with a compound of structural Formula (IV-C):

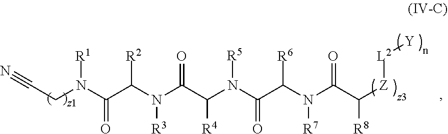

or a pharmaceutically acceptable salt thereof, wherein the compound of Formula (IV-C) forms a bond to an amino group of the lysine in the dyad or triad.

z1, z2, z3, R¹, R², R³, R⁴, R⁵, R⁶, R⁷, R⁸, Z, Y, and L² are as described herein, including embodiments.

In an aspect provided herein, is a method of inhibiting a peptide, comprising contacting a peptide comprising a serine-lysine catalytic dyad or a serine-serine lysine catalytic triad with a compound of structural Formula (V):

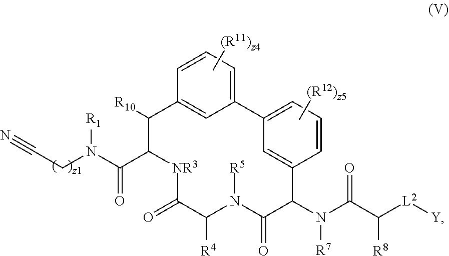

or a pharmaceutically acceptable salt thereof, wherein the compound of Formula (V) forms a bond to an amino group of the lysine in the dyad or triad.

z1, z5, z6, R¹, R³, R⁴, R⁵, R⁶, R⁷, R⁸, R¹⁰, R¹¹, R¹², Y, and L² are as described herein, including embodiments.

In an aspect provided herein, is a method of inhibiting a peptide, comprising contacting a peptide comprising a serine-lysine catalytic dyad or a serine-serine lysine catalytic triad with a compound of structural Formula (V-A):

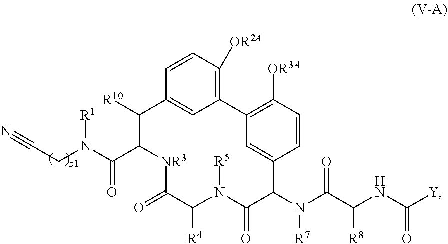

or a pharmaceutically acceptable salt thereof, wherein the compound of Formula (V-A) forms a bond to an amino group of the lysine in the dyad or triad,

z1, R¹, R², R³, R⁴, R⁵, R⁶, R⁷, R⁸, R¹⁰, R^{2A}, R^{3A}, and Y are as described herein, including embodiments.

In an aspect provided herein, is a method of inhibiting a peptide, comprising contacting a peptide comprising a serine-lysine catalytic dyad or a serine-serine lysine catalytic triad with a compound of structural Formula (V-B):

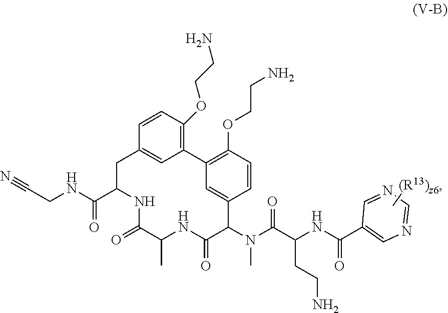

or a pharmaceutically acceptable salt thereof, wherein the compound of Formula (V-B) forms a bond to an amino group of the lysine in the dyad or triad.

z6 and R¹³ are as described herein, including embodiments.

In an aspect provided herein, is a method of inhibiting a peptide, comprising contacting a peptide comprising a serine-lysine catalytic dyad or a serine-serine lysine catalytic triad with a compound of structural Formula (V-C):

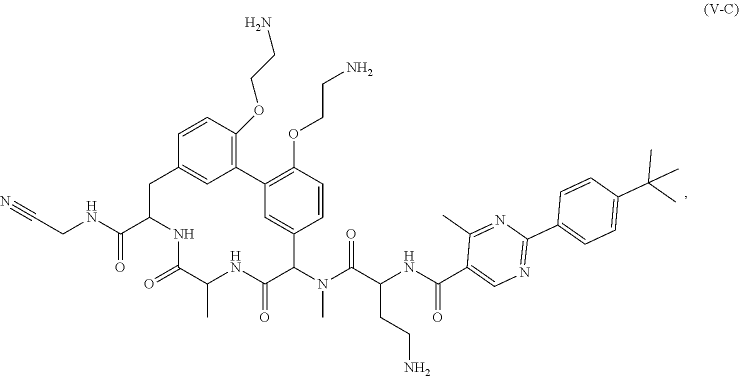 [SMILES: Cc1nc(-c2ccc(C(C)(C)C)cc2)ncc1C(=O)NC(CCN)C(=O)N(C)C1C(=O)NC(C)C(=O)NC(C(=O)NCC#N)Cc2ccc(OCCN)c(c2)-c2cc1ccc2OCCN ; ","]

or a pharmaceutically acceptable salt thereof, wherein the compound of Formula (V-C) forms a bond to an amino group of the lysine in the dyad or triad.

In an aspect provided herein, is a method of inhibiting a peptide, comprising contacting a peptide comprising a serine-lysine catalytic dyad or a serine-serine lysine catalytic triad with a compound of structural Formula (V-D):

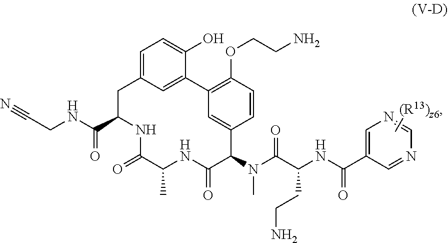

or a pharmaceutically acceptable salt thereof, wherein the compound of Formula (V-D) forms a bond to an amino group of the lysine in the dyad or triad.

z6 and R¹³ are as described herein, including embodiments.

In embodiments, X¹ is ═O. In embodiments, X¹ is ═NH.

In embodiments, n1 is 0. In embodiments, n1 is 1. In embodiments, n1 is 2. In embodiments, n1 is 3. In embodiments, n1 is 4.

In embodiments, m1 is 1. In embodiments, m1 is 2. In embodiments, v1 is 1. In embodiments, v1 is 2.

In embodiments, z is 0. In embodiments, z is 1. In embodiments, z is 2. In embodiments, z is 3. In embodiments, z1 is 4.

In embodiments, z2 is 0. In embodiments, z2 is 1.

In embodiments, z3 is 0. In embodiments, z3 is 1. In embodiments, z3 is 2. In embodiments, z3 is 3. In embodiments, z3 is 4. In embodiments, z3 is 5. In embodiments, z3 is 6. In embodiments, z3 is 7. In embodiments, z3 is 8. In embodiments, z3 is 9. In embodiments, z3 is 10. In embodiments, z3 is 11. In embodiments, z3 is 12. In embodiments, z3 is 13. In embodiments, z3 is 14. In embodiments, z3 is 15. In embodiments, z3 is 16. In embodiments, z3 is 17. In embodiments, z3 is 18. In embodiments, z3 is 19. In embodiments, z3 is 20.

In embodiments, z4 is 0. In embodiments, z4 is 1. In embodiments, z4 is 2. In embodiments, z4 is 3. In embodiments, z4 is 4.

In embodiments, z5 is 0. In embodiments, z5 is 1. In embodiments, z5 is 2. In embodiments, z5 is 3. In embodiments, z5 is 4.

In embodiments, z6 is 0. In embodiments, z6 is 1. In embodiments, z6 is 2. In embodiments, z6 is 3.

### IV. Examples

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

### Example 1. Arylomycin can be Optimized into Inhibitors of Gram-Negative ESKAPE Pathogens

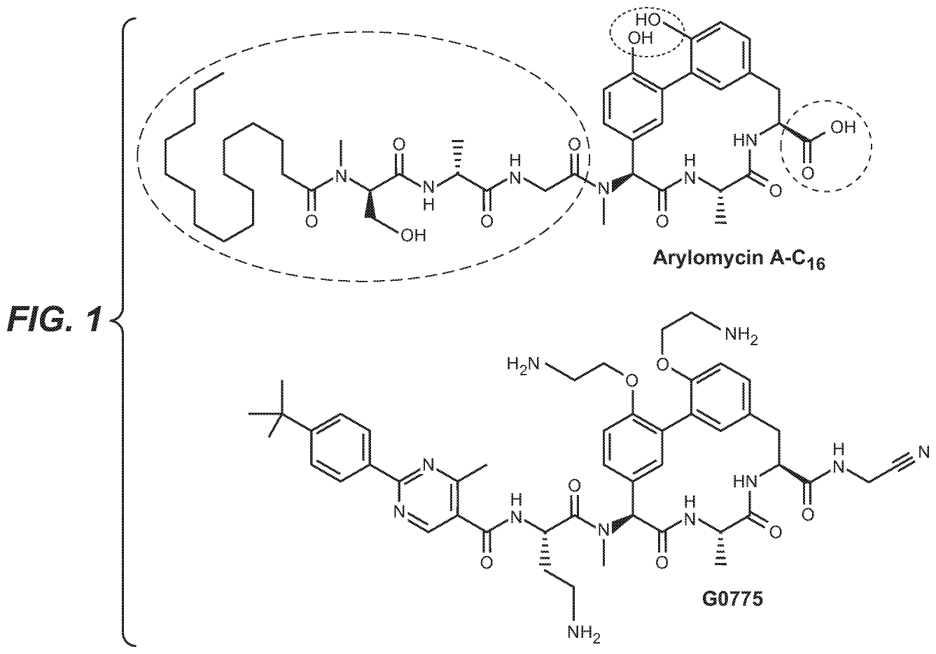

With the goal of modifying the natural product arylomycin into a broadly active Gram-negative antibiotic, three discrete positions in the scaffold were examined as potential areas for modification. The molecule consists of a macrocyclic core region that serves as a platform displaying an N-terminal lipopeptide tail, two macrocycle bisphenols, and a C-terminal carboxylate (FIG. 1). Because crystal structures of the Gram-negative type I signal peptidase (LepB) in complex with arylomycin indicate that the macrocyclic core preorganizes the peptide backbone and occupies a conserved region of the substrate binding pocket, this region was left unchanged and chemistry efforts were focused on the three peripheral positions. Naturally occurring variants of the arylomycin N-terminal lipopeptide tail are known to modulate the spectrum of Gram-positive activity, so variants that improve Gram-negative activity were first searched for. The linear D-N-Me-Ser-D-Ala-Gly tripeptide in arylomycin was shortened to a single diaminobutyric acid and replaced the natural aliphatic tail with 2-(4-(tert-butyl)phenyl)-4-methylpyrimidine-5-carboxylic acid. Secondly, inspired by modifications to the macrocyclic phenols found in natural arylomycin analogues and a previous report that appending a single ethyl amine to the phenols affords modest activity gains, simultaneous substitution off of both phenols was explored. Finally, electrophilic warheads were appended at the C-terminal carboxylate in an attempt to covalently bind the catalytic serine, and 2-aminoacetonitrile was identified as the optimal solution (FIG. 1). The final molecule (G0775) is >500-fold more potent than arylomycin A-C₁₆ against the ESKAPE pathogens E. coli and K. pneumoniae as well as related pathogens from the same family. Moreover, the activity of G0775 extends to the notoriously difficult to treat non-fermenting Gram-negative bacteria Pseudomonas aeruginosa and Acinetobacter baumannii (Table 1). Thus, G0775 validates the hypothesis that chemical modifications can be used to expand the spectrum of the arylomycins to include ESKAPE pathogens and represents a novel and exciting molecule that could address the need for new Gram-negative antibiotics.

TABLE 1
MIC values for arylomycin A-C₁₆ and G0775
against pathogenic Gram-negative bacteria

MIC (μg/ml)

| Bacterial Species | Arylomycin A-C₁₆ | G0775 |
|---|---|---|
| E. coli ATCC 25922 | >64 | 0.125 |
| K. pneumoniae ATCC 43816 | >64 | 0.125 |
| E. cloacae ATCC 13407 | >64 | 0.25 |
| E. aerogenes ATCC 13408 | >64 | 0.125 |
| C. werkmanii ATCC 51114 | >64 | 0.125 |
| S. marcescens CDC4385-74 | >64 | 0.125 |
| P. aeruginosa ATCC 27853 | >64 | 4 |
| A. baumannii ATCC 17978 | >64 | 1 |

### Example 2. G0775 is Active Against MDR Bacteria

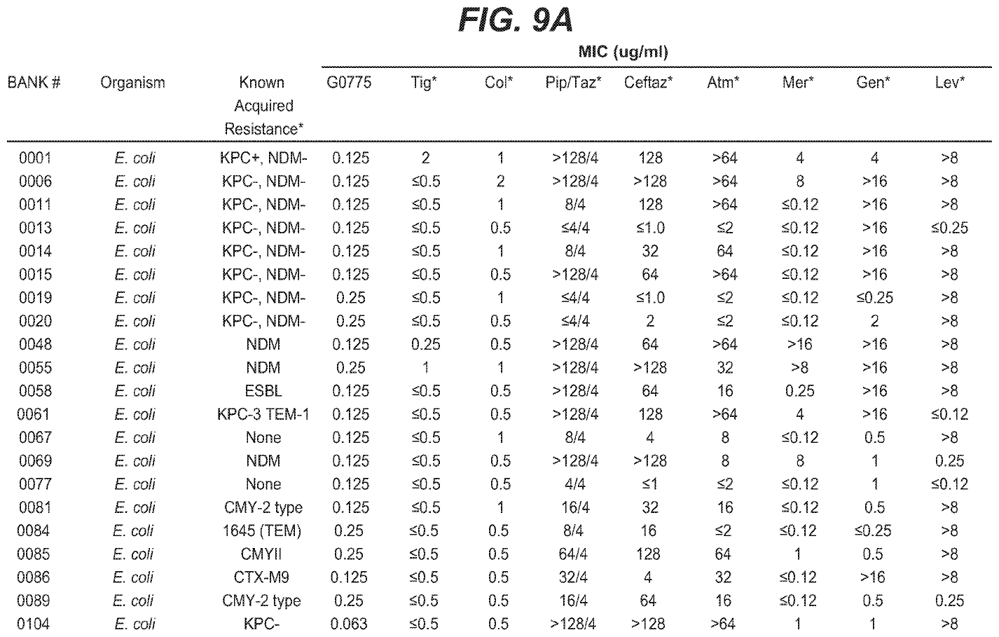

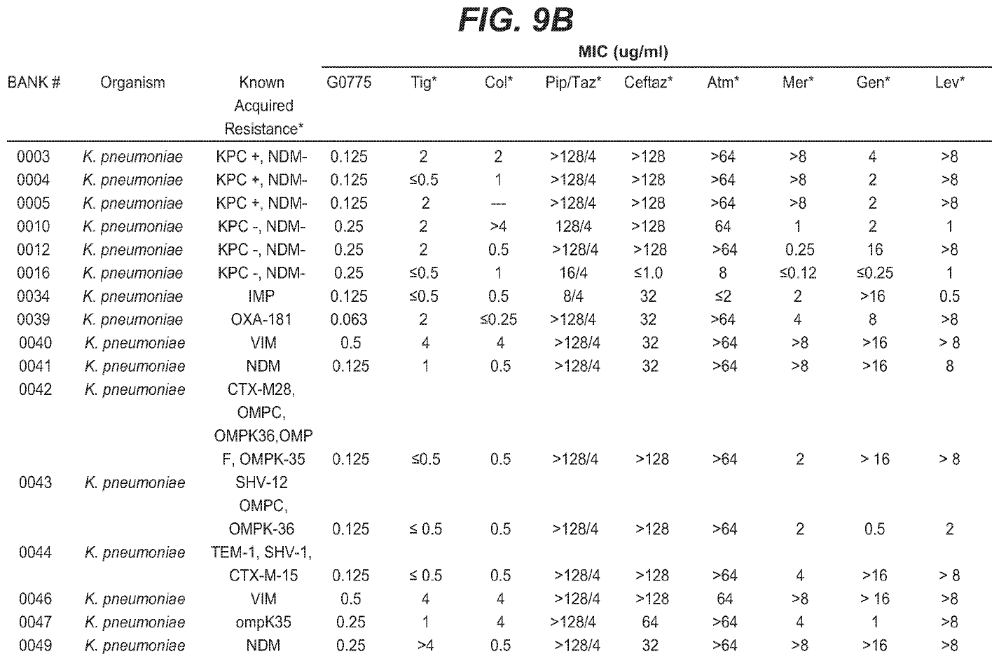

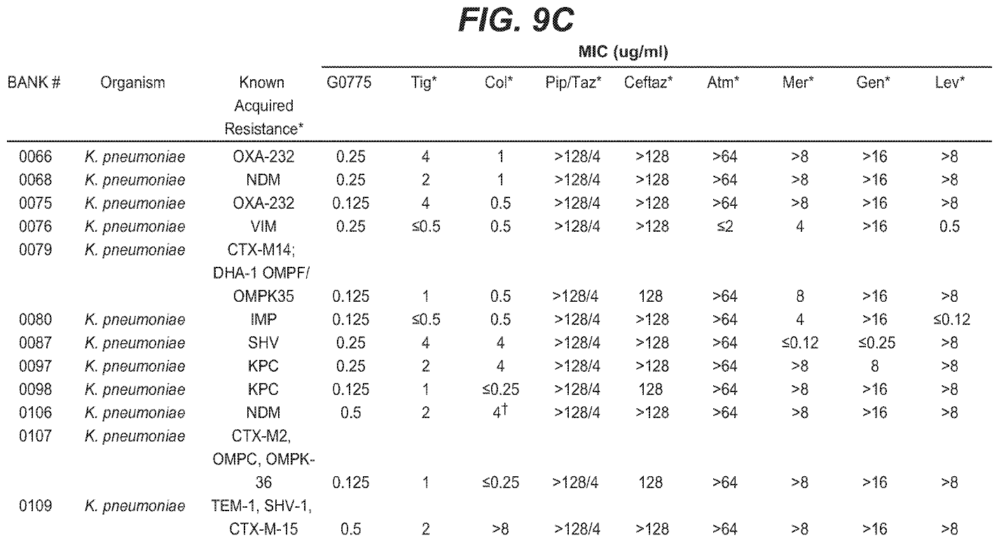

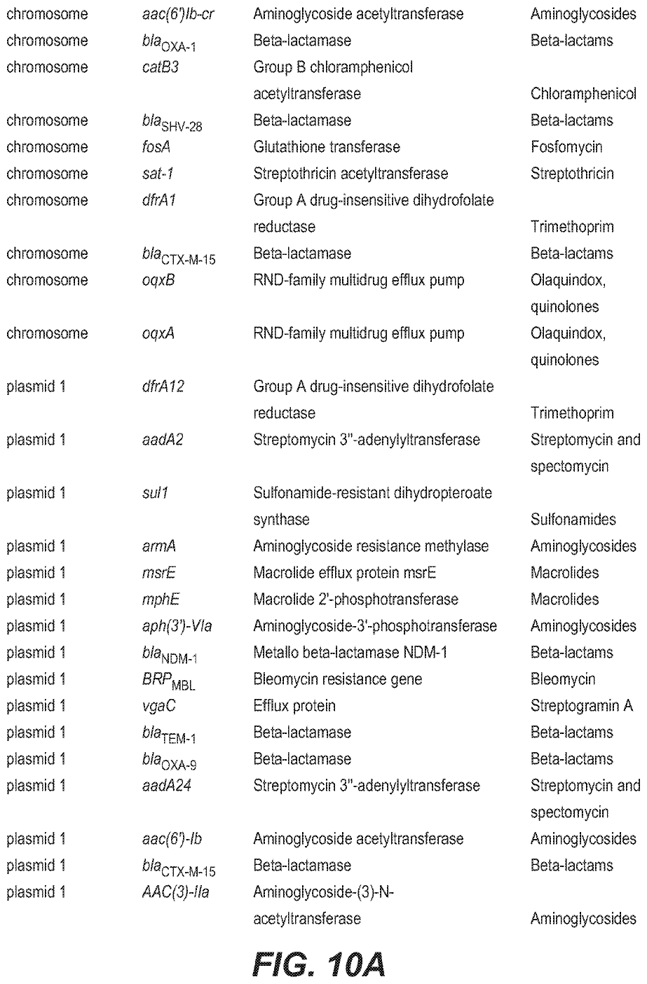

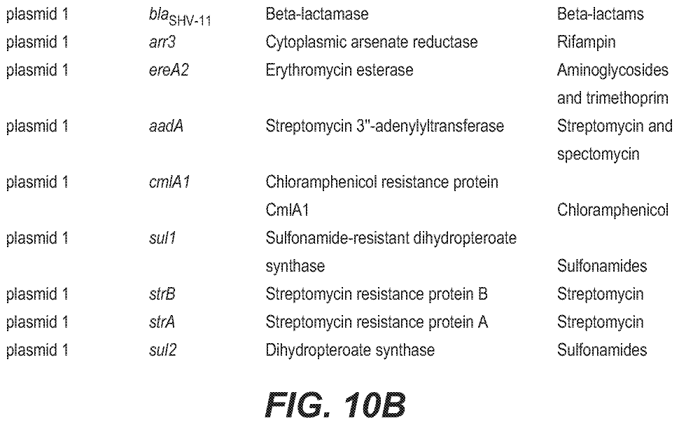

Having demonstrated potency against common laboratory strains of Gram-negative ESKAPE pathogens, whether or not G0775 circumvents the plethora of resistance mechanisms that undermine the effectiveness of currently available therapies was explored. First, the Minimum Inhibitory Concentration (MIC) of G0775 against a panel of 49 multidrug resistant clinical isolates of E. coli and K. pneumoniae that the Centers for Disease Control and Prevention provides as a challenge set to evaluate potential novel antibiotic agents was determined. The panel is highly enriched for MDR pathogens, with more than half exhibiting resistance to at least 5 of the 8 antibiotics examined. In contrast to the approved agents, G0775 maintains its potent activity against all 49 isolates (FIGS. 9A-9C). To better assess the ability of G0775 to circumvent specific resistance mechanisms, a single MDR isolate, CDC 0106, was focused on. Whole genome sequencing (WGS) of CDC 0106 revealed at least 10 chromosomally encoded and 25 plasmid encoded genes associated with resistance to 13 classes of antibiotics. Chromosomal mutations associated with high level quinolone resistance and polymyxin resistance were also identified (FIGS. 10A-10B). Importantly, the elements encoded by CDC 0106 confer resistance to nearly all available antibiotic classes (Table 2).

TABLE 2
MIC values for G0775 and antibiotics from diverse
classes against MDR K. pneumoniae CDC 0106.

| Antibiotic Class | Antibiotic | MIC (μg/mL) |
|---|---|---|
| Novel BL/BLI | Ceftazidime/avibactam | >64 |
| Aminoglycoside | Gentamicin | >64 |
| Fluoroquinolone | Levofloxacin | >64 |
| Carbapenem | Meropenem | >64 |
| Polymyxin | Colistin | 16 |
| Tetracycline | Tigecycline | 2 |
| Arylomycin | G0775 | 0.5 |

Moreover the presence of genes encoding the NDM-1 metallo beta-lactamase and the ArmA ribosomal methylase are expected to confer resistance even to novel therapies currently in late stage clinical trials such as Carbavance and Plazomicin, respectively, which are novel variants of known antibiotic classes. G0775 remains highly active against CDC 106, confirming that this novel class is not affected by this formidable repertoire of resistance elements.

### Example 3. G0775 Acts Via LepB Inhibition and has a Low Frequency of Resistance

It was next set out to confirm that the improved antibacterial activity of G0775 was achieved through inhibition of LepB and not via off target or secondary mechanisms. G0775's cellular potency as a function of target expression levels was first measured and it was found that under- and over-expression of LepB in E. coli K12 MG1655 leads to a respective 16-fold and 8-fold decrease and increase in MIC values (Table 3).

TABLE 3
G0775 MIC values measured against E. coli K-12 MG1655 and isogenic matched
strains with the indicated genetic or pharmacological manipulation.

| Strain | Phenotype | G0775 MIC (μg/ml) |
|---|---|---|
| MG1655 WT | WT | 0.125 |
| MG 1655 - LepB Low | Reduced LepB expression level | 0.008 |
| MG 1655 - LepB High | Increase LepB expression level | 1 |
| MG 1655 ΔacrB | Efflux null | 0.125 |
| MG 1655 ΔtolC | Efflux null | 0.125 |
| MG 1655 ΔompC | Porin deficient | 0.125 |
| MG 1655 ΔompF | Porin deficient | 0.125 |
| MG 1655 ΔompC, ΔompF | Doubly porin deficient | 0.125 |
| MG 1655 LptD4213 (imp) | Outer-membrane permeable | 0.004 |
| MG1655 WT + 4 mM EDTA | Outer-membrane permeable | 0.004 |
| MG1655 WT + 50% serum | Plasma protein binding | 0.125 |
| MG1655 WT + 1% | Lung inactivation | 0.125 |
| surfactant | | |

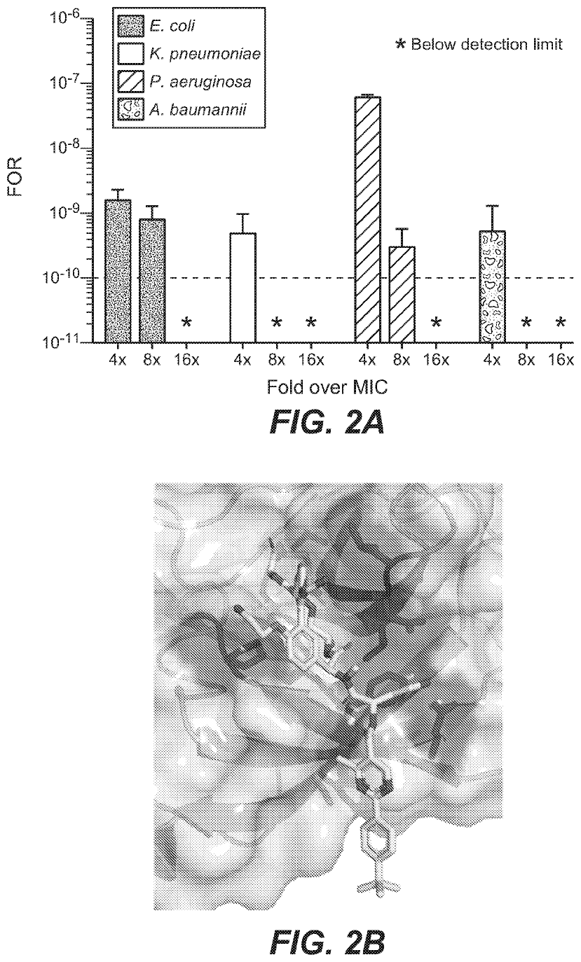

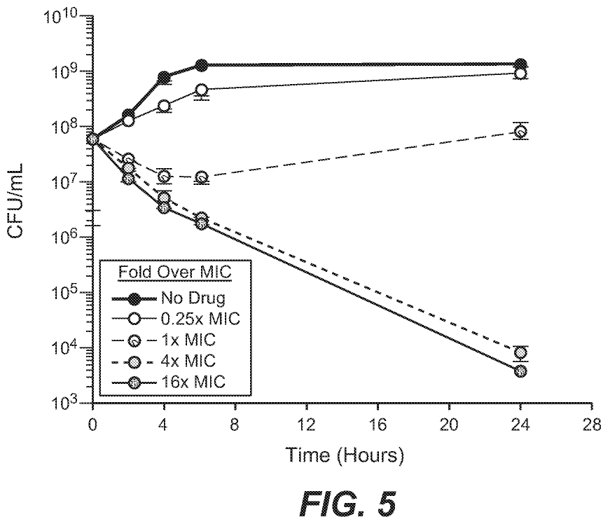

Next, the rate at which G0775 kills bacteria was explored. Genetic depletion of lepB in E. coli leads to time-dependent cell death across a 24 hour period and likewise, G0775 effects a time-dependent killing at a similar rate, ultimately reducing the number of viable bacteria by over 6 orders of magnitude (FIG. 5). The target titration and time-kill data are consistent with G0775 acting via LepB inhibition, and to unequivocally confirm this, the ability of diverse Gram-negative ESKAPE pathogens to spontaneously evolve resistance to G0775 was assessed. As expected for an inhibitor targeting a single gene product, resistant mutants were isolated at a moderate frequency in the presence of low G0775 concentrations, 4-fold the MIC. Importantly, increasing the concentration of G0775 to 8-fold the MIC reduced the frequency of resistance (FOR) below the limit of detection for E. coli, K. pneumoniae and A. baumannii and a further 2-fold increase reduced the P. aeruginosa FOR to <10⁻¹⁰ (FIG. 2A). Targeted and whole genome sequencing of resistant E. coli mutants revealed that all but one of the resistance-conferring mutations were found within the substrate/inhibitor-binding groove of LepB, confirming that the whole cell activity of G0775 is on target. Encouragingly, these mutations reduce susceptibility to G0775 by only 4- to 16-fold (Table 4), consistent with the dramatic decrease in resistance frequencies in the presence of higher drug concentrations.

TABLE 4
G0775 MIC values measured against E. coli ATCC 25922 point mutants generated
spontaneously in response to plating on 4x MIC concentration of G0775.

| | | | Fold MIC |
|---|---|---|---|
| Background | Mutation | LepB residue, E. coli numbering | Increase |
| E. coli ATCC 25922 | E83K | 83 | 16 |
| E. coli ATCC 25922 | F85S | 85 | 8 |
| E. coli ATCC 25922 | P88L | 88 | 16 |
| E. coli ATCC 25922 | P88Q | 88 | 4 |
| E. coli ATCC 25922 | P88R | 88 | 16 |
| E. coli ATCC 25922 | P88T | 88 | 4 |
| E. coli ATCC 25922 | V133F | 133 | 16 |
| E. coli ATCC 25922 | L142R | 142 | 8 |
| E. coli ATCC 25922 | D143Y | 143 | 8 |
| E. coli ATCC 25922 | V149(VVGLP) | 149 | 128 |
| E. coli ATCC 25922 | A297V | 297 | 4 |
| E. coli ATCC 25922 | AcrB (N282Y) | — | 4 |

The only off-target mutation discovered was a single missense mutation (N282Y) in the efflux pump encoding gene acrB, which may increase the affinity of this efflux system for G0775. Interestingly, deletion of AcrB or TolC efflux pumps in the wild type MG1655 background had no impact on the potency of G0775, supporting the notion that AcrB N282Y is a gain of function mutation and that G0775 is not ordinarily a substrate of these efflux systems in E. coli (Table 3). In total, these results demonstrate that G0775 kills Gram-negative bacteria via a novel mechanism, and that spontaneous resistance to G0775 occurs primarily via target mutants that provide only modest decreases in susceptibility.

### Example 4. G0775 Binds to LepB with an Unprecedented MOI

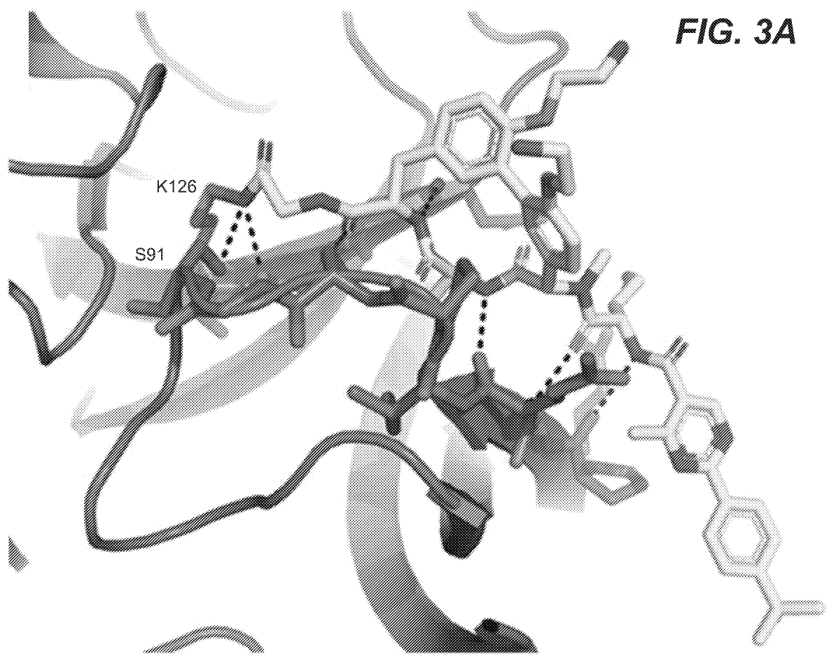

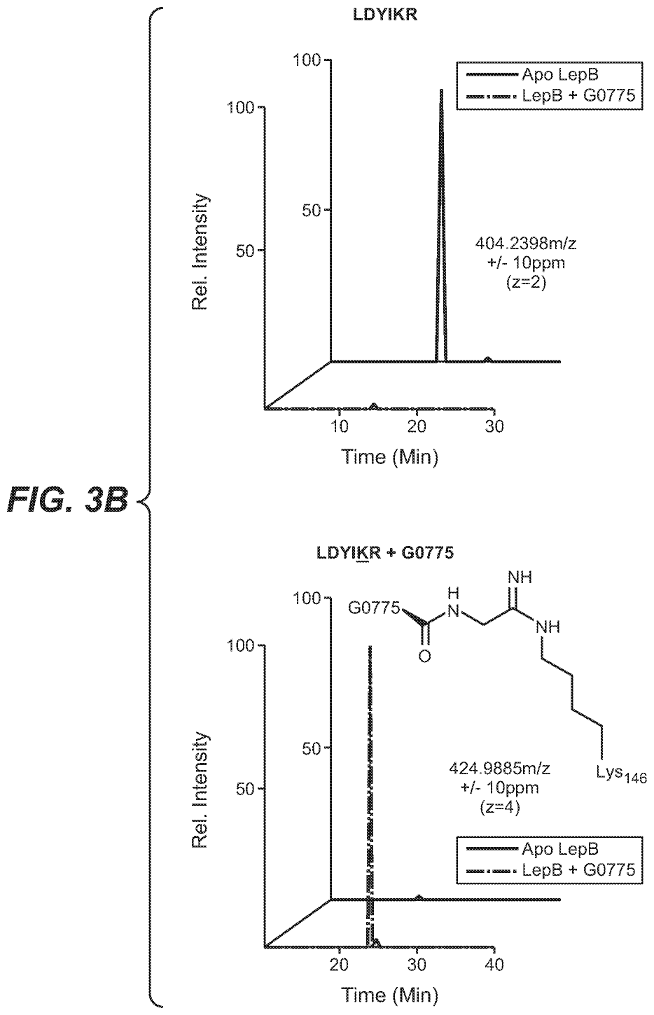

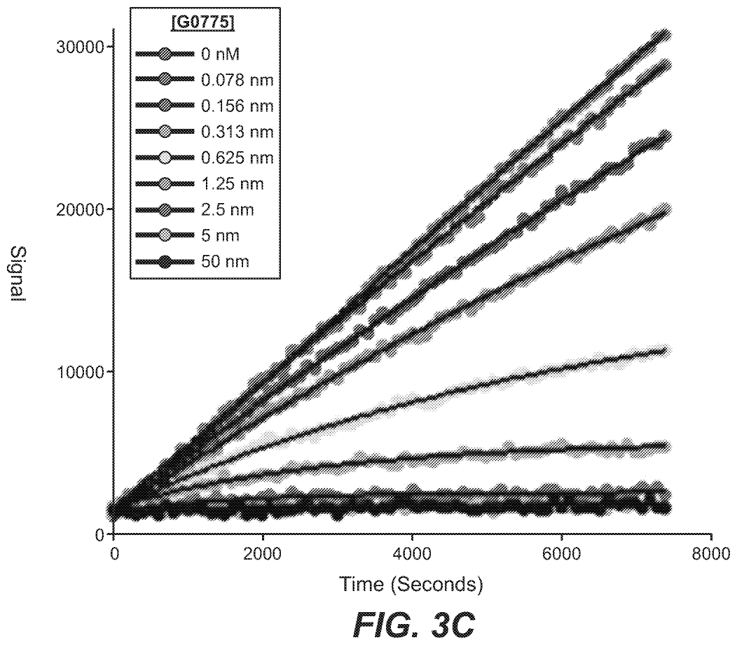

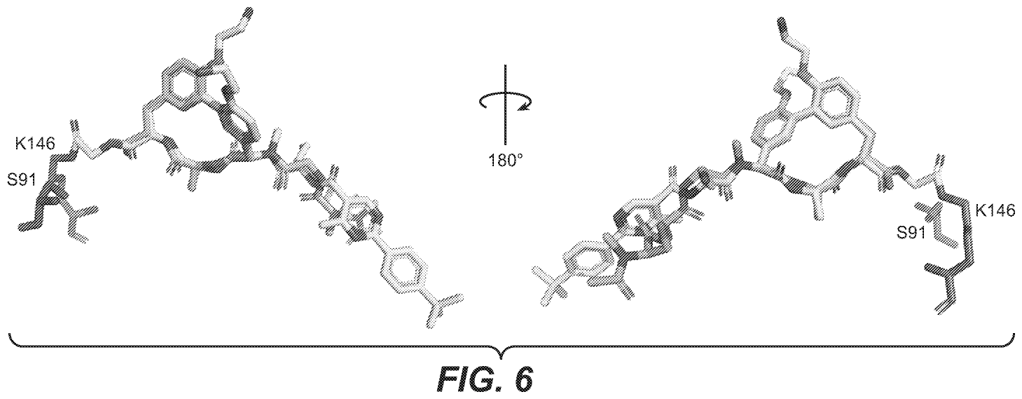

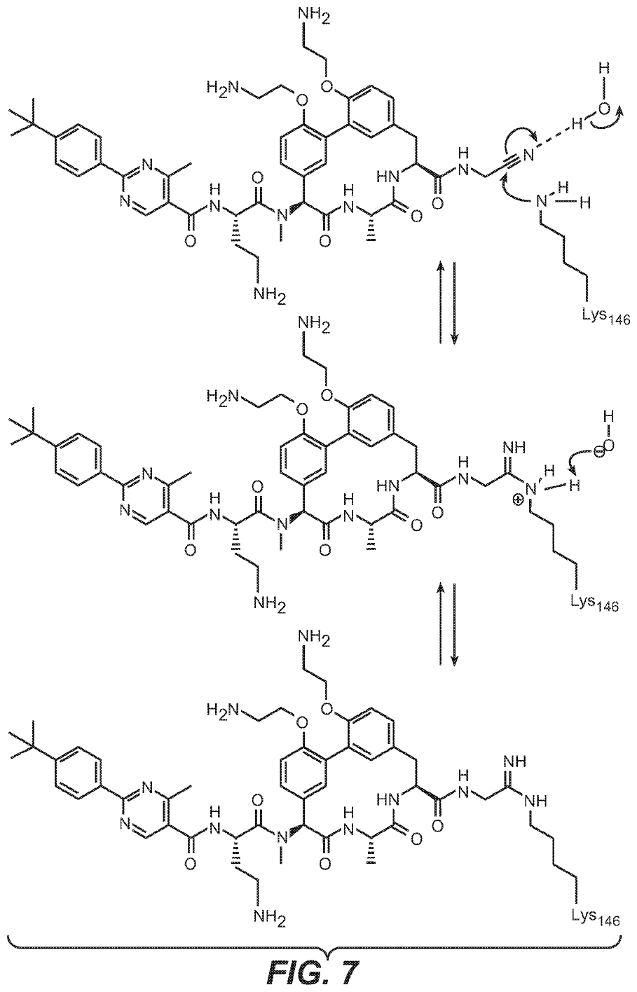

To test the hypothesis that resistance conferring LepB mutations alter residues that directly contact the inhibitor, the co-crystal structure of LepB in complex with G0775 was solved to 2.8 Å resolution. Mapping the evolved mutations onto the E. coli LepB crystal structure confirms that these mutations lie with within the substrate binding groove and closely overlay the G0775 binding site (FIG. 2B). Indeed, all of the resistance-conferring mutations lie within 4.5 Å of the bound inhibitor, and in total, mutations were identified in eight of the twenty contact residues. Overall the LepB-G0775 structure reveals that the core and backbone amide heteroatoms of the inhibitor bind nearly identically to previously determined structures of arylomycins bound to LepB¹³¹⁴ (FIG. 6). Surprisingly, the aminoacetonitrile warhead on G0775 does not engage the catalytic serine (S91), but instead, interacts with the nitrogen of the catalytic lysine (K146) (FIG. 3A). This is consistent with the formation of a covalent amidine via direct nucleophilic attack of the electrophilic nitrile by the catalytic lysine, an inversion of the typical roles of the active site dyad (FIG. 7), and to our knowledge, is the first report of amidine formation between an inhibitor and its target protein. To confirm the unique nature of this covalent interaction, LepB was incubated with an excess of G0775 overnight, digested with trypsin, and subjected the digest to LC-MS analysis (FIG. 3B). The resulting fragments confirm the formation of an adduct of the expected molecular weight between G0775 and a LDYIKR peptide fragment (SEQ ID NO: 5) that includes the catalytic lysine residue 146. This mechanism of covalent protease inhibition is unique and adds a new tool to the rapidly expanding field of covalent enzyme inhibitors. Detailed kinetic enzymology studies of full-length LepB embedded in a detergent micelle were performed to quantify the rate of covalent bond formation with the catalytic lysine. The data indicate a very tight prechemistry binding of the molecule into the substrate binding site (K_{I}=0.44 nM) followed by irreversible inactivation (k_{inact}=0.0007 l/s; k_{inact}/K_{I}=1,590,909 M⁻¹ s⁻¹) (FIG. 3C) confirming the affinity of G0775 for LepB.

### Example 5. G0775 Penetrates the Outer Membrane

The potent antibacterial activity G0775 suggests that it is able to penetrate the Gram-negative OM and thereby access the LepB active site. To better understand the efficiency with which G0775 enters the periplasmic space, the potency of G0775 against a hyper-permeable E. coli strain (imp4213) that harbors a defect in the OM biogenesis pathway was determined and a 30-fold increase in potency relative to wild type E. coli. EDTA mediated chelation of the divalent cations that are crucial for maintaining OM stability induces a similar MIC shift (Table 3) was observed. This degree of OM penetrance is in the range observed for Gram-negative active beta lactam antibiotics that cross the OM via aqueous channels called porins, raising the possibility that G0775 accesses the periplasm through a similar mechanism. To directly assess whether G0775 accesses the periplasmic space by permeating through porins, its activity against wild type E. coli K-12 MG1655 and its isogenic mutants lacking the classical porins OmpC, OmpF or both was examined. G0775 displays identical potency against all four strains (Table 3), consistent with its high molecule weight (890 g/mol), which is above the range typically considered compatible with porin mediated entry. The data suggests that G0775 permeation into the periplasmic space proceeds through alternative porins or alternatively through a distinct porin-independent mechanism.

### Example 6. G0775 is Efficacious In Vivo Against Clinically Relevant Gram-Negative Bacteria

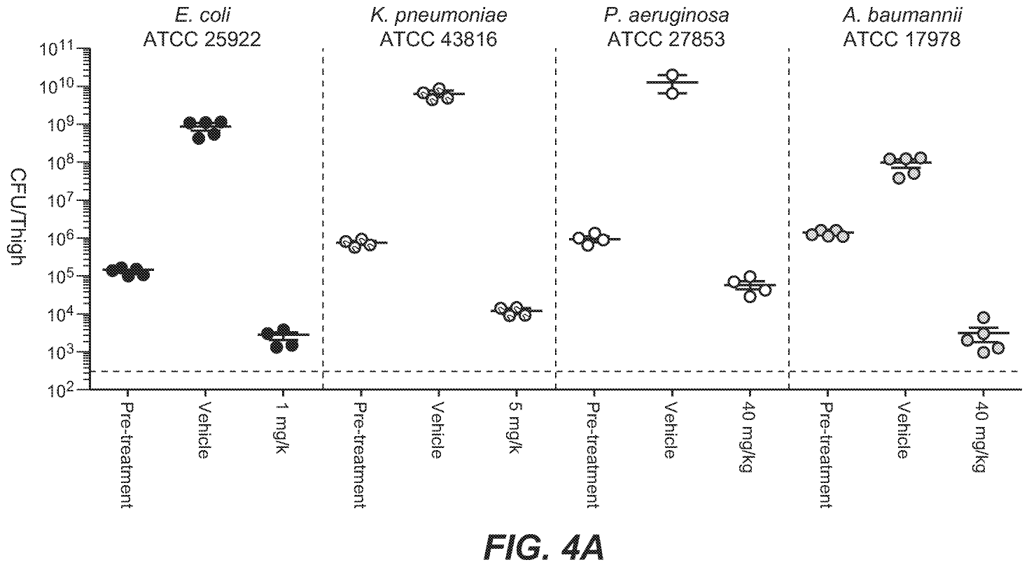

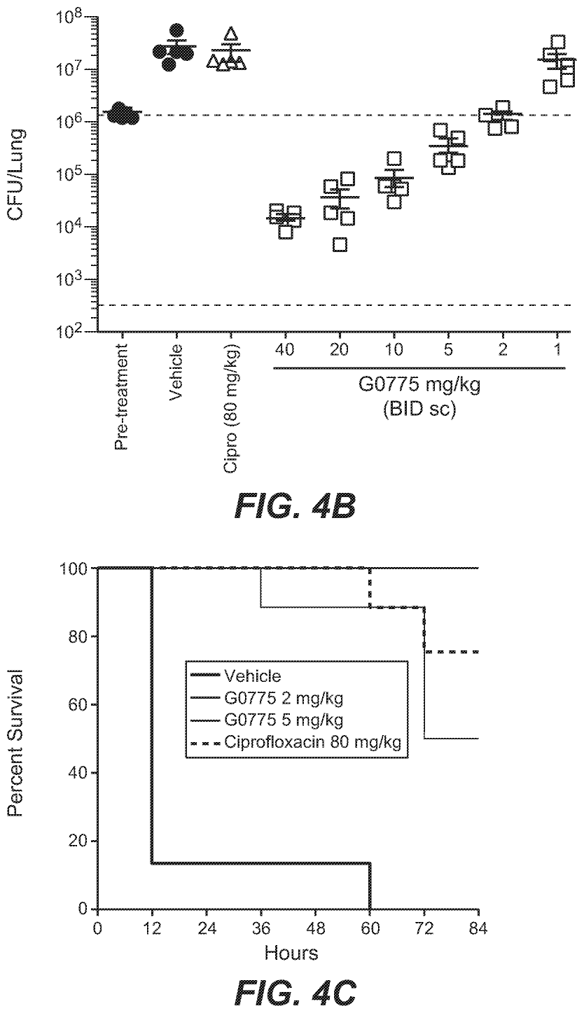

The potent in vitro activity of G0775 in the presence of 50% serum or 1% lung surfactant (Table 3), suggested that this antibiotic could be used to treat serious systemic and pulmonary infections, so we were motivated to determine its in vivo efficacy. Multiple pre-clinical infection models was tested with different pathogenic Gram-negative bacteria. The in vivo efficacy of G0775 in the murine neutropenic thigh infection model was first evaluated, which assesses intrinsic antibacterial activity in isolation from the innate immune system and has proven to translate well from preclinical species into humans. G0775 demonstrates potent bactericidal activity (>2-log decrease in CFU) against the E. coli strain ATCC 25922 and K. pneumoniae strain ATCC 43816 at 1 mg/kg and 5 mg/kg, respectively (FIG. 4A).

G0775 was next tested against P. aeruginosa 27853 and A. baumannii 17978 in the same neutropenic thigh model. Again, G0775 was efficacious but required a higher concentration of antibiotic, consistent with the higher MIC values for these pathogens (FIG. 4A). Using a lung infection model, it was possible able to assess the ability of G0775 to treat MDR bacterial infections in a pulmonary setting. In these experiments, the drug resistant CDC 106 (Table 2) was used, and bacteriostatic activity with G0775 at 2 mg/kg and bactericidal activity at 20 mg/kg was observed (FIG. 4B). These results confirm that this new class of antibiotic is able to overcome all of the resistance mechanisms inherent to CDC 0106 whether the bacteria are growing in vitro or in vivo. Finally, the ability of G0775 to protect mice from a lethal challenge of K. pneumoniae strain ATCC 43816 was examined in a mucin peritonitis model. G0775 effected a dose-dependent increase in survival with 5 mg/kg BID delivered on day 1 of the infection able to maintain full animal viability out to 84 hours (FIG. 4C). In total these data demonstrate the potential of G0775 to treat multiple types of infections caused by a variety of wild type and MDR Gram-negative bacteria.

### Example 7. Synthetic Methods for Compounds G3031 and G0775

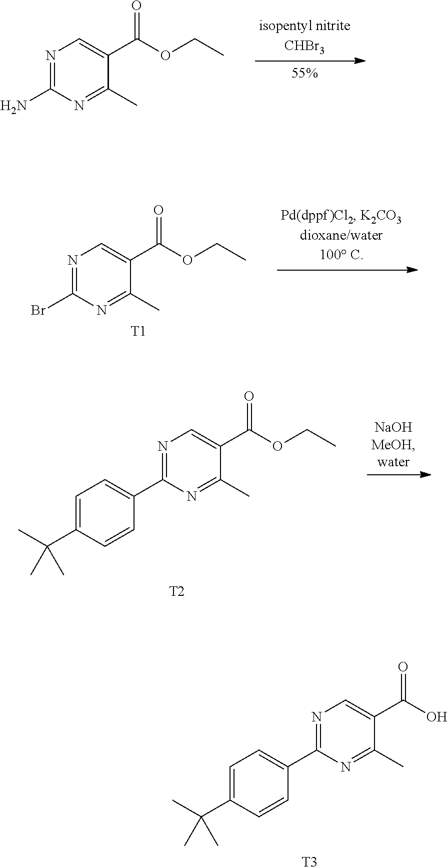

To a solution of ethyl 2-amino-4-methylpyrimidine-5-carboxylate (4.0 g, 22 mmol) in CHBr₃ (66 mL) was added isopentyl nitrite (44 mL) and the mixture was stirred at 85° C. for 4 h. The volatiles were removed and the residue was taken up by EtOAc (100 mL), which was washed by brine (100 mL×2). The organic layer was dried over Na₂SO₄, concentrated and the residue was purified by silica gel flash column to give ethyl 2-bromo-4-methylpyrimidine-5-carboxylate T1 (3.0 g, 55.5% yield) as a white solid. ¹H NMR (400 MHz, CDCl₃): δ 8.93 (s, 1H), 4.41 (q, J=7.2 Hz, 2H), 2.82 (s, 3H), 1.41 (t, J=7.0 Hz, 3H).

A solution of 4-t-butylbenzeneboronic acid (944 mg, 5.3 mmol), potassium carbonate (1.69 g, 12.3 mmol), 1,1′-bis(diphenylphosphino)ferrocene palladium dichloride (299 mg, 0.41 mmol) and T1 (1.0 g, 4.1 mmol) in toluene (10 mL) and water (2 mL) was stirred at 110° C. for 16 h under N₂. The residue was taken up in EtOAc (200 mL) and washed with water and brine (200 mL each). The organic layer was dried over MgSO₄ and concentrated. The residue was purified by flash column chromatography to afford methyl 4-(4-tert-butylphenyl)-2-methyl-benzoate T2 (920 mg, 76% yield) as a colorless oil. ¹H NMR (400 MHz, CDCl₃) δ 9.21 (s, 1H), 8.44 (d, J=8.4 Hz, 2H), 7.53 (d, J=8.4 Hz, 2H), 4.43 (q, J=7.2 Hz, 2H), 2.89 (s, 3H), 1.44 (t, J=7.2 Hz, 3H), 1.38 (s, 9H).

A solution of T2 (920 mg, 3.1 mmol) and sodium hydroxide (620 mg, 15.5 mmol) in methanol (15 mL) and water (15 mL) was stirred at 85° C. for 2 h. After that, the mixture was adjusted to pH=3-4 with 1N HCl, which was extracted with EtOAc (200 mL×2). The combined organic layers were washed with brine (200 mL×2), dried over Na₂SO₄, concentrated to give 2-(4-tert-butylphenyl)-4-methyl-pyrimidine-5-carboxylic acid T3 (800 mg, 96% yield) as a white solid. LCMS (5-95AB_1.5 min): t_{R}=0.806 min, [M+H]⁺=270.8

The synthesis of tyrosine boronic acid S6 has previously been and was carried out with the amine protected as a Boc carbamate rather than the Cbz. Spectral data for S6 was identical to that previously described. The arylomycin core S18 was then constructed using small variations to the chemistry previously described.

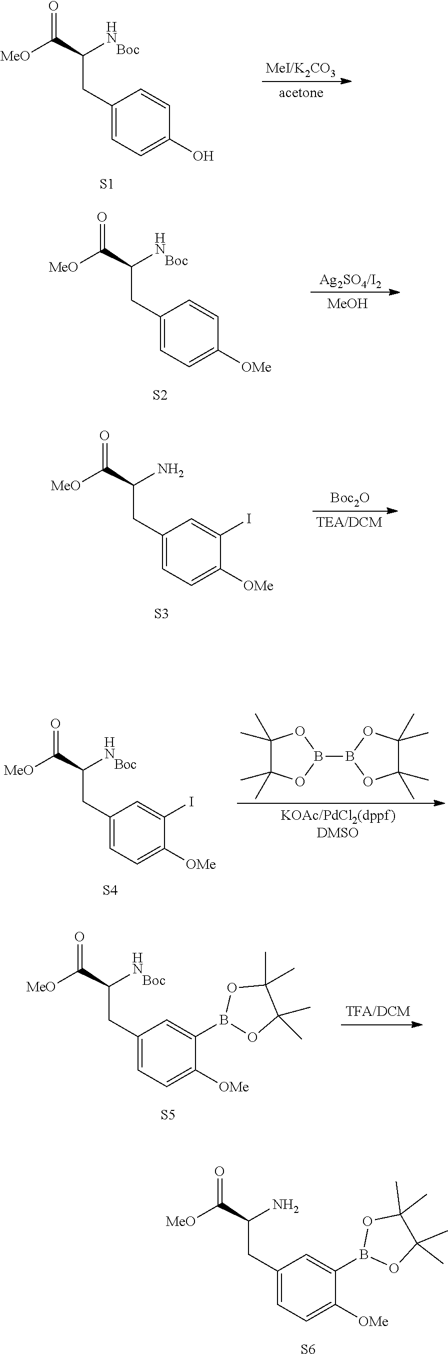

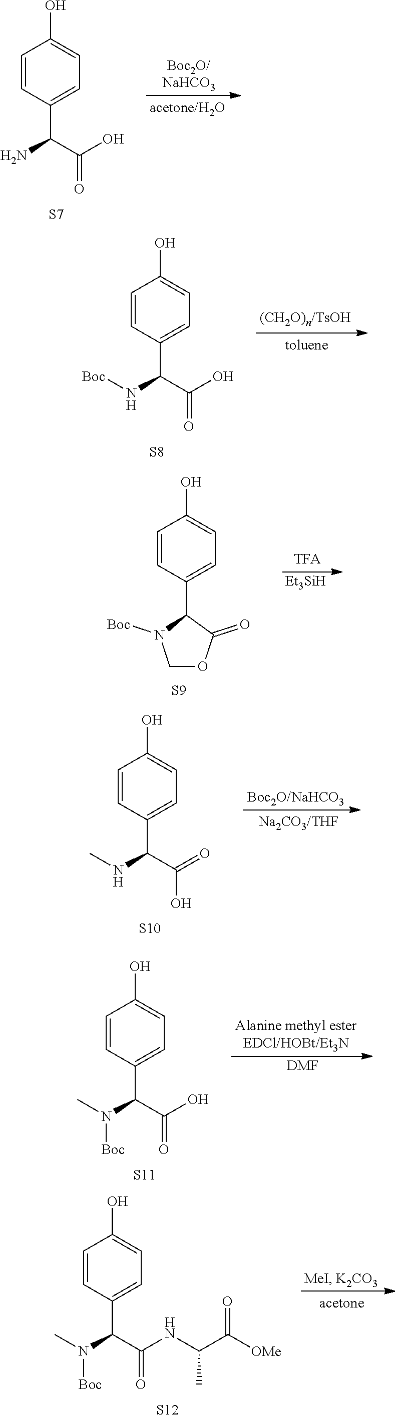

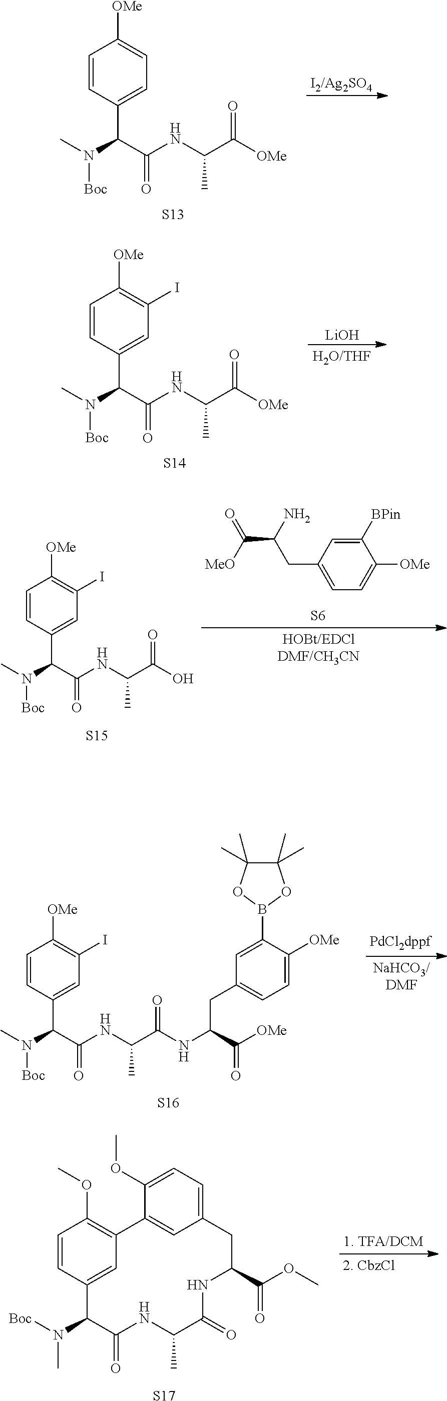

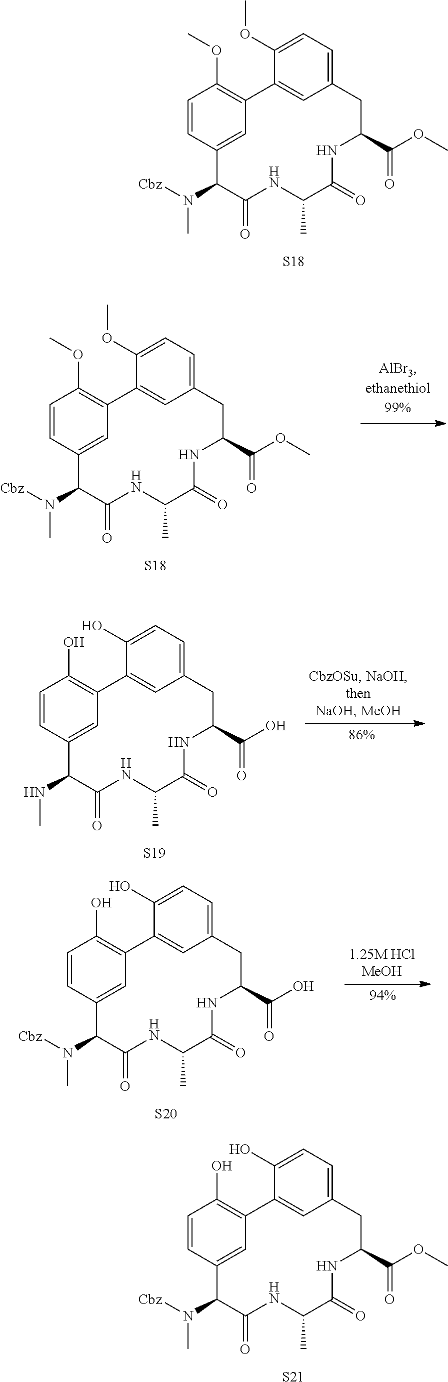

To a solution of Compound S18 (5.0 g, 11.0 mmol) in EtSH (116 mL), AlBr₃ in CH₂Br₂(1 mol/L, 165 mL, 165 mmol) was added slowly at 0° C. under an atmosphere N₂. The mixture was stirred for 18 h. The volatiles were removed under reduced pressure and the residue was quenched by addition of water (50 mL), which was further washed by DCM (20 mL×3). The aqueous layer was purified by prep-HPLC(acetonitrile 1-20%/0.1% TFA in water) to give the fully deprotected arylomycin core S19 (4.5 g, 99.2% yield) as a white solid.

To a solution of S19 (4.7 g, 8.9 mmol) in 1,4-dioxane/H₂O (9:1, 165 mL) was added 1 N NaOH dropwise until the pH was approximately 11. A solution of Cbz-OSu (6.66 g, 26.7 mmol) dissolved in 1,4-dioxane (50 mL) was then added. After stirring for 1 h, NaOH (1.07 g, 26.7 mmol) was then added to the reaction followed by MeOH (60 mL). This resulting mixture was allowed to stir for 20 mins. To the reaction was then added dilute citric acid (10% v/v, 50 mL), the aqueous layer was extracted with EtOAc (3×150 mL) and the combined organic layers were washed with brine (3×100 mL), dried over Na₂SO₄ and concentrated to give the crude product. The residue was diluted with DCM (50 mL) and the suspension was filtered to give desired material (3.2 g). The DCM phase was concentrated and the residue was purified by silica gel column (eluting 10˜20% methanol in EtOAc) to give a second crop of the desired material (1.0 g). The combined batches gave S20 (4.2 g, 86.1% yield) as a white solid.

To S20 (4.3 g, 7.85 mmol) was added a solution of 1.25M HCl in MeOH (128 mL) and the reaction was stirred at 0° C. The volatiles were removed and the residue was re-dissolved in EtOAc (200 mL), which was washed by saturated NaHCO₃ solution and brine (200 mL each). The organic layer was concentrated to afford S21 (4.15 g, 94.1% yield) as a white solid, which was used directly in the next step.

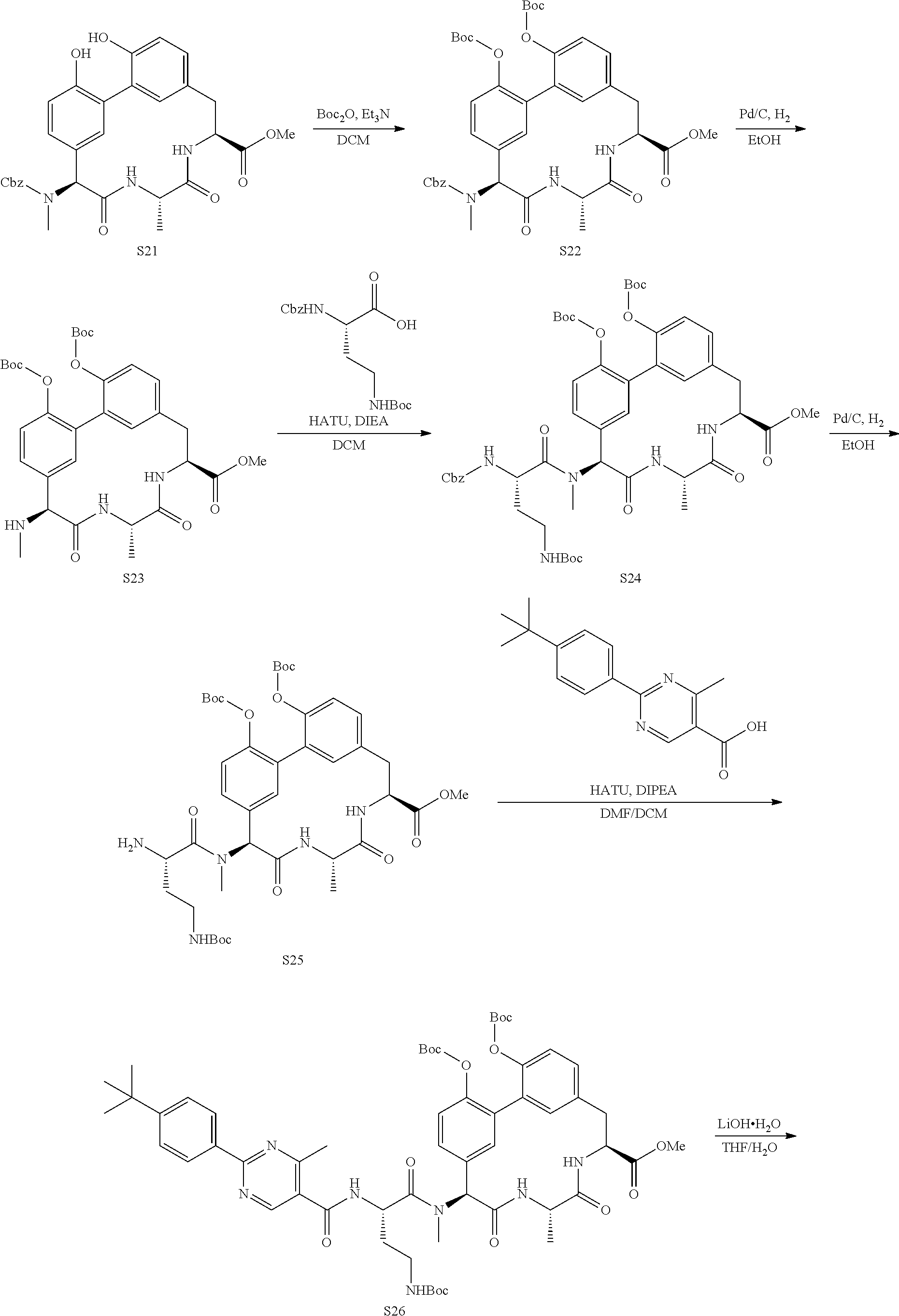

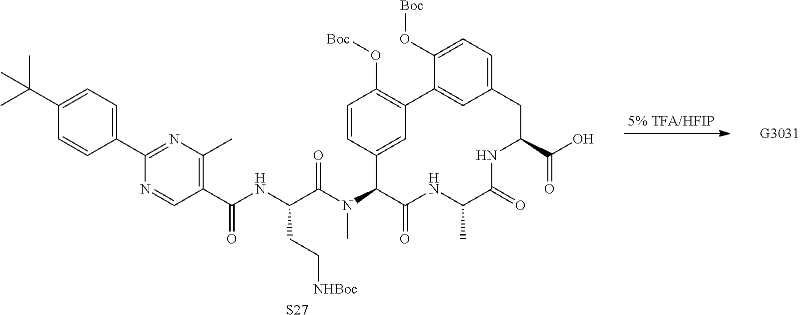 [SMILES: Cc1nc(-c2ccc(C(C)(C)C)cc2)ncc1C(=O)N[C@@H](CCNC(=O)OC(C)(C)C)C(=O)N(C)[C@@H]1C(=O)N[C@@H](C)C(=O)N[C@H](C(=O)O)Cc2ccc(OC(=O)OC(C)(C)C)c(c2)-c2cc1ccc2OC(=O)OC(C)(C)C ; "5% TFA/HFIP" ; "S27"]

To a solution of S21 (150 mg, 0.27 mmol), Et₃N (324 mg, 3.2 mmol) in DCM (5 mL) was added Boc₂O (583 mg, 2.7 mmol). The mixture was stirred at 30° C. for 3 h. The volatiles were concentrated and the residue was added to a mixture of water (50 mL) and EtOAc (50 mL). The organic phase was washed with brine (2×50 mL), dried over Na₂SO₄ and concentrated. The residue was purified on silica gel, eluting with EtOAc:EtOH:petroleum ether=3:1:8, to afford S22 (200 mg, 98.3% yield) as a white solid.

LCMS (5-95AB_1.5 min): t_{R}=0.893 min, [M+Na]⁺=784.2

To a solution of S22 (200 mg, 0.25 mmol) in EtOH (12 mL), 10% Pd/C (56 mg, 0.05 mmol) was added. The mixture was stirred for 2 h at 25° C. under hydrogen (15 psi). After filtration and the filtrate was concentrated in vacuo to give the product of S23 (157 mg, 99% yield) as a white solid.

A solution of S23 (165 mg, 0.26 mmol), DIEA (102 mg, 0.79 mmol) and (S)-2-(((benzyloxy) carbonyl)amino)-4-((tert-butoxycarbonyl)amino)butanoic acid (111 mg, 0.32 mmol) in DCM (5 mL) was stirred for 5 min at 0° C., followed by the slow addition of HATU (110 mg, 0.29 mmol) at 0° C. The resulting mixture was stirred for 10 min at 0° C., gradually warmed to 25° C. and stirred for 1 h at 25° C.

The reaction mixture was concentrated and the residue was added to a mixture of water (40 mL) and EtOAc (40 mL); the organic phase was washed with (3×40 mL) saturated brine solution, dried over Na₂SO₄ and concentrated. The residue was purified by prep-TLC (5% MeOH in DCM) to afford S24 (165 mg, 65% yield) as a white solid. LCMS (5-95AB_1.5 min): t_{R}=0.930 min, [M+Na]+=984.5

To a solution of S24 (165 mg, 0.17 mmol) in EtOH (12 mL), 10% Pd/C (36.5 mg, 0.03 mmol) was added. The mixture was stirred for 2 hr at 25° C. under hydrogen (15 psi). After filtration, the filtrate was concentrated in vacuo to give S25 (140 mg, 99% yield) as a white solid.

A solution of 2-(4-(tert-butyl)phenyl)-4-methylpyrimidine-5-carboxylic acid (27 mg, 0.10 mmol), HATU (37 mg, 0.10 mmol) and DIEA (27 mg, 0.21 mmol) in DMF (0.50 mL) was stirred for 5 min at 0° C., followed by the slow addition of a solution of S25 (77 mg, 0.09 mmol) in DCM (5 mL) at 0° C. The resulting mixture was stirred for 10 min at 0° C., gradually warmed to 25° C. and stirred for 1 h at 25° C. The reaction mixture was concentrated and the residue was added to a mixture of water (40 mL) and EtOAc (40 mL); the organic phase was washed with saturated brine solution (3×30 mL), dried over Na₂SO₄ and concentrated. The residue was purified by prep-TLC (5% MeOH in DCM) to afford S26 (96 mg, 98% yield) as a white solid. LCMS (5-95AB_1.5 min): t_{R}=1.177 min, [M+H]=1080.8

To a solution of S26 (96 mg, 0.09 mmol) in THF (5 mL) was added LiOHH₂O (7.5 mg, 0.18 mmol) in H₂O (1 mL). The mixture was stirred at 25° C. for 1 h. The mixture was added with H₂O (30 mL) and adjusted to pH=3 with 1M HCl, which was extracted with EtOAc (3×30 mL). The combined organic layers were washed with saturated brine solution (70 mL), dried over Na₂SO₄ and concentrated to give S27 (90 mg, 95% yield) as a white solid. LCMS (5-95AB_1.5 min): t_{R}=1.077 min, [M+H]⁺=1066.1

S27 (90 mg, 0.08 mmol) was added to 5% TFA in HFIP (5 mL) and the mixture was stirred for 2 h at 25° C. The volatiles were removed and the residue was purified by prep-HPLC (acetonitrile: 18-28%/0.225% FA in water) to give G3031 (20.3 mg, 30% yield) as a white solid. LCMS (5-95AB_1.5 min): t_{R}=0.68 min, [M+H]⁺=766.1. ¹H NMR (400 MHz, CD₃OD): 8.78 (s, 1H), 8.38 (brs, 2H), 7.52 (brs, 2H), 7.17-6.73 (m, 5H), 6.60-6.54 (m, 1H), 5.18 (brs, 1H), 4.65-4.58 (m, 2H), 3.34-3.32 (m, 2H), 3.21-3.10 (m, 2H), 2.98 (s, 3H), 2.33 (s, 3H), 2.39-2.27 (m, 1H), 2.25-2.14 (m, 1H), 1.39 (s, 9H), 1.38 (d, J=7.2 Hz, 3H).

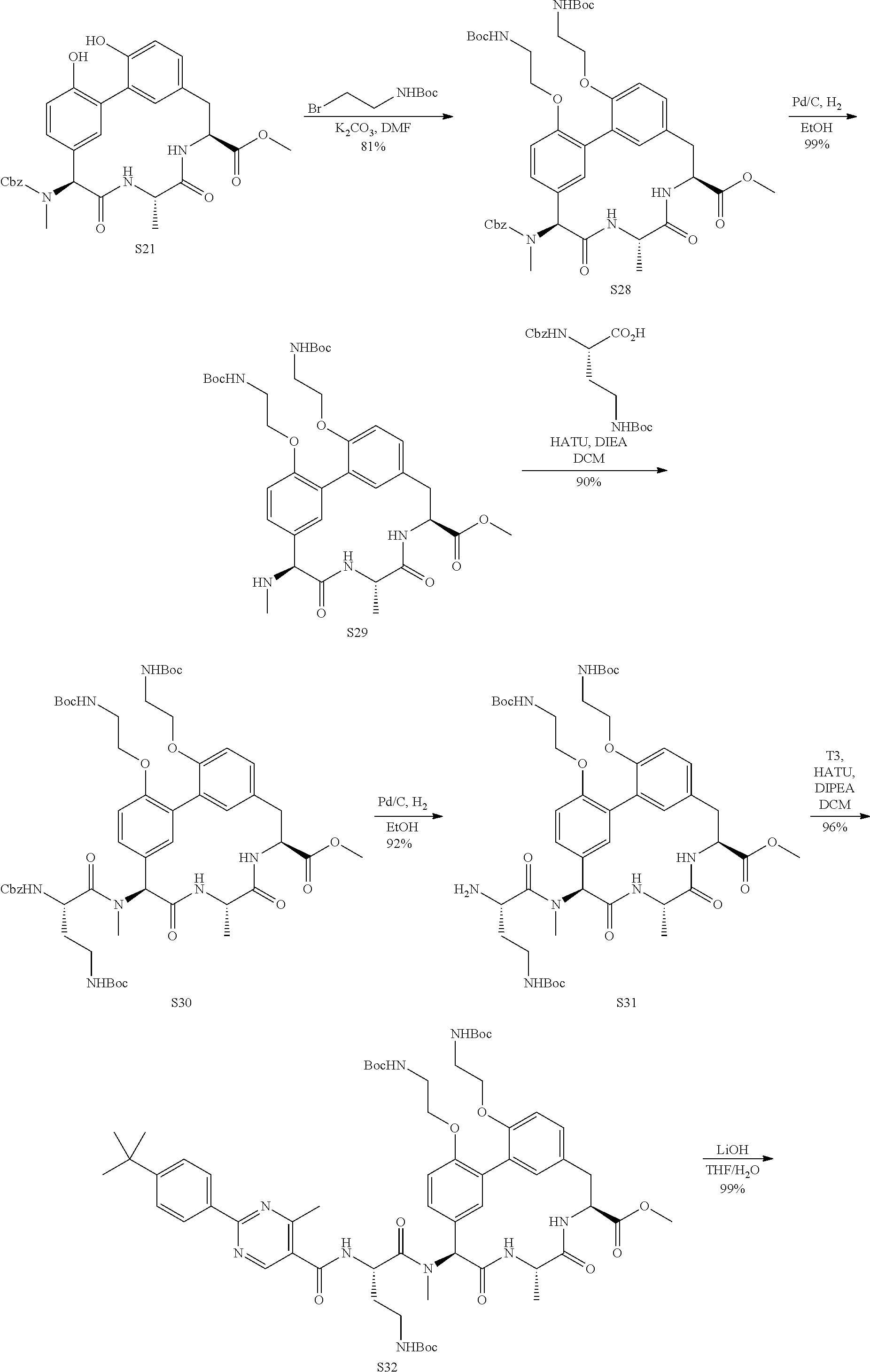

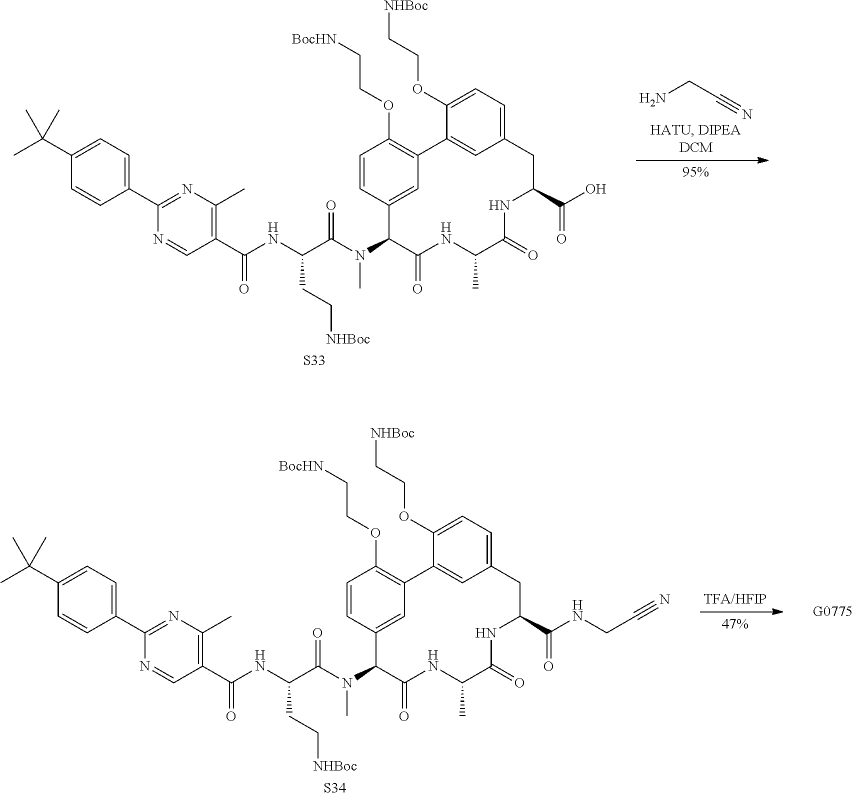

To a solution of S21 (3.9 g, 6.94 mmol) and K₂CO₃ (14.4 g, 104 mmol) in DMF (50 mL) was added tert-butyl 2-bromoethylcarbamate (15.6 g, 69.5 mmol) at 0° C. The mixture was stirred at room temperature for 48 h. The mixture was filtered and the filtrate was diluted with EtOAc (500 mL). The EtOAc layer was washed with brine (2×400 mL), dried over Na₂SO₄, concentrated and purified by chromatography on silica (solvent gradient: 0-60% EtOAc in petroleum ether) to afford S28 (4.8 g, 81.5% yield) as a white solid.

To a solution of S28 (4.8 g, 5.7 mmol) in EtOH (100 mL), 10% Pd/C (1.26 g, 1.18 mmol) and a drop of 30% NH₄OH solution were added at room temperature. The reaction mixture was stirred for 1 h at the same temperature under hydrogen atmosphere (15 psi). The filtrate was then concentrated to afford S29 (4.0 g, 99% yield) as a white solid.

A mixture of S29 (2.2 g, 3.1 mmol), (2S)-2-(benzyloxycarbonylamino)-4-(tert-butoxycarbonylamino)butanoic acid (1.4 g, 4.0 mmol) and DIEA (1.6 mL, 9.3 mmol) in DCM (25 mL) was stirred at 0° C. for 10 min, followed by the addition of HATU (2.3 g, 6.2 mmol). The reaction mixture was stirred at the same temperature for 1 h. The reaction was concentrated to dryness and the residue was partitioned between EtOAc (150 mL) and H₂O (150 mL). The organic layer was washed with 10% Na₂CO₃ solution and brine (150 mL each), dried over Na₂SO₄ and concentrated. The residue was purified on silica gel column to give S30 (2.9 g, 90% yield) as a white solid. LCMS (5-95AB_1.5 min): t_{R}=0.969 min, [M+H]⁺=1048.5

To a solution of S30 (2.0 g, 1.9 mmol) in EtOH (100 mL), 10% Pd/C (600 mg, 0.57 mmol) and a drop of 30% NH₄OH was added at room temperature. The reaction mixture was stirred for 1 h at the same temperature under hydrogen atmosphere (15 psi). The filtrate was then concentrated to afford S31 (1.6 g, 92% yield) as a white solid.

A solution of T3 (399 mg, 1.48 mmol), HATU (749 mg, 1.97 mmol) and DIEA (516 μL, 2.95 mmol) in DMF (2 mL) was stirred at 0° C. for 15 min, followed by the addition of a solution of S31 (900 mg, 0.98 mmol) in DCM (10 mL). The mixture was stirred and maintained at 0° C. for 1 h. The reaction mixture was then concentrated to dryness and the residue was partitioned between EtOAc (100 mL) and water (100 mL). The organic layer was washed with 10% Na₂CO₃ solution and brine (100 mL each), dried over Na₂SO₄ and concentrated. The residue was purified by silica gel column to give S32 (1.1 g, 96% yield). LCMS (5-95AB_1.5 min): t_{R}=0.947 min, [M+H]=1166.7.

To a solution of S32 (1.1 g, 0.94 mmol) in THF (10 mL) was added lithium hydroxide monohydrate (90 mg, 3.8 mmol) in water (10 mL) at 0° C. and the mixture was gradually warmed up to room temperature while stirring and stirred at the same temperature for 1 h. The mixture was added with 1M KHSO₄ solution until pH to 5, which was extracted by EtOAc (40 mL×3). The combined organic layers were washed with brine (100 mL×2), dried over Na₂SO₄ and concentrated to give S33 (1.07 g, 99% yield) as a white solid. LCMS (5-95AB_1.5 min): t_{R}=1.054 min, [M+H]=1153.1.

To a solution of S33 (970 mg, 0.84 mmol), amino acetonitrile hydrochloride (156 mg, 1.68 mmol) and DIEA (441 μL, 2.5 mmol) in DCM (12 mL) was added a solution of HATU (640 mg, 1.68 mmol) in DMF (2 mL) at 0° C. The reaction was stirred at the same temperature for 1 h. The reaction mixture was concentrated to dryness and the residue was partitioned between EtOAc (100 mL) and water (100 mL). The organic phase was washed with 10% Na₂CO₃ solution and brine (100 mL each), dried over Na₂SO₄ and concentrated. The residue was purified by silica gel column to give S34 (950 mg, 95% yield) as a white solid. LCMS (5-95AB_1.5 min): t_{R}=1.057 min, [M+H]⁺=1190.9.

S34 (950 mg, 0.80 mmol) was added to 5% TFA in hexafluoroisopropanol (v/v, 20 mL) and the mixture was stirred at room temperature for 1 h. The volatiles were removed under reduced pressure and the resulting crude solid was re-dissolved in acetonitrile (5 mL), and then neutralized with solid NaHCO₃. The filtrate was then purified by HPLC to afford G0775 (331 mg, 47% yield) as a white solid. LCMS (Method 5-95 AB, ESI): t_{R}=0.724 min, [M+H]⁺=890.5. H NMR (400 MHz, CD₃OD): δ 8.78 (s, 1H), 8.36 (d, J=8.4 Hz, 2H), 7.55 (d, J=8.4 Hz, 2H), 7.32 (d, J=8.8 Hz, 1H), 7.25-7.15 (m, 2H), 7.08 (d, J=8.8 Hz, 1H), 6.90 (s, 1H), 6.79 (s, 1H), 6.39 (s, 1H), 5.19-5.16 (m, 1H), 4.80-4.70 (m, 2H), 4.28-4.15 (m, 4H), 4.11 (s, 2H), 3.20-3.10 (m, 8H), 2.95 (s, 3H), 2.65 (s, 3H), 2.35-2.05 (m, 2H), 1.39 (s, 9H), 1.36 (d, J=6.4 Hz, 3H).

### Example 8. LepB Protease Domain Forms an Irreversible Covalent Bond with G0775

G0775 binds LepB protease domain to form an irreversible covalent bond with catalytic lysine 146 (see FIGS. 3A-3C). LCMS detection of LDYIKR LepB peptide fragment (SEQ ID NO: 5) after tryptic digest following incubation of LepB with G0775 shows the unmodified peptide is only detected in the absence of G0775 incubation while the LDYIKR-G0775 peptide (SEQ ID NO: 5) adduct is only detected subsequent to LepB incubation with G0775 (FIG. 3B). The nonlinear inactivation curves in LepB kinetic enzyme assays in the presence of the indicated concentration of G0775 indicate time-dependent inhibition consistent with covalent bond formation. K_{inact} (0.0007±0.0002 s⁻¹) and K_{I} (0.44±0.15 M⁻¹ s⁻¹) were measured from three independent experiments and the data points shown are averages of four replicates from a single experiment.

### Example 9. Overlay of LepB-G0775 and LepB-Arylomycin from PDB 1T7D. Comparison of G0775 and Arylomycin

As depicted in FIG. 6, the catalytic lysine 146 is covalently bound to the nitrile warhead while the serine 91 nucleophile remains unbound. For simplicity, the LepB protein has been removed from each co-structure. The comparison indicates that the macrocyclic core of G0775 maintains high conservation to the parent arylomycin macrocycle and makes very similar interactions with the protein.

### Example 10. Proposed Mechanism of Covalent Amidine Bond Formation Between G0775 Nitrile and Lysine 146

As shown in FIG. 7, the general base (lysine 146) involved in substrate proteolysis functions instead as a nucleophile to attack the nitrile warhead.

### Example 11. Electron Density and Space Groups of the G0775-LepB Co-Structure

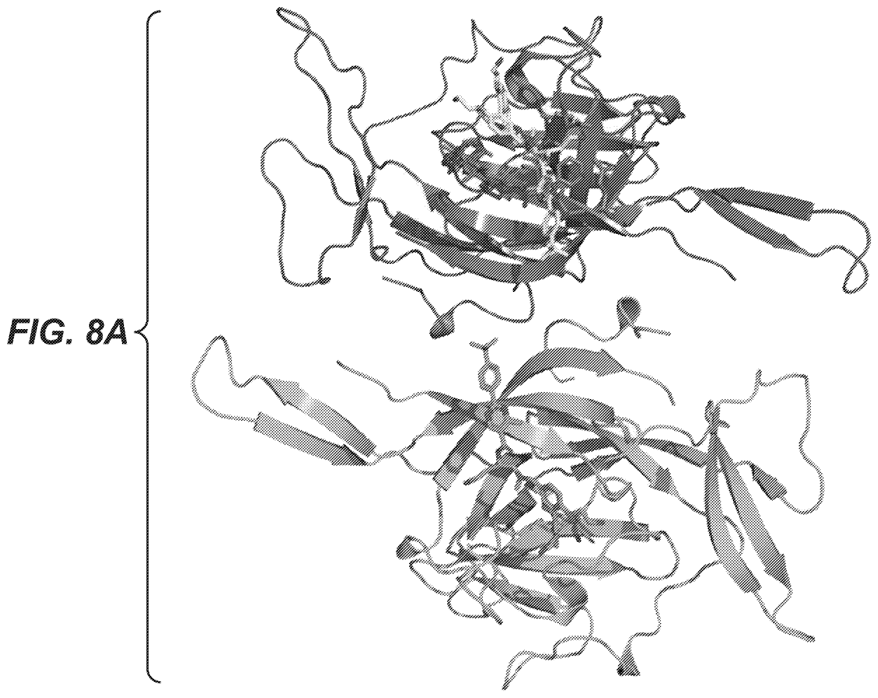

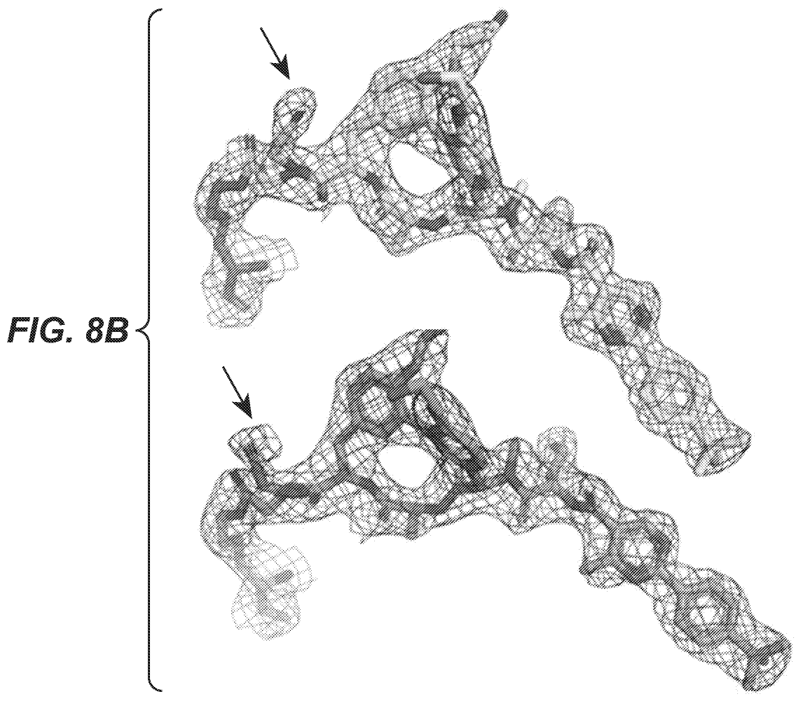

As shown in FIG. 8, The electron density indicates partial occupancy for an unreacted, non-covalently bound form of G0775 in each active site in the asymmetric unit. The appearance of this non-covalent form of the inhibitor is thought to be an artifact that is the result of radiation damage during X-ray data collection since MS analysis indicated that reaction between LepB and G0775 goes to completion.

### Example 12. Optimized Arylomycins are a New Class of Gram-Negative Antibiotics

Multidrug resistant bacteria are spreading at alarming rates, and despite extensive efforts, no new antibiotic class with activity against Gram-negative bacteria has been approved in over fifty years. The most successful Gram-negative antibiotics are derivatives of natural products. Chemical optimization of the arylomycin class of natural products with weak activity and limited spectrum into G0775, a molecule with potent, broad-spectrum Gram-negative activity is described herein. This modified natural product functions by inhibiting the essential bacterial type I signal peptidase, but does so using a novel mechanism. It is bactericidal against contemporary multidrug resistant Gram-negative clinical isolates in vitro and in multiple in vivo infection models. These findings demonstrate that optimized arylomycin analogues like G0775 are poised to translate into novel therapies to address the growing threat of multidrug resistant Gram-negative infections.

### Introduction

The discovery and development of multiple classes of safe and efficacious antibiotics has dramatically reduced mortality from bacterial infections, but the overuse and misuse of these same antibiotics—both in medicine and in agriculture—has driven the rapid evolution and dissemination of antibiotic resistance. The most acute threat of pan drug resistant untreatable infections is from the ‘ESKAPE’ pathogens, particularly the Gram-negative members of this group (Escherichia coli, Klebsiella pneumoniae, Pseudomonas aeruginosa, and Acinetobacter baumannii), which have a dual membrane envelope that prevents many antibiotics from accessing their targets. Despite significant effort, no novel class of antibiotic has been approved for this class of pathogens in over fifty years.

The arylomycins, a well-described class of bridged lipopeptides, inhibit the bacterial Type I signal peptidase (SPase), an essential membrane bound protease that employs an atypical serine/lysine dyad to cleave signal sequences from pre-proteins following their translocation across the cytoplasmic membrane. SPase has been pursued as an antibiotic target for nearly twenty years with the focus on developing agents active against Gram-positive bacteria, where the enzyme active site is exposed on the surface of the cell. In Gram-negative bacteria the SPase active site is located in the periplasmic space between the cytoplasmic and outer membranes, and was thought inaccessible to the arylomycins because their high molecular weight and lipophilicity would preclude penetration of the outer membrane (OM). Contrary to these expectations, modest activity of arylomycin A-C₁₆ has been reported against several Gram-negative species, but to date, no arylomycins with activity against the Gram-negative ESKAPE pathogens have been reported. The discovery of G0775, a synthetic arylomycin derivative with potent in vitro antibacterial activity against the Gram-negative ESKAPE pathogens is described herein. Interestingly, the physicochemical properties of G0775 remain outside the range currently considered desirable for potent Gram-negative activity, suggesting that it employs an atypical mechanism of OM penetrance. Crucially, highly multidrug resistant (MDR) pathogens that are resistant to nearly all available antibiotic therapies remain susceptible to G0775, and de novo resistance to G0775 occurs at a very low frequency. The potent in vitro activity of G0775 translates into robust in vivo efficacy in multiple infection models demonstrating the potential of these optimized natural products to address the growing clinical threat of antibiotic resistance.

### Discussion

The growth in MDR bacterial infections is a serious risk to global health and most antibacterial discovery efforts focus on reinvigorating existing classes of clinically approved antibiotics via chemical modifications. While this approach is critical and effective, the resulting molecules are eventually subject to cross resistance, which has evolved and disseminated in response to previously used members of their parent class. In contrast, an entirely new class of antibiotic would not be susceptible to existing resistance mechanisms. Due to its novel mechanism of action of inhibiting essential type I signal peptidase, G0775 bypasses existing resistance mechanisms and is fully active in vitro and in vivo against MDR Gram-negative strains, including hyper-resistant strains like the clinical isolate CDC 0106.

Potent whole cell activity of G0775 results from a combination of its relatively efficient penetration of the OM, its avoidance of efflux and its target affinity. It is of interest to decipher the role that each of the described chemical medications inherent to G0775 impacts each of these distinct activity barriers. While the unique covalent bond formed by G0775 directly influences target engagement, it is possible that the irreversible covalent bond also enhances the molecule's longevity in the perisplasmic space by disrupting its ability to be effluxed. Likewise, binding to the cytoplasmic membrane via the optimized N-terminal lipid anchor may further perturb this equilibrium while simultaneously functioning to optimally position the macrocyclic core and warhead within the SPase binding site. Finally, the appended ethylamines appear to have no direct interaction with LepB suggesting that these positive charges may increase the local concentration of the inhibitor in the periplasm, consistent with a recent report indicating that incorporation of basic amines can facilitate bacterial accumulation. Ultimately, the ability of G0775 to access SPase remains surprising given that its lipophilicity, high topological polar surface area and molecular weight fall outside of the range typically associated with Gram-negative OM penetrance.

The current understanding of the properties required for OM penetrance is primarily based on a relatively small number of antibiotic scaffolds, most of which utilize porin protein channels to cross the OM. Data provided herein suggest that other mechanisms may exist that allow G0775 to penetrate with sufficient efficiency to achieve potent whole cell activity. This OM penetrance pathway could contribute to the activity of other uncharacteristically large and polar inhibitors of periplasmic targets that exhibit Gram-negative antibacterial activity, including L27-11, globomycin, and Vancomycin aglycone analogues. While the details of this penetrance mechanism remain unclear, recent studies of OM biogenesis highlight the role of local membrane destabilization during the insertion of new beta-barrel proteins²⁸ or LPS monomers, and it is intriguing to speculate that this destabilization could potentially yield access points for molecules such as G0775. If so, it seems likely that a distinct set of rules may apply to large polar compounds that utilize a non-porin OM penetrance pathway, and ongoing studies of G0775 and related molecules may help elucidate these properties.

The broad spectrum and potent activity of G0775 combined with low vulnerability to spontaneous resistance and excellent preclinical efficacy suggest that optimized arylomycin analogues may represent a much needed novel class of Gram-negative antibiotic. Such a new mechanistic class of antibiotic has potential to reset the clock in the ongoing arms race with pathogenic bacteria to help postpone the prospect of a return to the pre-antibiotic era.

Data deposition: The sequence reported in this paper has been deposited in the GenBank database (world wide web at ncbi.nlm.nih.gov/genbank) (accession numbers CP022611-CP022613).

### Methods Summary

Minimum Inhibitory Concentration (MIC). MICs were determined by performing two-fold serial dilutions of compound in Mueller Hinton II cation-adjusted broth to a final volume of 0.1 m1 in round-bottom 96-well assay plates (Corning Life Sciences No 3788). Each well was inoculated with 5×10⁵ CFU/ml and incubated at 37° C. without agitation for 18 hours. Plates were scored by eye, and the lowest compound concentration preventing visible growth was determined to be the MIC.

Frequency of Resistance (FOR).

20 independent colonies from each bacterial species were scraped and used to establish an overnight culture. Bacteria were concentrated by centrifugation, and approximately 3×10⁹ CFUs were spread on cation adjusted Mueller Hinton agarose (MHA) plates containing G0775 at 2- to 8-times the MIC. The MHA plates were prepared from Mueller Hinton II cation adjusted broth (BBL™ 212322) and SeaKem LE Agarose (17 g/L; Lonza) as per manufactures instructions. Resistance frequency for each strain was calculated by dividing the number of colonies formed after a 48 hour incubation at 37° C. by the initial viable cell count.

Protein Fragmentation Mass Spectrometry.

Protein sequence was verified by enzymatic tryptic digestion (0.1 ug trypsin for 5 ug of LepB) followed by LC-MS/MS analysis on a UPLC (Waters) coupled to an Orbitrap Elite mass spectrometer (ThermoFisher). Samples were reduced with dithiothreitol and alkylated with iodoacetamide prior to digestion. Peptides were separated at 1 μl/min on a 45° C. heated Acquity M-Class BEH C18 column (Waters) over a 45 minute gradient. Mass spectrometry data was acquired with a top 15 data dependent method. Spectra were searched using the Mascot algorithm (Matrix Sciences) against a putative sequences database. Fixed modification, carbamidomethylation (57.02 Da), and variable modifications, oxidation (15.99 Da) and G0775 compound (899.46 Da), were included in the database search with a precursor mass tolerance of 20 ppm and a fragment ion tolerance of 0.8 Da. Acquired mass spectrometric data was analyzed using Xcalibur Qual Browser (ThermoFisher).

Protein Expression, Purification and G0775 Complex Formation.

BL21 (DE3) gold competent cells (Agilent) were transformed with a plasmid encoding the protease domain of E. coli LepB (Val77-His324),³⁰1. Cells were grown using autoinduction media (Terrific Broth, 72 hours at 16° C.), the cells were pelleted by centrifugation and resuspended into Buffer A (PBS, benzonase, cOmplete™ EDTA-free Protease Inhibitor Cocktail). The suspension was homogenized, passed through a microfluidizer and centrifuged at 13000 rpm for 40 minutes. The inclusion bodies were isolated and resuspended into Buffer B (0.5% Triton X-100, 10 mM EDTA, 20 mM Tris-HCl pH 7.4, 5 mM TCEP) and subjected to centrifugation again at 13000 rpm for 30 minutes. The pelleted inclusion bodies were washed 4 times with Buffer C (0.5% Triton X-100, 10 mM EDTA, 20 mM Tris-HCl pH 7.4) before being solubilized in Buffer D (6 M guanidine-HCl, 100 mM Tris-HCl pH 8 and cOmplete™ EDTA-free Protease Inhibitor Cocktail) at 4° C. overnight. The solution was centrifuged at 40000 rpm for 30 min and the supernatant was diluted to 0.1 OD_{280 nm} with Buffer E (4 M guanidine-HCl, 100 mM Tris-HCl pH 8). The solution was dialyzed overnight against 0.1M Tris pH 8.0, 0.4 M L-Arginine, 5 mM reduced glutathione and 0.5 mM oxidized glutathione. The following day, protein was further dialyzed against 25 mM Tris pH 8, 5 0 mM NaCl and 5% glycerol and the dialysis buffer was replaced every 12 h over the next 3 days.

The refolded material was loaded onto a MonoQ column 10/300GL (GE) equilibrated with Buffer E (25 mM Tris-HCl pH 8, 50 mM NaCl). The bound sample was washed with 10 CV of Buffer E and eluted using a gradient of Buffer F (25 mM Tris-HCl pH 8, 750 mM NaCl). Fractions containing protein were pooled and buffer exchanged into 25 mM Tris-HCl pH 8 and 150 mM NaCl. The purified protein was concentrated down to 1 mg/mL (35 μM) and 10×molar excess of G0775 (100 mM stock solution in DMSO) was added to the solution. The reaction was incubated overnight at 4° C., complex formation was monitored by LC-MS, and then concentrated to 20 mg/ml.

Crystallographic Methods.

Diffraction quality crystals of LepB in complex with G0775 were grown at 19° C. using hanging drop vapor diffusion method by mixing 1 μl protein at 20 mg/ml and 1 μl crystallization solution containing 30% PEG 300 and 0.1 M Sodium Acetate pH 4.5. Crystals were transferred into a cryoprotectant solution containing 40% PEG 300 and 0.1 M Sodium Acetate pH 4.5 and were flash frozen in liquid nitrogen. Diffraction data at 2.8 Å resolution was collected at the Canadian Light Source (CLS-08ID). The diffraction data was indexed, integrated and scaled using XDS³¹ and AIMLESS³². The structure was solved by molecular replacement using Phaser³³ and 1T7D as the search model. Iterative rounds of re-building and refinement using coot³⁴ and Phenix³⁵ resulted in a final model with R/Rfree=0.204/0.252 and good geometry (Table 5).

TABLE 5
Data collection and refinement statistics (molecular
replacement) of G0775-LepB structure

| | G0775 (PDB code) | |
|---|---|---|
| Data collection | | |
| Space group | I 4 | |
| Cell dimensions | | |
| a, b, c (Å) | 109.99 109.99 116.41 | |
| α, β, γ(°) | 90 90 90 | |
| Resolution (Å) | 77.77-2.75 | (2.85-2.75)^{a} |
| R_{merge} | 0.072 | (0.424) |
| I/σ(I) | 8.1 | (1.98) |
| CC_{1/2} | 0.99 | (0.73) |
| Completeness (%) | 96.4 | (100.0) |
| Redundancy | 2.0 | (2.0) |
| Refinement | | |
| Resolution (Å) | 77.77-2.84 | |
| No. reflections | 17393 | (1793) |

| | |
|---|---|
| R_{work}/R_{free} | 0.204/0.252 |
| No. atoms | |
| Protein | 3793 |
| Ligand/ion | 138 |
| Water | 145 |
| B factors | |
| Protein | 57.6 |
| Ligand/ion | 33.77 |
| Water | 41.67 |
| R.m.s. deviations | |
| Bond lengths (Å) | 0.007 |
| Bond angles (°) | 1.48 |

The electron density of G0775 indicates partial occupancy for an unreacted, non-covalently bound form of the inhibitor in each active site in the asymmetric unit (FIG. 8). The appearance of this non-covalent form of the inhibitor is thought to be an artifact that is the result of radiation damage during X-ray data collection since MS analysis indicated that reaction between LepB and G0775 goes to completion.

Enzymology:

A 20 μL reaction in PBS (pH 7.4) containing 0.1% Triton X-100 was initiated by adding 500 pM full length recombinant LepB from E. coli to a 384-well polypropylene assay plate (Thermo Scientific) containing indicated concentration of G0775 and 10 uM fluorogenic peptide substrate Dabcyl-DAP-PAKAAE-Edans (SEQ ID NO: 6) (GL Biochem, Boston, Mass.). Substrate cleavage separates the Dabcyl quencher from the Edans fluorophore, resulting in 490 nm fluorescence (340 nm excitation). The reaction was read kinetically using a pherastar plate reader (BMG Labtech), and curves were fit to a 2-step irreversible binding model using Dynafit software (BioKin Ltd.) to determine Kinact/KI binding constants.

Animal Usage Declaration:

All animal procedures were conducted under protocols approved by the Genentech Institutional Animal Care and Use Committee in an Association for Assessment and Accreditation of Laboratory Animal Care (AAALAC)-accredited facility in accordance with the Guide for the Care and Use of Laboratory Animals and applicable laws and regulations.

Neutropenic Thigh Infection Model.

CD1 mice (Charles River Laboratories) were rendered neutropenic by 2 intraperitoneal injections of Cytoxan (Baxter Health Care Corporation) at 150 mg/kg on Day −5 and 100 mg/kg on Day −2. On day 0, 50 ul log-phase grown bacteria, K. pneumoniae (ATCC 43816) 1×10⁵ CFU/mouse, E. coli (ATCC 25922) 1×10⁵ CFU/mouse, P. aeruginosa (ATCC 27853) 1×10⁶ CFU/mouse, or A. baumannii (ATCC 17978) 1×10⁵ CFU/mouse, were injected into the mouse thigh muscle. At 2 and 11 hours post infection, G0775 was given at indicated dose subcutaneously. At 20 hours post infection, CFU was determined in the thigh muscle through serial dilutions.

Neutropenic Lung Infection Model.

Balb/c mice (Jackson Laboratories) were rendered neutropenic by 2 intraperitoneal injections of Cytoxan (Baxter Health Care Corporation) at 150 mg/kg on Day −4 and 100 mg/kg on Day −1. On day 0, mice were infected intranasally with 40 ul log-phase grown K. pneumoniae (CDC 0106) at 1×10⁶ CFU/mouse. At 2 and 11 hours post infection, G0775 was given at different doses subcutaneously. Ciprofloxacin (Claris Lifesciences Inc.) was dosed at 80 mg/kg subcutaneously once at 2 hour post infection. At 20 hours post infection, CFU was determined in the lung through serial dilutions.

Mucin Peritonitis Survival Study.

CD1 mice (Charles River Laboratories) were infected through intraperitoneal injection of 0.4 mL K. pneumoniae (ATCC 43816) at 3×10⁵ CFU/mouse in 5% mucin (Sigma M1778). At 2 and 11 hours post infection, G0775 was given at the indicated dose subcutaneously. Ciprofloxacin (Claris Lifesciences Inc.) was dosed at 80 mg/kg subcutaneously once at 2 hour post infection. Mice were monitored for body conditioning and survival until 84 hours post infection.

Although the invention has been described with reference to the above examples, it will be understood that modifications and variations are encompassed within the spirit and scope of the invention. Accordingly, the invention is limited only by the following claims.

What is claimed is:

1. An inhibited peptidase, comprising a signal peptidase (SPase) inhibitor having a bond to an amino acid residue of a bacterial type I SPase, a bacterial type I SPase homolog, or a bacterial type I SPase lysine homolog.

2. The inhibited peptidase of claim 1, wherein the inhibitor forms an irreversible bond with the amino acid residue.

3. The inhibited peptidase of claim 1, wherein the bond is a covalent bond.

4. The inhibited peptidase of claim 1, wherein the peptidase comprises region B, region C, region C′, and region D, wherein:
(SEQ ID NO: 1)
B comprises amino acid sequence PSXSMXPTLX;
(SEQ ID NO: 2)
C comprises amino acid sequence DXIXVXKXX;
(SEQ ID NO: 3)
C′ comprises amino acid sequence RGDXXVFXXP;
and
(SEQ ID NO: 4)
D comprises amino acid sequence Y/F, I/V,
KRXXGXXGD,
wherein X is any natural amino acid residue or any unnatural amino acid residue.

5. The inhibited peptidase of claim 4, wherein the inhibitor and the peptidase are bonded at region D of the peptidase.

6. The inhibited peptidase of any one of claims 1-5, wherein the bond is formed between a portion of an electrophilic acceptor moiety on the inhibitor and a portion of the amino acid residue of the peptidase.

7. The inhibited peptidase of claim 6, wherein the electrophilic acceptor moiety on the inhibitor is a —C≡N group.

8. The inhibited peptidase of any one of claims 1-7, wherein the inhibitor forms a bond to a lysine residue of the peptidase.

9. The inhibited peptidase of claim 8, wherein the inhibitor forms a bond to a nitrogen atom of the side chain of the lysine residue.

10. The inhibited peptidase of any one of claims 1-9, wherein the inhibitor is an arylomycin derivative or a bacterial signal peptide, or fragment or homolog thereof.

11. The inhibited peptidase of claim 1, wherein the inhibited peptidase has structural Formula (I): 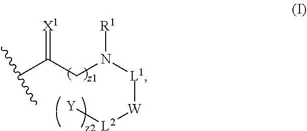
wherein:
X¹ is ═O or ═NH;
n1 is an integer from 0 to 4;
m1 and v1 are independently 1 or 2; z1 is an integer from 1 to 4;
z2 is 0 or 1;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W is a substituted or unsubstituted linear peptide or a substituted or unsubstituted cyclic peptide, wherein the peptide comprises at least three of: natural amino acid residues, unnatural amino acid residues, or a combination thereof;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof; and
R^{1A}, R^{1B}, R^{1C}, and R^{1D} are independently hydrogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl,
wherein “ [symbol] ” indicates the point of attachment between the peptidase and the inhibitor.

12. The inhibited peptidase of claim 1 or 11, wherein the inhibited peptidase has structural Formula (I-A): 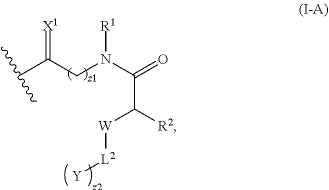
wherein:
X¹ is ═O or ═NH;
n1 is an integer from 0 to 4;
m1 and v1 are independently 1 or 2;
z1 is an integer from 1 to 4;
z2 is 0 or 1;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W is a substituted or unsubstituted linear peptide or a substituted or unsubstituted cyclic peptide, wherein the peptide comprises at least three of: natural amino acid residues, unnatural amino acid residues, or a combination thereof;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof; and
R^{1A}, R^{1B}, R^{1C}, and R^{1D} are independently hydrogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl,
wherein “ [symbol] ” indicates the point of attachment between the peptidase and the inhibitor.

13. The inhibited peptidase of claim 12, wherein X¹ is ═NH.

14. The inhibited peptidase of claim 12, wherein W comprises at least one amino acid selected from 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

15. The inhibited peptidase of claim 12, wherein W comprises glycine and at least one amino acid selected from: 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

16. The inhibited peptidase of claim 12, wherein the inhibited peptidase is

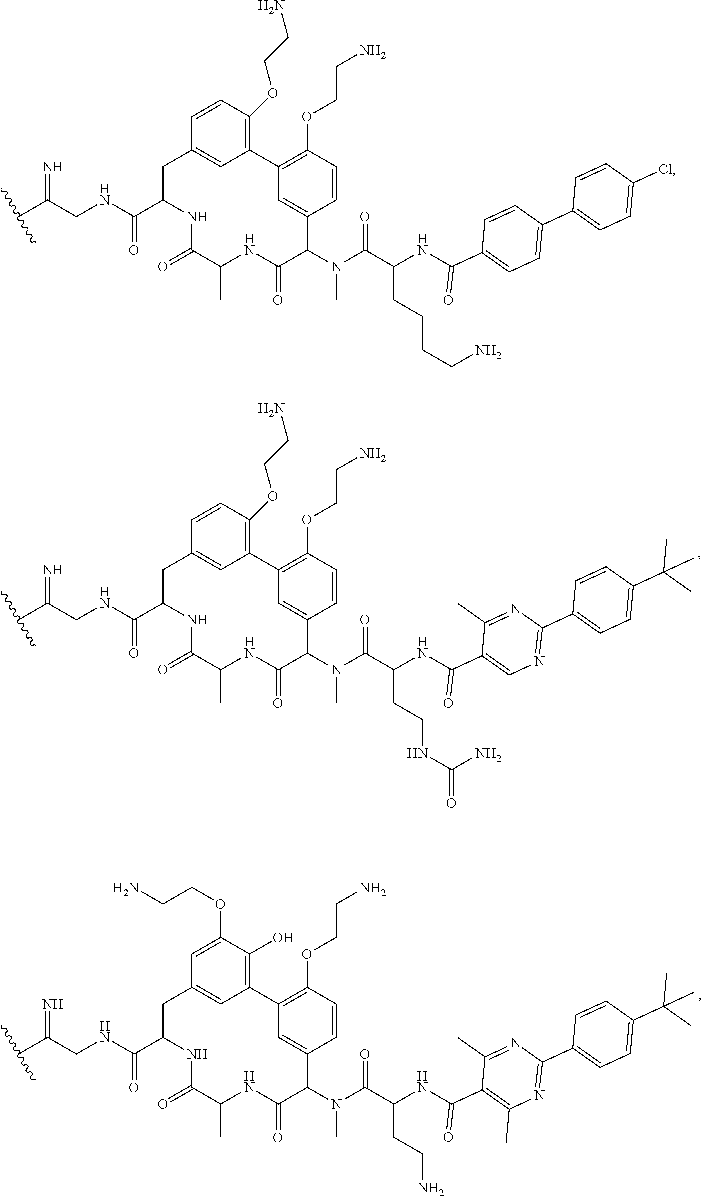 [SMILES: *C(=N)CNC(=O)C1Cc2ccc(OCCN)c(c2)-c2cc(ccc2OCCN)C(N(C)C(=O)C(CCCCN)NC(=O)c2ccc(-c3ccc(Cl)cc3)cc2)C(=O)NC(C)C(=O)N1 ; *C(=N)CNC(=O)C1Cc2ccc(OCCN)c(c2)-c2cc(ccc2OCCN)C(N(C)C(=O)C(CCNC(N)=O)NC(=O)c2cnc(-c3ccc(C(C)(C)C)cc3)nc2C)C(=O)NC(C)C(=O)N1 ; *C(=N)CNC(=O)C1Cc2cc(OCCN)c(O)c(c2)-c2cc(ccc2OCCN)C(N(C)C(=O)C(CCN)NC(=O)c2c(C)nc(-c3ccc(C(C)(C)C)cc3)nc2C)C(=O)NC(C)C(=O)N1]

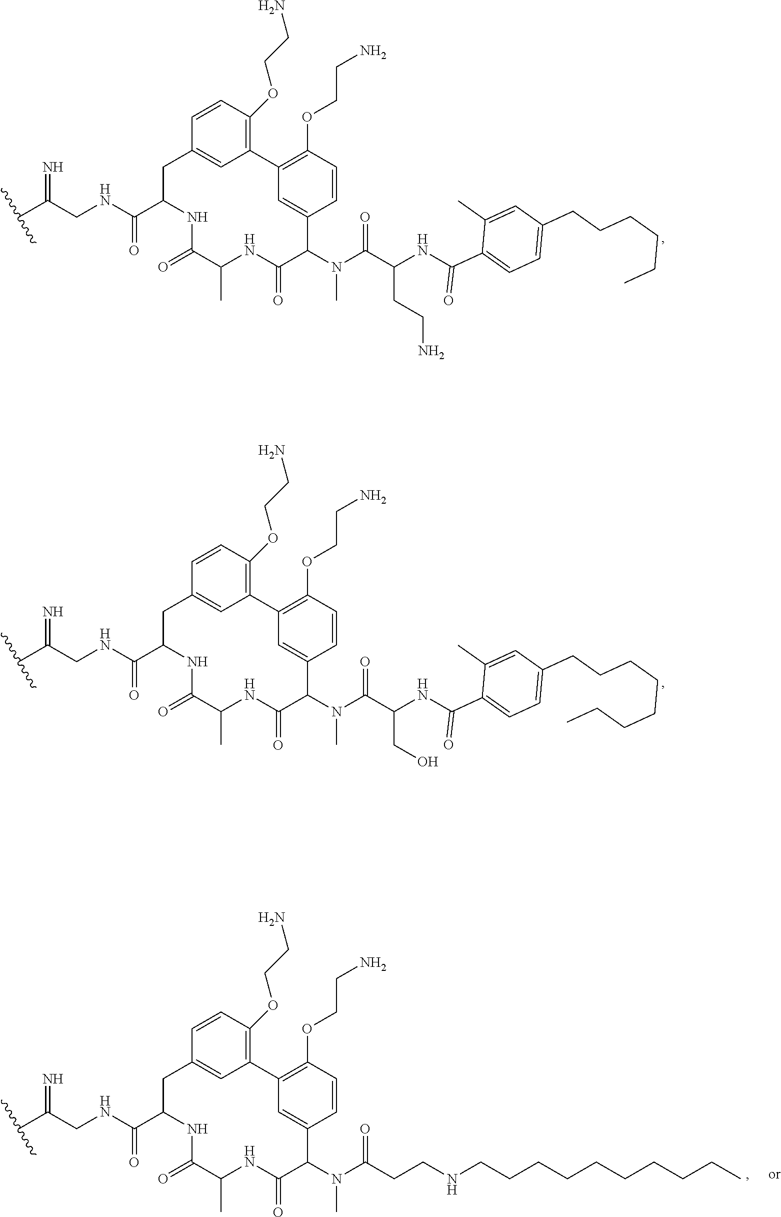 [SMILES: *C(=N)CNC(=O)C1Cc2ccc(OCCN)c(c2)-c2cc(ccc2OCCN)C(N(C)C(=O)C(CCN)NC(=O)c2ccc(CCCCCC)cc2C)C(=O)NC(C)C(=O)N1 ; *C(=N)CNC(=O)C1Cc2ccc(OCCN)c(c2)-c2cc(ccc2OCCN)C(N(C)C(=O)C(CO)NC(=O)c2ccc(CCCCCCCC)cc2C)C(=O)NC(C)C(=O)N1 ; *C(=N)CNC(=O)C1Cc2ccc(OCCN)c(c2)-c2cc(ccc2OCCN)C(N(C)C(=O)CCNCCCCCCCCCC)C(=O)NC(C)C(=O)N1]

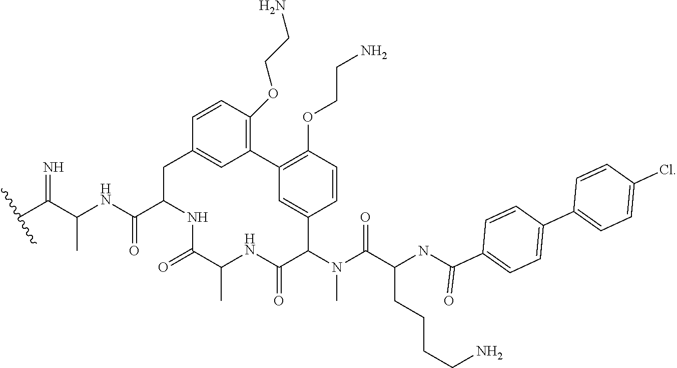 [SMILES: *C(=N)C(C)NC(=O)C1Cc2ccc(OCCN)c(c2)-c2cc(ccc2OCCN)C(N(C)C(=O)C(CCCCN)NC(=O)c2ccc(-c3ccc(Cl)cc3)cc2)C(=O)NC(C)C(=O)N1]

17. The inhibited peptidase of claim 1, wherein the inhibited peptidase has structural Formula (I-B): 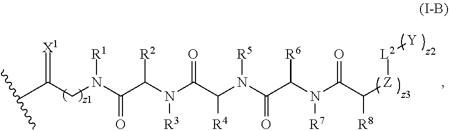
wherein:
X¹ is ═O or ═NH;
n1 is an integer from 0 to 4;
m1 and v1 are independently 1 or 2;
z1 is an integer from 1 to 4;
z2 is 0 or 1;
z3 is an integer from 0 to 20;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R², R⁴, and R⁶ are independently hydrogen,
halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R² and R⁶; or R² and R⁸ may optionally be joined to form a substituted or unsubstituted heterocycloalkyl;
R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl- C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl- C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H) [optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl;
Z is independently a substituted or unsubstituted natural amino acid or a substituted or unsubstituted unnatural amino acid, wherein R² and a Z amino acid; R⁴ and a Z amino acid; R⁶ and a Z amino acid; or two Z amino acids may optionally be joined by a linking moiety, L³, to form a substituted or unsubstituted heterocycloalkyl;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)RD, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof;
R^{1A}, R^{1B}, R^{1C}, and R^{1D} are independently hydrogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl;
R^{8A} is independently hydrogen or —(C₁-C₆)alkyl;
R^{8B} and R^{8C} are independently hydrogen or optionally substituted —(C₁-C₆)alkyl or R^{8B} and R^{8C} and the nitrogen atom to which they are attached optionally form a heterocycloalkyl ring;
R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R⁴)₂, —C(O)N(R⁴)₂, or —SO₂N(R¹⁴)₂; or R^{8D} and R^{8E} and the nitrogen atom to which they are attached optionally form a heterocycloalkyl ring; and
R¹⁴ is independently hydrogen or —(C₁-C₆)alkyl; or two R¹⁴ groups and the nitrogen atom to which they are attached optionally form a heterocycloalkyl ring,
wherein “ [symbol] ” indicates the point of attachment between the peptidase and the inhibitor.

18. The inhibited peptidase of claim 17, wherein R² and R⁶ are joined to form a substituted or unsubstituted heterocycloalkyl.

19. The inhibited peptidase of claim 17, wherein Z comprises at least one amino acid selected from 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

20. The inhibited peptidase of claim 17, wherein Z comprises glycine and at least one amino acid selected from: 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

21. The inhibited peptidase of any one of claims 17-20, wherein z3 is 0.

22. The inhibited peptidase of any one of claim 1 or 17-21, wherein the inhibited peptidase has structural Formula (III): 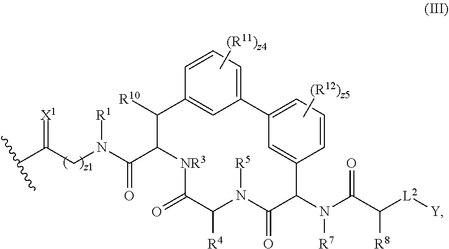
wherein:
X¹ is ═O or ═NH;
n1 is an integer from 0 to 4;
m1 and v1 are independently 1 or 2; z1 is an integer from 1 to 4;
z4 and z5 are independently an integer from 0 to 4;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ and R¹⁰ are independently hydrogen,
halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl- C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl- C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H) [optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl;
R¹¹ is hydrogen,
halogen, —CCl₃, —CBr₃, —CF₃, —CI₃, —CN, —OR^{2A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen,
halogen, —CCl₃, —CBr₃, —CF₃, —CI₃, —CN, —OR^{3A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)RD, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof;
R^{1A}, R^{1B}, R^{1C}, R^{1D}, R^{2A}, and R^{3A} are independently hydrogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl; and
R^{8A} is independently hydrogen or —(C₁-C₆)alkyl;
R^{8B} and R^{8C} are independently hydrogen or optionally substituted —(C₁-C₆)alkyl or R^{8B} and R^{8C} and the nitrogen atom to which they are attached optionally form a heterocycloalkyl ring;
R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R¹⁴)₂, —C(O)N(R¹⁴)₂, or —SO₂N(R¹⁴)₂; or R^{8D} and R^{8E} and the nitrogen atom to which they are attached optionally form a heterocycloalkyl ring; and
R¹⁴ is independently hydrogen or —(C₁-C₆)alkyl; or two R¹⁴ groups and the nitrogen atom to which they are attached optionally form a heterocycloalkyl ring,
wherein “ [symbol] ” indicates the point of attachment between the peptidase and the inhibitor.

23. The inhibited peptidase of claim 22, wherein the inhibited peptidase has structural Formula (III-A): 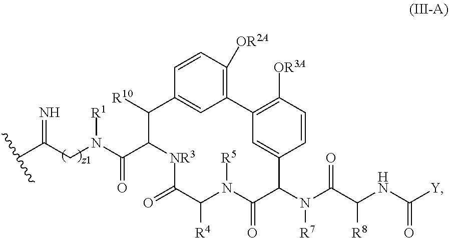
wherein R^{2A} and R^{3A} are independently hydrogen, substituted or unsubstituted alkyl, or substituted or unsubstituted heteroalkyl.

24. The inhibited peptidase of claim 23, wherein Y is substituted or unsubstituted aryl or substituted or unsubstituted heteroaryl.

25. The inhibited peptidase of claim 24, wherein the inhibited peptidase has structural Formula (III-B): 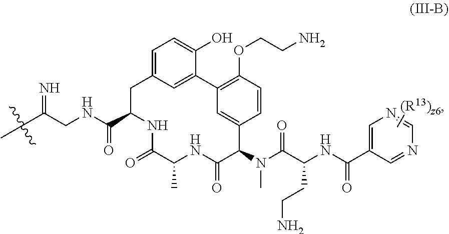
wherein:
z6 is an integer from 0 to 3; and
R¹³ is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHC₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

26. The inhibited peptidase of claim 24, wherein R^{2A} and R^{3A} are independently substituted or unsubstituted heteroalkyl.

27. The inhibited peptidase of claim 26, wherein the inhibited peptidase has structural Formula (III-C): 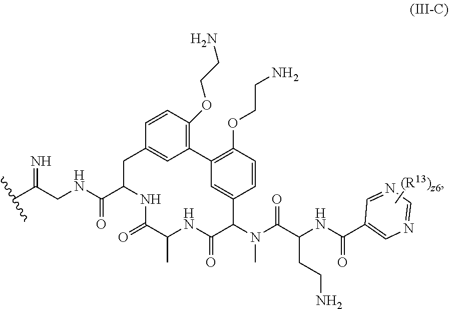
wherein:
z6 is an integer from 0 to 3; and
R¹³ is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

28. The inhibited peptidase of claim 27, wherein R¹³ is independently substituted or unsubstituted alkyl, substituted or unsubstituted aryl, or substituted or unsubstituted heteroaryl.

29. The inhibited peptidase of claim 28, wherein the inhibited peptidase has structural Formula (III-D):

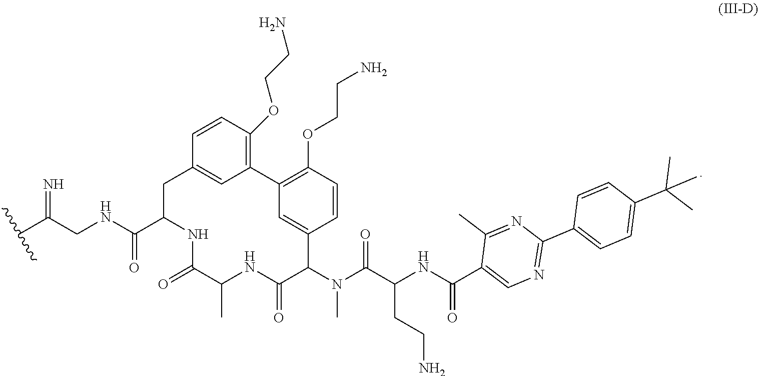 [SMILES: *C(=N)CNC(=O)C1Cc2ccc(OCCN)c(c2)-c2cc(ccc2OCCN)C(N(C)C(=O)C(CCN)NC(=O)c2cnc(-c3ccc(C(C)(C)C)cc3)nc2C)C(=O)NC(C)C(=O)N1]

30. The inhibited peptidase of any one of claims 11-29, wherein the inhibitor forms an irreversible bond with the amino acid residue.

31. The inhibited peptidase of any one of claims 11-30, wherein the bond is a covalent bond.

32. The inhibited peptidase of any one of claims 11-31, wherein the peptidase comprises region B, region C, region C′, and region D, wherein:
(SEQ ID NO: 1)
B comprises amino acid sequence PSXSMXPTLX;
(SEQ ID NO: 2)
C comprises amino acid sequence DXIXVXKXX;
(SEQ ID NO: 3)
C′ comprises amino acid sequence RGDXXVFXXP; and
(SEQ ID NO: 4)
D comprises amino acid sequence Y/F, I/V,
KRXXGXXGD,
wherein X is any natural amino acid residue or any unnatural amino acid residue.

33. The inhibited peptidase of claim 32, wherein the inhibitor and the peptidase are bonded at region D of the peptidase.

34. The inhibited peptidase of any one of claims 11-33, wherein the bond is formed between a portion of an electrophilic acceptor moiety on the inhibitor and a portion of the amino acid residue of the peptidase.

35. The inhibited peptidase of claim 34, wherein the electrophilic acceptor moiety on the inhibitor is a —C≡N group.

36. The inhibited peptidase of any one of claims any one of claims 11-35, wherein the inhibitor forms a bond to a lysine residue of the peptidase.

37. The inhibited peptidase of claim 36, wherein the inhibitor forms a bond to a nitrogen atom of the side chain of the lysine residue.

38. The inhibited peptidase of any one of claims 1-37, wherein the inhibited peptidase is in a bacterial cell.

39. The inhibited peptidase of any one of claims 1-38, wherein the amino acid is Lys¹⁴⁶ of E. coli.

40. The inhibited peptidase of any one of claims 1-3 or 11, wherein the signal peptidase is a Gram-positive a signal peptidase or a Gram-negative a signal peptidase.

41. The inhibited peptidase of any one of claims 1-40, wherein the signal peptidase is a Gram-negative a signal peptidase.

42. The inhibited peptidase of any one of claims 1-41, wherein the signal peptidase is LepB.

43. A method of inhibiting a bacterial peptidase, comprising contacting a bacterial cell with a compound of structural Formula (IV): 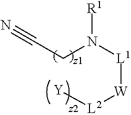
(IV), or a pharmaceutically acceptable salt thereof,
wherein:
n1 is an integer from 0 to 4;
m1 and v1 are independently 1 or 2;
z1 is an integer from 1 to 4;
z2 is 0 or 1;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W is a substituted or unsubstituted linear peptide or a substituted or unsubstituted cyclic peptide, wherein the peptide comprises at least three of: natural amino acid residues, unnatural amino acid residues, or a combination thereof;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof; and
R^{1A}, R^{1B}, R^{1C}, and R^{1D} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl.

44. The method of claim 43, wherein W comprises at least one amino acid selected from 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

45. The method of claim 43, wherein W comprises glycine and at least one amino acid selected from: 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

46. The method of claim 43, wherein the compound is

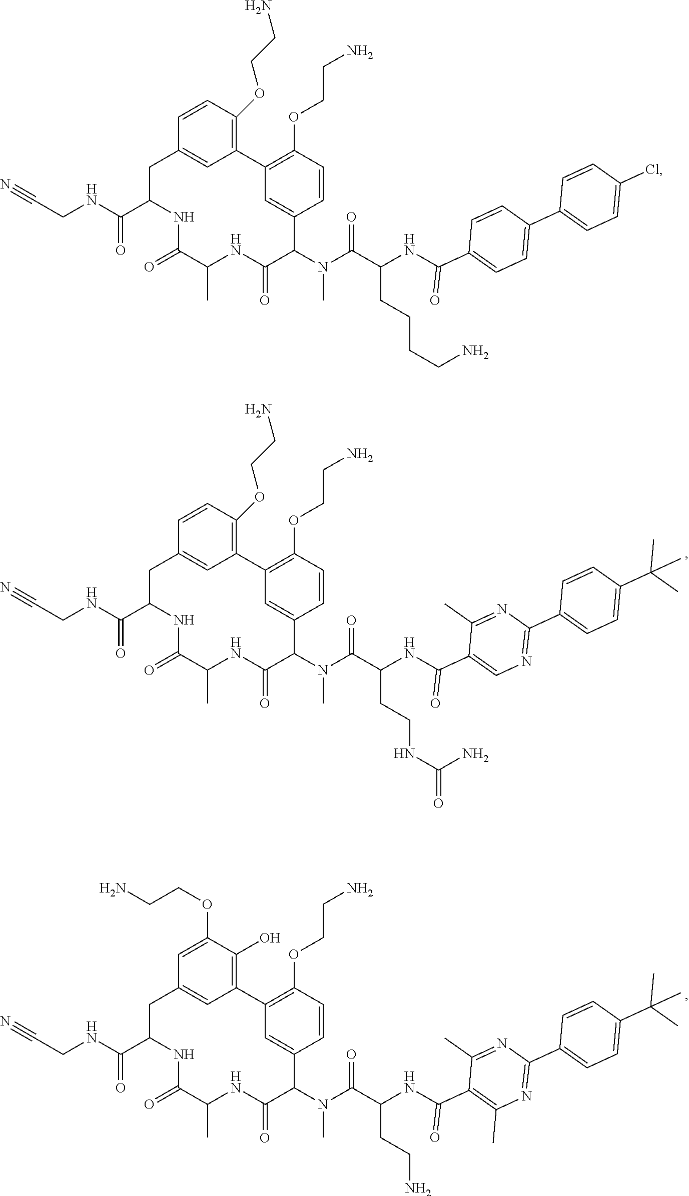 [SMILES: CC1NC(=O)C(N(C)C(=O)C(CCCCN)NC(=O)c2ccc(-c3ccc(Cl)cc3)cc2)c2ccc(OCCN)c(c2)-c2cc(ccc2OCCN)CC(C(=O)NCC#N)NC1=O ; Cc1nc(-c2ccc(C(C)(C)C)cc2)ncc1C(=O)NC(CCNC(N)=O)C(=O)N(C)C1C(=O)NC(C)C(=O)NC(C(=O)NCC#N)Cc2ccc(OCCN)c(c2)-c2cc1ccc2OCCN ; Cc1nc(-c2ccc(C(C)(C)C)cc2)nc(C)c1C(=O)NC(CCN)C(=O)N(C)C1C(=O)NC(C)C(=O)NC(C(=O)NCC#N)Cc2cc(OCCN)c(O)c(c2)-c2cc1ccc2OCCN]

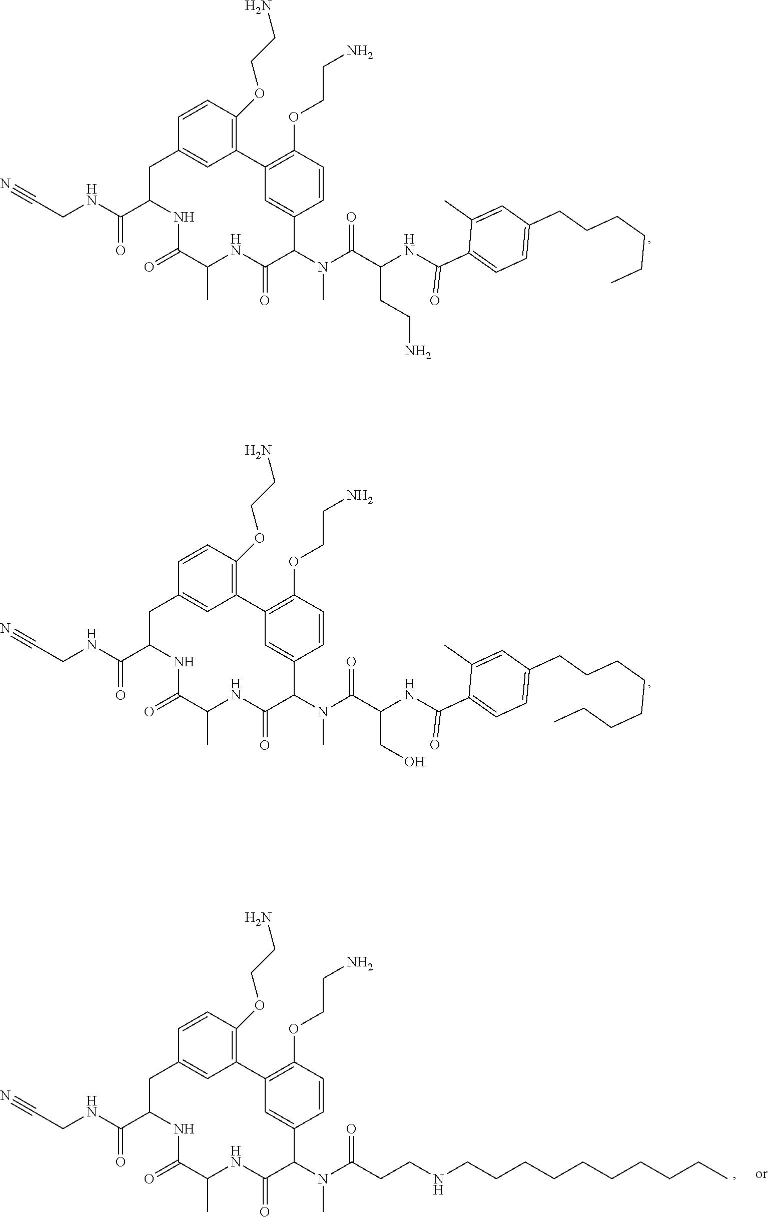 [SMILES: CCCCCCc1ccc(C(=O)NC(CCN)C(=O)N(C)C2C(=O)NC(C)C(=O)NC(C(=O)NCC#N)Cc3ccc(OCCN)c(c3)-c3cc2ccc3OCCN)c(C)c1 ; CCCCCCCCc1ccc(C(=O)NC(CO)C(=O)N(C)C2C(=O)NC(C)C(=O)NC(C(=O)NCC#N)Cc3ccc(OCCN)c(c3)-c3cc2ccc3OCCN)c(C)c1 ; CCCCCCCCCCNCCC(=O)N(C)C1C(=O)NC(C)C(=O)NC(C(=O)NCC#N)Cc2ccc(OCCN)c(c2)-c2cc1ccc2OCCN]

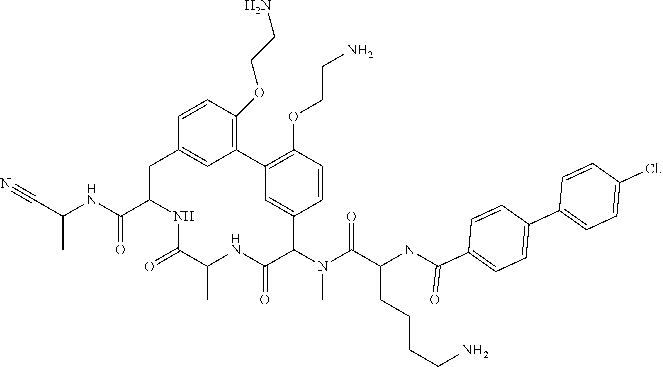 [SMILES: CC(C#N)NC(=O)C1Cc2ccc(OCCN)c(c2)-c2cc(ccc2OCCN)C(N(C)C(=O)C(CCCCN)NC(=O)c2ccc(-c3ccc(Cl)cc3)cc2)C(=O)NC(C)C(=O)N1]

47. The method of claim 43, wherein the compound has structural Formula (IV-C): 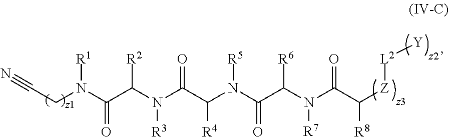
or a pharmaceutically acceptable salt thereof,
wherein:
z3 is an integer from 0 to 20;
R², R⁴, and R⁶ are independently hydrogen,
halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R² and R⁶; or R² and R⁸ may optionally be joined to form a substituted or unsubstituted heterocycloalkyl;
R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, —NH₂, —(C₁-C₆)alkyl, —(C₁-C₆)alkyl-OR^{A}, —(C₁-C₆)alkyl-SR^{A}, —(C₁-C₆)alkyl- C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl- C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H) [optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl;
Z is independently a substituted or unsubstituted natural amino acid or a substituted or unsubstituted unnatural amino acid, wherein R² and a Z amino acid; R⁴ and a Z amino acid; R⁶ and a Z amino acid; or two Z amino acids may optionally be joined by a linking moiety, L³, to form a substituted or unsubstituted heterocycloalkyl;
L³ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{8A} is independently hydrogen or —(C₁-C₆)alkyl;
R^{8B} and R^{8C} are independently hydrogen or optionally substituted —(C₁-C₆)alkyl or R^{8B} and R^{8C} and the nitrogen atom to which they are attached optionally form a heterocycloalkyl ring;
R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R¹⁴)₂, —C(O)N(R¹⁴)₂, or —SO₂N(R¹⁴)₂; or R^{8D} and R^{8E} and the nitrogen atom to which they are attached optionally form a heterocycloalkyl ring; and
R¹⁴ is independently hydrogen or —(C₁-C₆)alkyl; or two R¹⁴ groups and the nitrogen atom to which they are attached optionally form a heterocycloalkyl ring.

48. The method of claim 47, wherein Z comprises at least one amino acid selected from 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

49. The method peptidase of claim 47, wherein Z comprises glycine and at least one amino acid selected from: 2,4-diaminobutanoic acid, 2,5-diaminopentanoic acid, 2,6-diaminohexanoic acid, 2-amino-4-hydroxybutanoic acid, 2-amino-5-hydroxypentanoic acid, and 2-amino-6-hydroxyhexanoic acid.

50. The method of claim 47, wherein R² and R⁶ are joined to form a substituted or unsubstituted heterocycloalkyl.

51. The method of claim 49 or 50, wherein z3 is 0.

52. The method of any one of claims 43-47, wherein the compound has structural Formula (V): 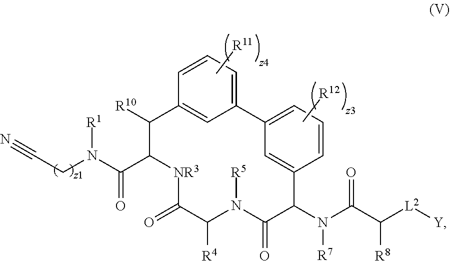
or a pharmaceutically acceptable salt thereof,
wherein:
z4 and z5 are independently an integer from 0 to 4;
R⁴ and R¹⁰ are independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R² and R⁶; or R² and R⁸ may optionally be joined to form a substituted or unsubstituted heterocycloalkyl;
R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl- C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl- C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H) [optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl;
R¹¹ is hydrogen,
halogen, —CCl₃, —CBr₃, —CF₃, —CI₃, —CN, —OR^{2A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen,
halogen, —CCl₃, —CBr₃, —CF₃, —CI₃, —CN, —OR^{3A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A} and R^{3A} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl;
R^{8A} is independently hydrogen or —(C₁-C₆)alkyl;
R^{8B} and R^{8C} are independently hydrogen or optionally substituted —(C₁-C₆)alkyl or R^{8B} and R^{8C} and the nitrogen atom to which they are attached optionally form a heterocycloalkyl ring;
R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R⁴)₂, —C(O)N(R⁴)₂, or —SO₂N(R⁴)₂; or R^{8D} and R^{8E} and the nitrogen atom to which they are attached optionally form a heterocycloalkyl ring; and
R¹⁴ is independently hydrogen or —(C₁-C₆)alkyl; or two R¹⁴ groups and the nitrogen atom to which they are attached optionally form a heterocycloalkyl ring.

53. The method of claim 52, wherein the compound has structural Formula (V-A): 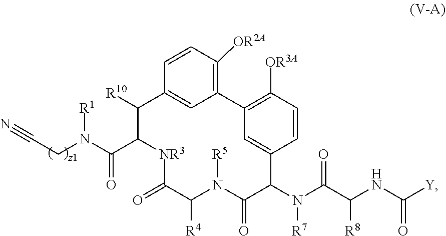
or a pharmaceutically acceptable salt thereof,
wherein R^{2A} and R^{3A} are independently hydrogen, substituted or unsubstituted alkyl, or substituted or unsubstituted heteroalkyl.

54. The method of claim 53, wherein Y is substituted or unsubstituted aryl or substituted or unsubstituted heteroaryl.

55. The method of claim 54, wherein R^{2A} and R^{3A} are independently substituted or unsubstituted heteroalkyl.

56. The method of claim 55, wherein the compound has structural Formula (V-B): 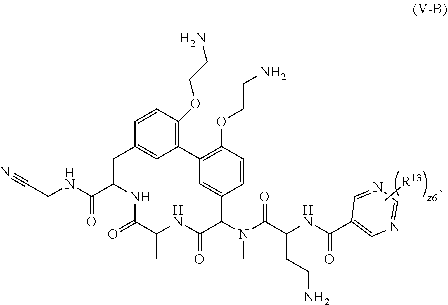
or a pharmaceutically acceptable salt thereof,
wherein:
z6 is an integer from 0 to 3; and
R¹³ is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

57. The method of claim 56, wherein R¹³ is independently substituted or unsubstituted alkyl, substituted or unsubstituted aryl, or substituted or unsubstituted heteroaryl.

58. The method of claim 57, wherein the compound has structural Formula (V-C): 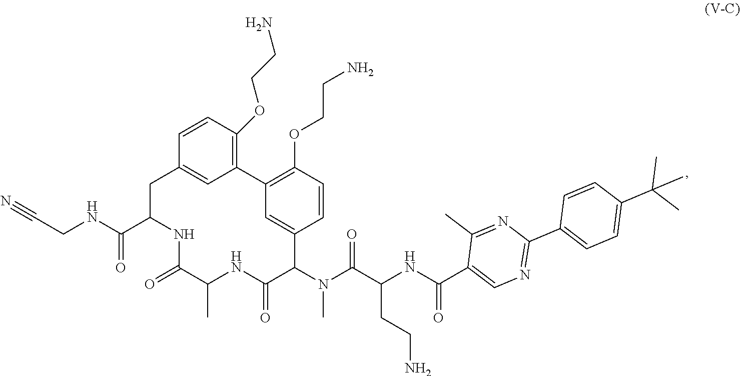 [SMILES: Cc1nc(-c2ccc(C(C)(C)C)cc2)ncc1C(=O)NC(CCN)C(=O)N(C)C1C(=O)NC(C)C(=O)NC(C(=O)NCC#N)Cc2ccc(OCCN)c(c2)-c2cc1ccc2OCCN]
or a pharmaceutically acceptable salt thereof.

59. The method of any one of claims 43-58, wherein the bacterial peptidase is a signal peptidase (SPase).

60. The method of claim 59, wherein the SPase is a bacterial type I SPase.

61. The method of any one of claims 43-58, wherein the compound forms an irreversible bond with the bacterial peptidase.

62. The method of any one of claims 43-58, wherein the compound forms a covalent bond with the bacterial peptidase.

63. The method of any one of claims 43-58, wherein the bacterial the peptidase comprises region B, region C, region C′, and region D, wherein:
B comprises amino acid sequence
(SEQ ID NO: 1)
PSXSMXPTLX;
C comprises amino acid sequence
(SEQ ID NO: 2)
DXIXVXKXX;
C′ comprises amino acid sequence
(SEQ ID NO: 3)
RGDXXVFXXP;
and
D comprises amino acid sequence Y/F, I/V,
(SEQ ID NO: 4)
KRXXGXXGD,
wherein X is any natural amino acid residue or any unnatural amino acid residue.

64. The method of claim 63, wherein the bacterial peptidase and the compound bond at region D of the peptidase.

65. The method of any one of claims 43-64, wherein the compound forms a bond with a lysine residue of the bacterial peptidase.

66. The method of claim 65, wherein the bond is formed at the side chain of the lysine residue.

67. The inhibited peptidase of claim 66, wherein the bacterial peptidase is LepB.

68. The inhibited peptidase of claim 67, wherein the lysine residue is Lys¹⁴⁶.

69. The method of any one of claims 43-66, wherein the bacterial peptidase is selected from E. coli, L. monocytogenes, M. leprae, M. tuberculosis, M. ulcerans, M. pneumoniae, K. pneumoniae, K. pneumoniae, E. aerogenes, C. werkmanii, S. marcescens, S. marcescens, A. baumannii, N. gonorrhoeae, and N. meningitides.

70. A method of inhibiting signal protein secretion of a bacterial cell, comprising contacting the cell with a compound of structural Formula (IV): 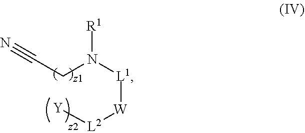
or a pharmaceutically acceptable salt thereof,
wherein:
n1 is an integer from 0 to 4;
m1 and v1 are independently 1 or 2;
z1 is an integer from 1 to 4;
z2 is 0 or 1;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W is a substituted or unsubstituted linear peptide or a substituted or unsubstituted cyclic peptide, wherein the peptide comprises at least three of: natural amino acid residues, unnatural amino acid residues, or a combination thereof;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof; and
R^{1A}, R^{1B}, R^{1C}, and R^{1D} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl.

71. A method of treating a bacterial infection, comprising administering to a subject in need thereof a therapeutically effective amount of a compound of structural Formula (IV): 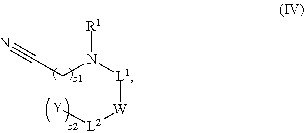
or a pharmaceutically acceptable salt thereof,
wherein:
n1 is an integer from 0 to 4;
m1 and v1 are independently 1 or 2;
z1 is an integer from 1 to 4;
z2 is 0 or 1;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W is a substituted or unsubstituted linear peptide or a substituted or unsubstituted cyclic peptide, wherein the peptide comprises at least three of: natural amino acid residues, unnatural amino acid residues, or a combination thereof;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is-SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof; and
R^{1A}, R^{1B}, R^{1C}, and R^{1D} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl.

72. The method of claim 71, wherein the compound has structural Formula (IV-C): 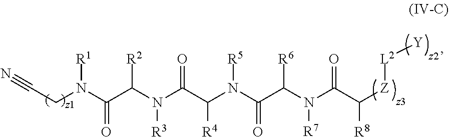
or a pharmaceutically acceptable salt thereof,
wherein:
z3 is an integer from 0 to 20;
R², R⁴, and R⁶ are independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R² and R⁶; or R² and R⁸ may optionally be joined to form a substituted or unsubstituted heterocycloalkyl;
R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl- C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl- C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H)[optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl;
Z is independently a substituted or unsubstituted natural amino acid or a substituted or unsubstituted unnatural amino acid, wherein R² and a Z amino acid; R⁴ and a Z amino acid; R⁶ and a Z amino acid; or two Z amino acids may optionally be joined by a linking moiety, L³, to form a substituted or unsubstituted heterocycloalkyl; and
L³ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{8A} is independently hydrogen or —(C₁-C₆)alkyl;
R^{8B} and R^{8C} are independently hydrogen or optionally substituted —(C₁-C₆)alkyl or R^{8B} and R^{8C} and the nitrogen atom to which they are attached optionally form a heterocycloalkyl ring;
R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R¹⁴)₂, —C(O)N(R¹⁴)₂, or —SO₂N(R¹⁴)₂; or R^{8D} and R^{8E} and the nitrogen atom to which they are attached optionally form a heterocycloalkyl ring; and
R¹⁴ is independently hydrogen or —(C₁-C₆)alkyl; or two R¹⁴ groups and the nitrogen atom to which they are attached optionally form a heterocycloalkyl ring.

73. The method of claim 72, wherein R² and R⁶ are joined to form a substituted or unsubstituted heterocycloalkyl.

74. The method of claim 72 or 73, wherein z3 is 0.

75. The method of any one of claims 71-74, wherein the compound has structural Formula (V): 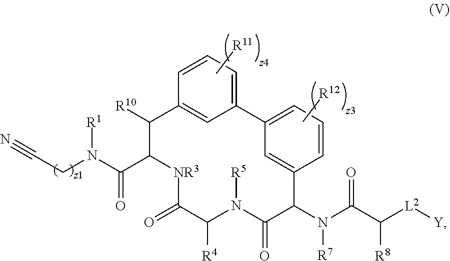
or a pharmaceutically acceptable salt thereof,
wherein:
z4 and z5 are independently an integer from 0 to 4;
R⁴ and R¹⁰ are independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R² and R⁶; or R² and R⁸ may optionally be joined to form a substituted or unsubstituted heterocycloalkyl;
R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl- C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl- C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H) [optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl;
R¹¹ is hydrogen,
halogen, —CCl₃, —CBr₃, —CF₃, —Cl₃, —CN, —OR^{2A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen,
halogen, —CCl₃, —CBr₃, —CF₃, —Cl₃, —CN, —OR^{3A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A} and R^{3A} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl;
R^{8A} is independently hydrogen or —(C₁-C₆)alkyl;
R^{8B} and R^{8C} are independently hydrogen or optionally substituted —(C₁-C₆)alkyl or R^{8B} and R^{8C} and the nitrogen atom to which they are attached optionally form a heterocycloalkyl ring;
R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R¹⁴)₂, —C(O)N(R¹⁴)₂, or —SO₂N(R¹⁴)₂; or R^{8D} and R^{8E} and the nitrogen atom to which they are attached optionally form a heterocycloalkyl ring; and
R¹⁴ is independently hydrogen or —(C₁-C₆)alkyl; or two R¹⁴ groups and the nitrogen atom to which they are attached optionally form a heterocycloalkyl ring.

76. The method of claim 75, wherein the compound has structural Formula (V-A): 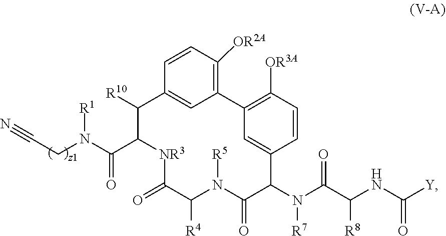
or a pharmaceutically acceptable salt thereof,
wherein R^{2A} and R^{3A} are independently hydrogen, substituted or unsubstituted alkyl, or substituted or unsubstituted heteroalkyl.

77. The method of claim 76, wherein Y is substituted or unsubstituted aryl or substituted or unsubstituted heteroaryl.

78. The method of claim 77, wherein R^{2A} and R^{3A} are independently substituted or unsubstituted heteroalkyl.

79. The method of claim 78, wherein the compound has structural Formula (V-B): 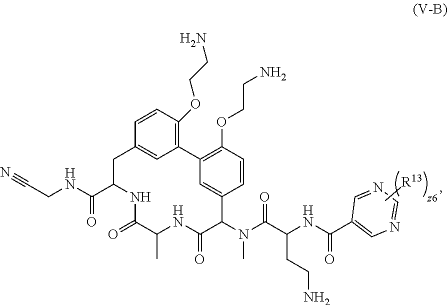
or a pharmaceutically acceptable salt thereof,
wherein:
z6 is an integer from 0 to 3; and
R¹³ is independently hydrogen,
halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

80. The method of claim 70, wherein R¹³ is independently substituted or unsubstituted alkyl, substituted or unsubstituted aryl, or substituted or unsubstituted heteroaryl.

81. The method of claim 71, wherein the compound has structural Formula (V-D): 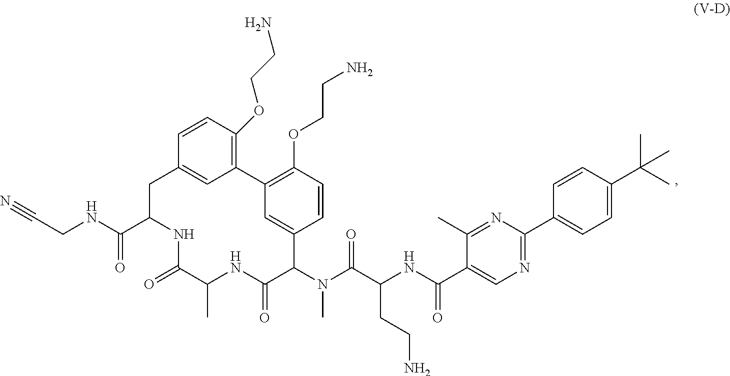 [SMILES: Cc1nc(-c2ccc(C(C)(C)C)cc2)ncc1C(=O)NC(CCN)C(=O)N(C)C1C(=O)NC(C)C(=O)NC(C(=O)NCC#N)Cc2ccc(OCCN)c(c2)-c2cc1ccc2OCCN]
or a pharmaceutically acceptable salt thereof.

82. The method of any one of claims 71-81, wherein the bacterial infection is caused by E. coli, L. monocytogenes, M. leprae, M. tuberculosis, M. ulcerans, M. pneumoniae, K. pneumoniae, K. pneumoniae, E. aerogenes, C. werkmanii, S. marcescens, S. marcescens, A. baumannii, N. gonorrhoeae, or N. meningitides.

83. The method of any one of claims 71-81, wherein the bacterial infection is caused by methicillin-resistant (MRSA) bacteria, vancomycin-intermediate (VISA) bacteria, vancomycin-resistant Staphylococcus aureus (VRSA), multidrug-resistant (MDR) bacteria, pandrug-resistant (PDR) Gram-negative bacteria, or extensively drug-resistant (XDR) bacteria.

84. The method of claim 83, wherein the PDR Gram-negative bacteria is Pseudomonas aeruginosa, Acinetobacter baumannii, or Klebsiella pneumoniae.

85. The method of claim 83, wherein the PDR and XDR bacteria are independently Mycobacterium tuberculosis.

86. The method of any one of claims 71-81, wherein the bacterial infection is caused by a Gram-negative bacteria.

87. The method of any one of claims 71-86, wherein the bacterial infection is caused an indwelling device or a prosthetic device.

88. The method of any one of claims 71-87, wherein the bacterial infection is caused by a biofilm-associated bacteria.

89. The method of any one of claims 71-88, further comprising administering to the subject an antibiotic in combination with the compound of structural Formula (IV).

90. The method of claim 89, wherein the antibiotic is an aminoglycoside, a fluoroquinolone, a carbapenem, a tetracycline, or an arylomycin.

91. The method of claim 89, wherein the antibiotic is ceftazidime, avibactam, levofloxacin, meropenem, colistin, or tigecycline.

92. An inhibited peptide, comprising:
a serine-lysine catalytic dyad or a serine-serine lysine catalytic triad; and
a peptide inhibitor having a bond to an amino group of the lysine.

93. The inhibited peptide of claim 92, wherein the inhibited peptide is a bacterial peptide or a mammalian peptide.

94. The inhibited peptide of claim 92 or 93, wherein the inhibited peptide is selected from bacterial UmuD, bacterial LexA, bacterial Lon protease, bacterial signal peptidase, bacterial penicillin binding protein V, bacterial penicillin binding protein 1a, bacterial penicillin binding protein 1b, bacterial penicillin binding protein 2; bacterial penicillin binding protein 3; mammalian lactoferrin; mammalian mitochondrial signal peptidase; N-terminal Serine or Threonine protease; bacterial penicillin G acylase precursor; mammalian glycosylasparaginase precursor, and a bacterial penicillin binding proteins homologous to E. coli PBP1a, 1b, 2, 3, 4, 5, or 6.

95. The inhibited peptide of claim 92 or 93, wherein the inhibited bacterial peptide is selected from Escherichia coli UmuD, Escherichia coli LexA, Escherichia coli Lon protease, Escherichia coli signal peptidase, Escherichia coli penicillin binding protein V; Escherichia coli penicillin binding protein 1a, Escherichia coli penicillin binding protein 1b, Escherichia coli penicillin binding protein 2, Escherichia coli penicillin binding protein 3, Homo sapiens lactoferrin, Homo sapiens mitochondrial signal peptidase, N-terminal Serine or Threonine protease, Escherichia coli penicillin G acylase precursor, Homo sapiens glycosylasparaginase precursor, and an Escherichia coli penicillin binding proteins homologous to E. coli PBP1a, 1b, 2, 3, 4, 5, or 6.

96. The inhibited peptide of any one of claims 92-95, wherein the inhibited peptide has structural Formula (I): 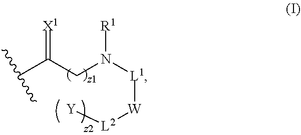
wherein:
X¹ is ═O or ═NH;
n1 is an integer from 0 to 4;
m1 and v1 are independently 1 or 2;
z1 is an integer from 1 to 4;
z2 is 0 or 1;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W is a substituted or unsubstituted linear peptide or a substituted or unsubstituted cyclic peptide, wherein the peptide comprises at least three of: natural amino acid residues, unnatural amino acid residues, or a combination thereof;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof; and
R^{1A}, R^{1B}, R^{1C}, and R^{1D} are independently hydrogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl,
wherein “ [symbol] ” indicates the point of attachment between the peptide and the inhibitor.

97. The inhibited peptide of claim 92 or 96, wherein the inhibited peptide has structural Formula (I-A): 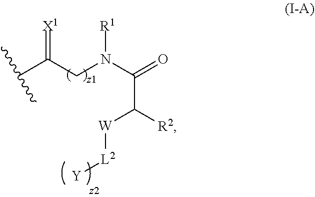
wherein:
X¹ is ═O or ═NH;
n1 is an integer from 0 to 4;
m1 and v1 are independently 1 or 2;
z1 is an integer from 1 to 4;
z2 is 0 or 1;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W is a substituted or unsubstituted linear peptide or a substituted or unsubstituted cyclic peptide, wherein the peptide comprises at least three of: natural amino acid residues, unnatural amino acid residues, or a combination thereof;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof; and
R^{1A}, R^{1B}, R^{1C}, and R^{1D} are independently hydrogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl,
wherein “ [symbol] ” indicates the point of attachment between the peptide and the inhibitor.

98. The inhibited peptide of claim 97, wherein X¹ is ═NH.

99. The inhibited peptide of claim 92, wherein the inhibited peptide has structural Formula (I-B): 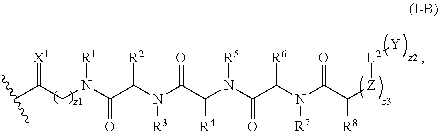
wherein:
X¹ is ═O or ═NH;
n1 is an integer from 0 to 4;
m1 and v1 are independently 1 or 2;
z1 is an integer from 1 to 4;
z2 is 0 or 1;
z3 is an integer from 0 to 20;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R², R⁴, and R⁶ are independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHC₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R² and R⁶; or R² and R⁸ may optionally be joined to form a substituted or unsubstituted heterocycloalkyl;
R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl- C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl- C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H)[optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl;
Z is independently a substituted or unsubstituted natural amino acid or a substituted or unsubstituted unnatural amino acid, wherein R² and a Z amino acid; R⁴ and a Z amino acid; R⁶ and a Z amino acid; or two Z amino acids may optionally be joined by a linking moiety, L³, to form a substituted or unsubstituted heterocycloalkyl;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)RD, —C(O)OR^{1D}, —C(O)NR^{1B}R^{1C}, —OR^{1A}, —NR^{1B}SO₂R^{1A}, NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof;
R^{1A}, R^{1B}, R^{1C}, and R^{1D} are independently hydrogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl;
R^{8A} is independently hydrogen or —(C₁-C₆)alkyl;
R^{8B} and R^{8C} are independently hydrogen or optionally substituted —(C₁-C₆)alkyl or R^{8B} and R^{8C} and the nitrogen atom to which they are attached optionally form a heterocycloalkyl ring;
R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R⁴)₂, —C(O)N(R⁴)₂, or —SO₂N(R⁴)₂; or R^{8D} and R^{8E} and the nitrogen atom to which they are attached optionally form a heterocycloalkyl ring; and
R¹⁴ is independently hydrogen or —(C₁-C₆)alkyl; or two R¹⁴ groups and the nitrogen atom to which they are attached optionally form a heterocycloalkyl ring,
wherein “ [symbol] ” indicates the point of attachment between the peptide and the inhibitor.

100. The inhibited peptide of claim 99, wherein R² and R⁶ are joined to form a substituted or unsubstituted heterocycloalkyl.

101. The inhibited peptide of claim 99 or 100, wherein z3 is 0.

102. The inhibited peptide of any one of claim 92 or 100-101, wherein the inhibited peptide has structural Formula (III): 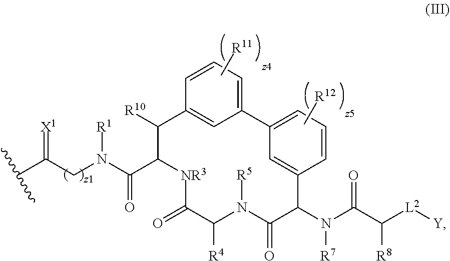
wherein:
X¹ is ═O or ═NH;
n1 is an integer from 0 to 4;
m1 and v1 are independently 1 or 2;
z1 is an integer from 1 to 4;
z4 and z5 are independently an integer from 0 to 4;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ and R¹⁰ are independently hydrogen,
halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl- C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl- C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H)[optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl;
R¹¹ is hydrogen,
halogen, —CCl₃, —CBr₃, —CF₃, —CI₃, —CN, —OR^{2A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen,
halogen, —CCl₃, —CBr₃, —CF₃, —CI₃, —CN, —OR^{3A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)RD, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{1D}, —NR^{1B}OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof;
R^{1A}, R^{1B}, R^{1C}, R^{1D}, R^{2A}, and R^{3A} are independently hydrogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl; and
R^{8A} is independently hydrogen or —(C₁-C₆)alkyl;
R^{8B} and R^{8C} are independently hydrogen or optionally substituted —(C₁-C₆)alkyl or R^{8B} and R^{8C} and the nitrogen atom to which they are attached optionally form a heterocycloalkyl ring;
R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R⁴)₂, —C(O)N(R⁴)₂, or —SO₂N(R⁴)₂; or R^{8D} and R^{8E} and the nitrogen atom to which they are attached optionally form a heterocycloalkyl ring; and
R¹⁴ is independently hydrogen or —(C₁-C₆)alkyl; or two R¹⁴ groups and the nitrogen atom to which they are attached optionally form a heterocycloalkyl ring,
wherein “ [symbol] ” indicates the point of attachment between the peptide and the inhibitor.

103. The inhibited peptide of claim 102, wherein the inhibited peptide has structural Formula (III-A): 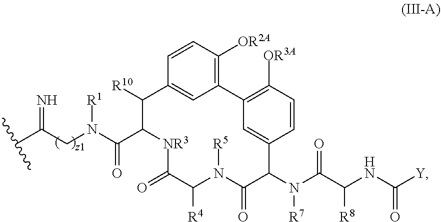
wherein R^{2A} and R^{3A} are independently hydrogen, substituted or unsubstituted alkyl, or substituted or unsubstituted heteroalkyl.

104. The inhibited peptidase of claim 103, wherein Y is substituted or unsubstituted aryl or substituted or unsubstituted heteroaryl.

105. The inhibited peptide of claim 104, wherein the inhibited peptide has structural Formula (Ill-B): 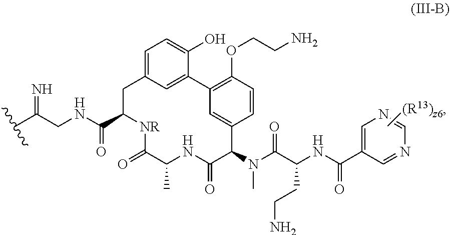
wherein:
z6 is an integer from 0 to 3; and
R¹³ is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

106. The inhibited peptide of claim 104, wherein R^{2A} and R^{3A} are independently substituted or unsubstituted heteroalkyl.

107. The inhibited peptide of claim 106, wherein the inhibited peptide has structural Formula (III-C): 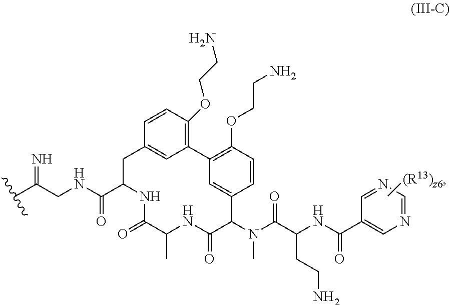
wherein:
z6 is an integer from 0 to 3; and
R¹³ is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

108. The inhibited peptide of claim 107, wherein R¹³ is independently substituted or unsubstituted alkyl, substituted or unsubstituted aryl, or substituted or unsubstituted heteroaryl.

109. The inhibited peptide of claim 108, wherein the inhibited peptide has structural Formula (III-D):

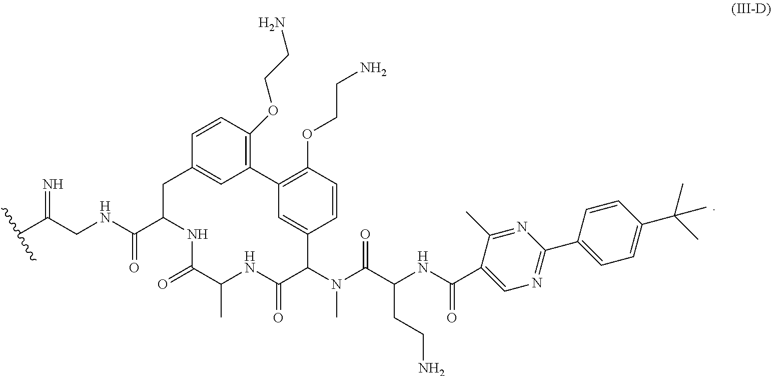 [SMILES: *C(=N)CNC(=O)C1Cc2ccc(OCCN)c(c2)-c2cc(ccc2OCCN)C(N(C)C(=O)C(CCN)NC(=O)c2cnc(-c3ccc(C(C)(C)C)cc3)nc2C)C(=O)NC(C)C(=O)N1]

110. The inhibited peptide of any one of claims 92-109, wherein the bond is a covalent bond.

111. A method of inhibiting a peptide, comprising contacting a peptide comprising a serine-lysine catalytic dyad or a serine-serine lysine catalytic triad with a compound of structural Formula (IV): 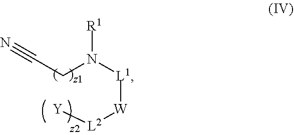
or a pharmaceutically acceptable salt thereof,
wherein:
n1 is an integer from 0 to 4;
m1 and v1 are independently 1 or 2;
z1 is an integer from 1 to 4;
z2 is 0 or 1;
R¹ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W is a substituted or unsubstituted linear peptide or a substituted or unsubstituted cyclic peptide, wherein the peptide comprises at least three of: natural amino acid residues, unnatural amino acid residues, or a combination thereof;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —O—, —NR⁹—, —S—, —C(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —SOₙ₁R^{1A}, —SOᵥ₁NR^{1B}R^{1C}, —NHNR^{1B}R^{1C}, —ONR^{1B}R^{1C}, —NHC(O)NHNR^{1B}R^{1C}, —NHC(O)NR^{1B}R^{1C}, —NR^{1B}R^{1C}, —C(O)R^{1D}, —C(O)OR^{1D}, —C(O)NR^{1B}R^{1C}, —OR^{1A}, —NR^{1B}SO₂R^{1A}, —NR^{1B}C(O)R^{1D}, —NR^{1B}C(O)OR^{D}, —NR^{B}OR^{D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a hydrophobic tail, or a bacterial protein or a fragment or homolog thereof; and
R^{1A}, R^{1B}, R^{1C}, and R^{1D} are independently hydrogen, halogen, —CF₃, —CCl₃, —CBr₃, —Cl₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl,
wherein the compound of Formula (IV) forms a bond to an amino group of the lysine in the dyad or triad.

112. The method of claim 111, wherein the compound has structural Formula (IV-C): 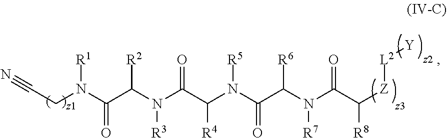
or a pharmaceutically acceptable salt thereof,
wherein:
z3 is an integer from 0 to 20;
R², R⁴, and R⁶ are independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R² and R⁶; or R² and R⁸ may optionally be joined to form a substituted or unsubstituted heterocycloalkyl;
R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl- C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl- C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H) [optionally substituted(C₂-C₆)alkyl]-OR^{8A}, —(C₁-C₆)alkylN(H)C(O)(C₁-C₆)alkyl-OR^{A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl;
Z is independently a substituted or unsubstituted natural amino acid or a substituted or unsubstituted unnatural amino acid, wherein R² and a Z amino acid; R⁴ and a Z amino acid; R⁶ and a Z amino acid; or two Z amino acids may optionally be joined by a linking moiety, L³, to form a substituted or unsubstituted heterocycloalkyl;
L³ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R^{8A} is independently hydrogen or —(C₁-C₆)alkyl;
R^{8B} and R^{8C} are independently hydrogen or optionally substituted —(C₁-C₆)alkyl or R^{8B} and R^{8C} and the nitrogen atom to which they are attached optionally form a heterocycloalkyl ring;
R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R¹⁴)₂, —C(O)N(R¹⁴)₂, or —SO₂N(R¹⁴)₂; or R^{8D} and R^{8E} and the nitrogen atom to which they are attached optionally form a heterocycloalkyl ring; and
R¹⁴ is independently hydrogen or —(C₁-C₆)alkyl; or two R¹⁴ groups and the nitrogen atom to which they are attached optionally form a heterocycloalkyl ring.

113. The method of claim 112, wherein R² and R⁶ are joined to form a substituted or unsubstituted heterocycloalkyl.

114. The method of claim 112 or 113, wherein z3 is 0.

115. The method of any one of claims 112-114, wherein the compound has structural Formula (V): 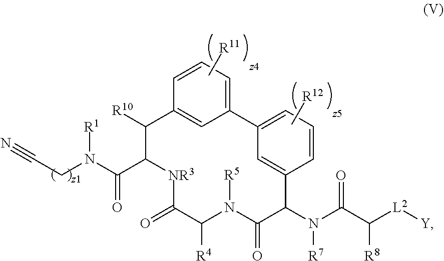
or a pharmaceutically acceptable salt thereof,
wherein:
z4 and z5 are independently an integer from 0 to 4;
R⁴ and R¹⁰ are independently hydrogen,
halogen, —CCl₃, —CBr₃, —CF₃, —Cl₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein R² and R⁶; or R² and R⁸ may optionally be joined to form a substituted or unsubstituted heterocycloalkyl;
R³, R⁵, and R⁷ are independently hydrogen,
halogen, —CCl₃, —CBr₃, —CF₃, —CI₃, —OH, —NH₂, —COOH, —CONH₂, —SH, —SO₃H, —SO₄H, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, —NH₂, —(C₁-C₆)alkyl, —(C₁-C₆)alkyl-OR^{8A}, —(C₁-C₆)alkyl-SR^{8A}, —(C₁-C₆)alkyl- C(O)OR^{8A}, —(C₁-C₆)alkyl-NR^{8D}R^{8E}, —(C₁-C₆)alkyl-NR^{8A}OR^{8A}, —(C₁-C₆)alkyl-NHC(O)NR^{8A}OR^{8A}, —(C₁-C₆)alkyl-O—(C₁-C₆)alkyl-NR^{8B}R^{8C}, —(C₁-C₆)alkyl-CN, —(C₁-C₆)alkyl-NR^{8A}C(O)R^{8A}, —(C₁-C₆)alkyl- C(O)NR^{8B}R^{8C}, —(C₁-C₆)heteroalkyl-CO₂H, —(C₁-C₆)alkyl-S(O)(C₁-C₆)alkyl, —(C₁-C₆)alkyl-N(H)CH═NH, —(C₁-C₆)alkyl-C(NH₂)═NH, —(C₁-C₆)alkyl-N(H)C(═NH)NH₂, —(C₁-C₆)alkyl-N(H)S(O)₂NR^{8B}R^{8C}, —(C₁-C₆)alkyl-N(H)S(O)₂(C₁-C₆)alkyl, —(C₁-C₆)alkyl-N(H)—C(O)NR^{8B}R^{8C}, —(C₁-C₆)alkylC(O)N(H)[optionally substituted(C₂-C₆)alkyl]-OR^{8A}, —(C₁-C₆)alkylN(H)C(O)(C₁-C₆)alkyl-OR^{8A}, —(C₁-C₆)alkylC(O)N(H)heterocycloalkyl, —(C₁-C₆)alkylC(O)NR^{8B}R^{8C}, —(C₁-C₆)alkyl-N(H)—C(O)—(C₁-C₆)alkyl-NR^{8B}R^{8C}, —(C₁-C₆)alkyl-N(H)—(C₁-C₆)alkylC(O)NR^{8B}R^{8C}, —(C₁-C₆)alkyl-heterocycloalkyl, optionally substituted —(C₁-C₆)alkyl-N(H)heterocycloalkyl, or —(C₁-C₆)alkyl-heteroaryl;
R¹¹ is hydrogen,
halogen, —CCl₃, —CBr₃, —CF₃, —Cl₃, —CN, —OR^{2A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen,
halogen, —CCl₃, —CBr₃, —CF₃, —Cl₃, —CN, —OR^{3A}, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A} and R^{3A} are independently hydrogen, halogen, —CF₃, —CCl₃, —CBr₃, —CI₃, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1B} and R^{1C} substituents bonded to the same nitrogen atom may optionally be joined to form a substituted or unsubstituted heterocycloalkyl or substituted or unsubstituted heteroaryl;
R^{8A} is independently hydrogen or —(C₁-C₆)alkyl;
R^{8B} and R^{8C} are independently hydrogen or optionally substituted —(C₁-C₆)alkyl or R^{8B} and R^{8C} and the nitrogen atom to which they are attached optionally form a heterocycloalkyl ring;
R^{8D} and R^{8E} are independently hydrogen, —(C₁-C₆)alkyl, —(C₁-C₆)heteroalkyl, —(C₁-C₆)alkyl-CO₂H, —C(O)(C₁-C₆)alkyl, —C(O)O(C₁-C₆)alkyl, —C(O)O(C₁-C₆)haloalkyl, —C(═NH)(C₁-C₆)alkyl, —C(═NH)N(R¹⁴)₂, —C(O)N(R¹⁴)₂, or —SO₂N(R¹⁴)₂; or R^{8D} and R^{8E} and the nitrogen atom to which they are attached optionally form a heterocycloalkyl ring; and
R¹⁴ is independently hydrogen or —(C₁-C₆)alkyl; or two R¹⁴ groups and the nitrogen atom to which they are attached optionally form a heterocycloalkyl ring.

116. The method of claim 115, wherein the compound has structural Formula (V-A): 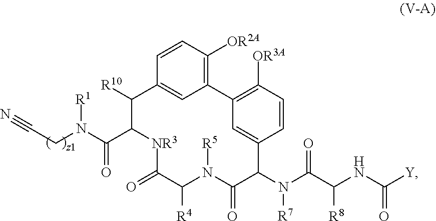
or a pharmaceutically acceptable salt thereof,
wherein R^{2A} and R^{3A} are independently substituted or unsubstituted alkyl or substituted or unsubstituted heteroalkyl.

117. The method of claim 116, wherein Y is hydrogen, substituted or unsubstituted aryl, or substituted or unsubstituted heteroaryl.

118. The method of claim 117, wherein R^{2A} and R^{3A} are independently substituted or unsubstituted heteroalkyl.

119. The method of claim 118, wherein the compound has structural Formula (V-B): 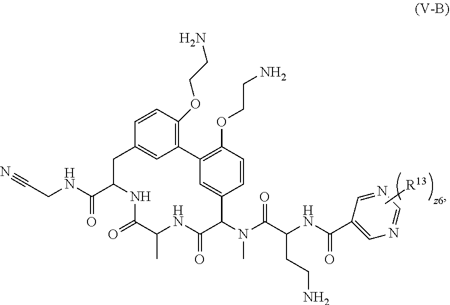
or a pharmaceutically acceptable salt thereof,
wherein:
z6 is an integer from 0 to 3; and
R¹³ is independently hydrogen,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

120. The method of claim 119, wherein R¹³ is independently substituted or unsubstituted alkyl, substituted or unsubstituted aryl, or substituted or unsubstituted heteroaryl.

121. The method of claim 120, wherein the compound has structural Formula (V-C): 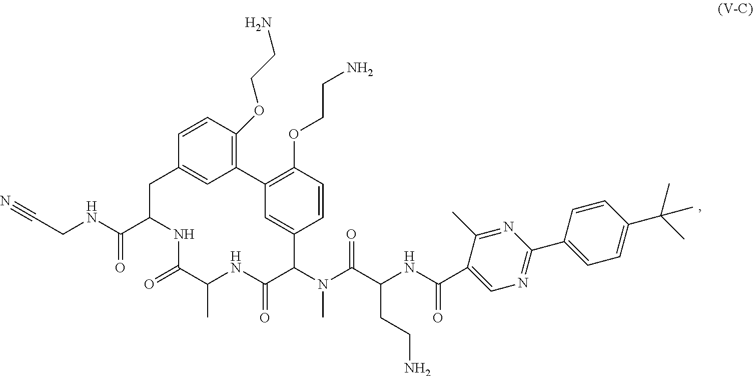 [SMILES: Cc1nc(-c2ccc(C(C)(C)C)cc2)ncc1C(=O)NC(CCN)C(=O)N(C)C1C(=O)NC(C)C(=O)NC(C(=O)NCC#N)Cc2ccc(OCCN)c(c2)-c2cc1ccc2OCCN]
or a pharmaceutically acceptable salt thereof.


SEQUENCE LISTING (6 sequences)

SEQ ID NO: 1 - protein, 10 aa, Artificial Sequence
  Description of Artificial Sequence Synthetic peptide
  MOD_RES (3)..(3): Any amino acid
  MOD_RES (6)..(6): Any amino acid
  MOD_RES (10)..(10): Any amino acid
PSXSMXPTLX

SEQ ID NO: 2 - protein, 9 aa, Artificial Sequence
  Description of Artificial Sequence Synthetic peptide
  MOD_RES (2)..(2): Any amino acid
  MOD_RES (4)..(4): Any amino acid
  MOD_RES (6)..(6): Any amino acid
  MOD_RES (8)..(9): Any amino acid
DXIXVXKXX

SEQ ID NO: 3 - protein, 10 aa, Artificial Sequence
  Description of Artificial Sequence Synthetic peptide
  MOD_RES (4)..(5): Any amino acid
  MOD_RES (8)..(9): Any amino acid
RGDXXVFXXP

SEQ ID NO: 4 - protein, 11 aa, Artificial Sequence
  Description of Artificial Sequence Synthetic peptide
  MOD_RES (1)..(1): Tyr or Phe
  MOD_RES (2)..(2): Ile or Val
  MOD_RES (5)..(6): Any amino acid
  MOD_RES (8)..(9): Any amino acid
XXKRXXGXXGD

SEQ ID NO: 5 - protein, 6 aa, Artificial Sequence
  Description of Artificial Sequence Synthetic peptide
LDYIKR

SEQ ID NO: 6 - protein, 7 aa, Artificial Sequence
  Description of Artificial Sequence Synthetic peptide
  MOD_RES (1)..(1): DAP
XPAKAAE